SEQUENCE LISTING (149 sequences)

SEQ ID NO: 1 - protein, 446 aa, synthetic construct
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 116 aa, synthetic construct
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 3 - protein, 5 aa, synthetic construct
DYGMH

SEQ ID NO: 4 - protein, 17 aa, synthetic construct
YISSGSSTIYYADTVKG

SEQ ID NO: 5 - protein, 7 aa, synthetic construct
RGLLLDY

SEQ ID NO: 6 - protein, 19 aa, synthetic construct
MGWSCIILFLVATATGVHS

SEQ ID NO: 7 - protein, 218 aa, synthetic construct
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 111 aa, synthetic construct
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIK

SEQ ID NO: 9 - protein, 15 aa, synthetic construct
RASKSVSTSSYSYMH

SEQ ID NO: 10 - protein, 7 aa, synthetic construct
YASYLES

SEQ ID NO: 11 - protein, 9 aa, synthetic construct
QHSREFPWT

SEQ ID NO: 12 - protein, 19 aa, synthetic construct
MGWSCIILFLVATATGVHS

SEQ ID NO: 13 - protein, 446 aa, synthetic construct
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 116 aa, synthetic construct
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 15 - protein, 5 aa, synthetic construct
NYGMH

SEQ ID NO: 16 - protein, 5 aa, synthetic construct
BYGMH

SEQ ID NO: 17 - protein, 17 aa, synthetic construct
YISSGSSTIYYADTVKG

SEQ ID NO: 18 - protein, 7 aa, synthetic construct
RGLLLDY

SEQ ID NO: 19 - protein, 19 aa, synthetic construct
MGWSCIILFLVATATGVHS

SEQ ID NO: 20 - protein, 218 aa, synthetic construct; identical to SEQ ID NO: 7

SEQ ID NO: 21 - protein, 111 aa, synthetic construct; identical to SEQ ID NO: 8

SEQ ID NO: 22 - protein, 15 aa, synthetic construct
RASKSVSTSSYSYMH

SEQ ID NO: 23 - protein, 7 aa, synthetic construct
YASYLES

SEQ ID NO: 24 - protein, 9 aa, synthetic construct
QHSREFPWT

SEQ ID NO: 25 - protein, 19 aa, synthetic construct
MGWSCIILFLVATATGVHS

SEQ ID NO: 26 - protein, 17 aa, synthetic construct
YISSGSSTIYYADSVKG

SEQ ID NO: 27 - protein, 17 aa, synthetic construct
YISSSSSTIYYADSVKG

SEQ ID NO: 28 - protein, 15 aa, synthetic construct
RASKSVSTSSYSYLA

SEQ ID NO: 29 - protein, 15 aa, synthetic construct
RASKTVSTSSYSYMH

SEQ ID NO: 30 - protein, 7 aa, synthetic construct
YASYLQS

SEQ ID NO: 31 - protein, 17 aa, synthetic construct
  VARIANT 5: note = X is separately any amino acid
  VARIANT 14: note = X is separately any amino acid
YISSXSSTIYYADXVKG

SEQ ID NO: 32 - protein, 15 aa, synthetic construct
  VARIANT 5: note = X is separately any amino acid
  VARIANT 14..15: note = X are separately any amino acids
RASKXVSTSSYSYXX

SEQ ID NO: 33 - protein, 7 aa, synthetic construct
  VARIANT 6: note = X is any amino acid
YASYLXS

SEQ ID NO: 34 - protein, 5 aa, synthetic construct
QYGMH

SEQ ID NO: 35 - protein, 5 aa, synthetic construct
EYGMH

SEQ ID NO: 36 - skipped

SEQ ID NO: 37 - protein, 5 aa, synthetic construct
  VARIANT 5: note = X is K or R
QYGMX

SEQ ID NO: 38 - protein, 5 aa, synthetic construct
  VARIANT 5: note = X is K or R
EYGMX

SEQ ID NO: 39 - protein, 17 aa, synthetic construct
YISSGSSTIYYAETVKG

SEQ ID NO: 40 - protein, 17 aa, synthetic construct
  VARIANT 16: note = X is R or H
YISSGSSTIYYADTVXG

SEQ ID NO: 41 - protein, 17 aa, synthetic construct
  VARIANT 16: note = X is R or H
YISSGSSTIYYAETVXG

SEQ ID NO: 42 - protein, 7 aa, synthetic construct
  VARIANT 1: note = X is K or H
XGLLLDY

SEQ ID NO: 43 - protein, 7 aa, synthetic construct
RGLLLEY

SEQ ID NO: 44 - protein, 7 aa, synthetic construct
  VARIANT 1: note = X is K or H
XGLLLEY

SEQ ID NO: 45 - protein, 15 aa, synthetic construct
  VARIANT 1: note = X is K or H
XASKSVSTSSYSYMH

SEQ ID NO: 46 - protein, 15 aa, synthetic construct
  VARIANT 4: note = X is R or H
RASXSVSTSSYSYMH

SEQ ID NO: 47 - protein, 15 aa, synthetic construct
  VARIANT 15: note = X is K or R
RASKSVSTSSYSYMX

SEQ ID NO: 48 - protein, 15 aa, synthetic construct
  VARIANT 1: note = X is K or H
  VARIANT 4: note = X is R or H
XASXSVSTSSYSYMH

SEQ ID NO: 49 - protein, 15 aa, synthetic construct
  VARIANT 1: note = X is K or H
  VARIANT 15: note = X is K or R
XASKSVSTSSYSYMX

SEQ ID NO: 50 - protein, 15 aa, synthetic construct
  VARIANT 4: note = X is R or H
  VARIANT 15: note = X is K or R
RASXSVSTSSYSYMX

SEQ ID NO: 51 - protein, 7 aa, synthetic construct
YASYLDS

SEQ ID NO: 52 - protein, 9 aa, synthetic construct
  VARIANT 2: note = X is K or R
QXSREFPWT

SEQ ID NO: 53 - protein, 9 aa, synthetic construct
  VARIANT 4: note = X is K or H
QHSXEFPWT

SEQ ID NO: 54 - protein, 9 aa, synthetic construct
QHSRDFPWT

SEQ ID NO: 55 - protein, 9 aa, synthetic construct
  VARIANT 2: note = X is K or R
  VARIANT 4: note = X is K or H
QXSXEFPWT

SEQ ID NO: 56 - protein, 9 aa, synthetic construct
  VARIANT 2: note = X is K or R
QXSRDFPWT

SEQ ID NO: 57 - protein, 9 aa, synthetic construct
  VARIANT 4: note = X is K or H
QHSXDFPWT

SEQ ID NO: 58 - skipped

SEQ ID NO: 59 - protein, 17 aa, synthetic construct
  VARIANT 5: note = X is G or S
  VARIANT 13: note = X is D or E
  VARIANT 14: note = X is T or S
  VARIANT 16: note = X is K, R, or H
YISSXSSTIYYAXXVXG

SEQ ID NO: 60 - protein, 7 aa, synthetic construct
  VARIANT 1: note = X is K, R, or H
  VARIANT 6: note = X is D or E
XGLLLXY

SEQ ID NO: 61 - protein, 15 aa, synthetic construct
  VARIANT 1: note = X is K, R, or H
  VARIANT 4: note = X is K, R, or H
  VARIANT 5: note = X is T or S
  VARIANT 14: note = X is M or L
  VARIANT 15: note = X is K, R, H, or A
XASXXVSTSSYSYXX

SEQ ID NO: 62 - protein, 7 aa, synthetic construct
  VARIANT 6: note = X is D, E, N, or Q
YASYLXS

SEQ ID NO: 63 - protein, 9 aa, synthetic construct
  VARIANT 2: note = X is K, R, or H
  VARIANT 4: note = X is K, R, or H
  VARIANT 5: note = X is D or E
QXSXXFPWT

SEQ ID NO: 64 - protein, 116 aa, synthetic construct
EVQLVESGGGLVQPGGSLRLSCSASGFTFSNYGMHWVRQAPGKGLEYVSYISSGSSTIYYADTVKGRFTISRDNSKNTLYLQMSSLRAEDTAVYYCVKRGLLLDYWGQGTLVTVSS

SEQ ID NO: 65 - protein, 116 aa, synthetic construct
EVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 66 - protein, 116 aa, synthetic construct
EVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 67 - protein, 116 aa, synthetic construct
EVQLVQSGGGLIQPGGSLRLSCAASGFTFSNYGMHWVRQAPGKGLEWVSYISSGSSTIYYADSVKGRFTISRDNSKNTLYLQMNSLRAEDTAVYYCARRGLLLDYWGQGTTVTVSS

SEQ ID NO: 68 - protein, 116 aa, synthetic construct
EVQLVESGGGLIQPGGSLRLSCAASGFTFSNYGMHWVRQAPGKGLEWVSYISSGSSTIYYADSVKGRFTISRDNSKNTLYLQMTSLRAEDTAVYYCARRGLLLDYWGQGTTLTVSS

SEQ ID NO: 69 - protein, 116 aa, synthetic construct
EVQLQESGGGVVQPGGSLRLSCAASGFTFSNYGMHWIRQAPGKGLEWVSYISSGSSTIYYADSVKGRFTISRDNSKNTLYLQMNSLRSEDTAVYYCARRGLLLDYWGQGTLVTVSS

SEQ ID NO: 70 - protein, 116 aa, synthetic construct
EVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 71 - protein, 116 aa, synthetic construct
EVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 72 - protein, 116 aa, synthetic construct
EVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 73 - protein, 116 aa, synthetic construct
EVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 74 - protein, 111 aa, synthetic construct
DIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGQGTKVEIK

SEQ ID NO: 75 - protein, 111 aa, synthetic construct
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIK

SEQ ID NO: 76 - protein, 111 aa, synthetic construct
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIK

SEQ ID NO: 77 - protein, 111 aa, synthetic construct
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIK

SEQ ID NO: 78 - protein, 111 aa, synthetic construct
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIK

SEQ ID NO: 79 - protein, 111 aa, synthetic construct
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIK

SEQ ID NO: 80 - protein, 111 aa, synthetic construct
DIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIK

SEQ ID NO: 81 - protein, 111 aa, synthetic construct
DIQMTQSPSSLSASVGDRVTITCRASKSVSTSSYSYLAWYQQKPEKAPKLLIKYASYLQSGVPSRFSGSGSGTDFTLTISSLQPEDFATYYCQHSREFPWTFGAGTKLELK

SEQ ID NO: 82 - protein, 111 aa, synthetic construct
DIQMTQSPSSLSASVGDRVTISCRASKSVSTSSYSYMHWYQQKPEKAPKLLIKYASYLQSGVPSRFSGSGSGTDFTLTISSLQPEDVATYYCQHSREFPWTFGAGTKLELK

SEQ ID NO: 83 - protein, 515 aa, synthetic construct
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 84 - protein, 515 aa, synthetic construct
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 85 - protein, 515 aa, synthetic construct
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 86 - protein, 515 aa, synthetic construct
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 87 - protein, 515 aa, synthetic construct
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 88 - protein, 515 aa, synthetic construct
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 89 - protein, 515 aa, synthetic construct
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 90 - protein, 515 aa, synthetic construct
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 91 - protein, 515 aa, synthetic construct
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 92 - protein, 515 aa, synthetic construct
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 93 - protein, 515 aa, synthetic construct
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 94 - protein, 515 aa, synthetic construct
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 95 - protein, 515 aa, synthetic construct
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 96 - protein, 515 aa, synthetic construct
DIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 97 - protein, 515 aa, synthetic construct
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 98 - protein, 515 aa, synthetic construct
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 99 - protein, 116 aa, synthetic construct
EVQLVESGGGLVQPGGSLRLSCAASGFTFSNYGMHWVRQAPGKGLEWVSYISSGSSTIYYADTVKGRFTISRDNAKNSLYLQMNSLRAEDTAVYYCARRGLLLDYWGQGTLVTVSS

SEQ ID NO: 100 - protein, 116 aa, synthetic construct
EVQLVESGGGLVQPGGSLRLSCAASGFTFSNYGMHWVRQAPGKGLEWVAYISSGSSTIYYADTVKGRFTISRDNAKNSLYLQMNSLRAEDTAMYYCARRGLLLDYWGQGTLVTVSS

SEQ ID NO: 101 - protein, 116 aa, synthetic construct
EVQLVESGGGLVQPGGSLRLSCAASGFTFSNYGMHWVRQAPGKGLEWVAYISSGSSTIYYADTVKGRFTISRDNAKNSLYLQMTSLRAEDTAMYYCARRGLLLDYWGQGTLVTVSS

SEQ ID NO: 102 - protein, 116 aa, synthetic construct
EVQLVESGGGLVQPGGSLRLSCAASGFTFSNYGMHWVRQAPEKGLEWVAYISSGSSTIYYADTVKGRFTISRDNAKNSLYLQMTSLRAEDTAMYYCARRGLLLDYWGQGTLVTVSS

SEQ ID NO: 103 - protein, 116 aa, synthetic construct
EVQLVESGGGLVQPGGSLRLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSS

SEQ ID NO: 104 - protein, 116 aa, synthetic construct
EVQLVESGGGLVQPGGSRKLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSS

SEQ ID NO: 105 - protein, 116 aa, synthetic construct
EVQLVESGGGLVQPGGSRKLSCAASGFTFSNYGMHWVRQAPEKGLEWVAYISSGSSTIYYADTVKGRFTISRDNAKNTLFLQMTSLRAEDTAVYYCARRGLLLDYWGQGTLVTVSS

SEQ ID NO: 106 - protein, 446 aa, synthetic construct
EVQLVESGGGLVQPGGSLRLSCAASGFTFSNYGMHWVRQAPGKGLEWVSYISSGSSTIYYADTVKGRFTISRDNAKNSLYLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 446 aa, synthetic construct
EVQLVESGGGLVQPGGSLRLSCAASGFTFSNYGMHWVRQAPGKGLEWVAYISSGSSTIYYADTVKGRFTISRDNAKNSLYLQMNSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 446 aa, synthetic construct
EVQLVESGGGLVQPGGSLRLSCAASGFTFSNYGMHWVRQAPGKGLEWVAYISSGSSTIYYADTVKGRFTISRDNAKNSLYLQMTSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 446 aa, synthetic construct
EVQLVESGGGLVQPGGSLRLSCAASGFTFSNYGMHWVRQAPEKGLEWVAYISSGSSTIYYADTVKGRFTISRDNAKNSLYLQMTSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 446 aa, synthetic construct
EVQLVESGGGLVQPGGSLRLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 446 aa, synthetic construct
EVQLVESGGGLVQPGGSRKLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 446 aa, synthetic construct
EVQLVESGGGLVQPGGSRKLSCAASGFTFSNYGMHWVRQAPEKGLEWVAYISSGSSTIYYADTVKGRFTISRDNAKNTLFLQMTSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 465 aa, synthetic construct
MGWSCIILFLVATATGVHSEVQLVESGGGLVQPGGSLRLSCAASGFTFSNYGMHWVRQAPGKGLEWVSYISSGSSTIYYADTVKGRFTISRDNAKNSLYLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 465 aa, synthetic construct
MGWSCIILFLVATATGVHSEVQLVESGGGLVQPGGSLRLSCAASGFTFSNYGMHWVRQAPGKGLEWVAYISSGSSTIYYADTVKGRFTISRDNAKNSLYLQMNSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 465 aa, synthetic construct
MGWSCIILFLVATATGVHSEVQLVESGGGLVQPGGSLRLSCAASGFTFSNYGMHWVRQAPGKGLEWVAYISSGSSTIYYADTVKGRFTISRDNAKNSLYLQMTSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 465 aa, synthetic construct
MGWSCIILFLVATATGVHSEVQLVESGGGLVQPGGSLRLSCAASGFTFSNYGMHWVRQAPEKGLEWVAYISSGSSTIYYADTVKGRFTISRDNAKNSLYLQMTSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 465 aa, synthetic construct
MGWSCIILFLVATATGVHSEVQLVESGGGLVQPGGSLRLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 465 aa, synthetic construct
MGWSCIILFLVATATGVHSEVQLVESGGGLVQPGGSRKLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 465 aa, synthetic construct
MGWSCIILFLVATATGVHSEVQLVESGGGLVQPGGSRKLSCAASGFTFSNYGMHWVRQAPEKGLEWVAYISSGSSTIYYADTVKGRFTISRDNAKNTLFLQMTSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 111 aa, synthetic construct
DIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGGGTKVEIK

SEQ ID NO: 121 - protein, 111 aa, synthetic construct
DIVLTQSPASLAVSPGQRATITCRASKSVSTSSYSYMHWYQQKPGQPPKLLIKYASYLESGVPARFSGSGSGTDFTLTINPVEAEDTATYYCQHSREFPWTFGGGTKVEIK

SEQ ID NO: 122 - protein, 111 aa, synthetic construct
DIVLTQSPASLAVSPGQRATITCRASKSVSTSSYSYMHWYQQKPGQPPKLLIKYASYLESGVPARFSGSGSGTDFTLTINPVEAEDTANYYCQHSREFPWTFGGGTKVEIK

SEQ ID NO: 123 - protein, 111 aa, synthetic construct
DIVLTQSPASLAVSPGQRATITCRASKSVSTSSYSYMHWYQQKPGQPPKLLIKYASYLESGVPARFSGSGSGTDFTLTINPVEAEDAATYYCQHSREFPWTFGGGTKVEIK

SEQ ID NO: 124 - protein, 111 aa, synthetic construct
DIVLTQSPASLAVSPGQRATITCRASKSVSTSSYSYMHWYQQKPGQPPKLLIKYASYLESGVPARFSGSGSGTDFTLNIHPVEAEDTANYYCQHSREFPWTFGGGTKVEIK

SEQ ID NO: 125 - protein, 111 aa, synthetic construct
DIVLTQSPASLAVSPGQRATISCRASKSVSTSSYSYMHWYQQKPGQPPKLLIKYASYLESGVPARFSGSGSGTDFTLNIHPVEEEDTANYYCQHSREFPWTFGGGTKVEIK

SEQ ID NO: 126 - protein, 218 aa, synthetic construct
DIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 218 aa, synthetic construct
DIVLTQSPASLAVSPGQRATITCRASKSVSTSSYSYMHWYQQKPGQPPKLLIKYASYLESGVPARFSGSGSGTDFTLTINPVEAEDTATYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 218 aa, synthetic construct
DIVLTQSPASLAVSPGQRATITCRASKSVSTSSYSYMHWYQQKPGQPPKLLIKYASYLESGVPARFSGSGSGTDFTLTINPVEAEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 218 aa, synthetic construct
DIVLTQSPASLAVSPGQRATITCRASKSVSTSSYSYMHWYQQKPGQPPKLLIKYASYLESGVPARFSGSGSGTDFTLTINPVEAEDAATYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - protein, 218 aa, synthetic construct
DIVLTQSPASLAVSPGQRATITCRASKSVSTSSYSYMHWYQQKPGQPPKLLIKYASYLESGVPARFSGSGSGTDFTLNIHPVEAEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 218 aa, synthetic construct
DIVLTQSPASLAVSPGQRATISCRASKSVSTSSYSYMHWYQQKPGQPPKLLIKYASYLESGVPARFSGSGSGTDFTLNIHPVEEEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 237 aa, synthetic construct
MGWSCIILFLVATATGVHSDIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 237 aa, synthetic construct
MGWSCIILFLVATATGVHSDIVLTQSPASLAVSPGQRATITCRASKSVSTSSYSYMHWYQQKPGQPPKLLIKYASYLESGVPARFSGSGSGTDFTLTINPVEAEDTATYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 237 aa, synthetic construct
MGWSCIILFLVATATGVHSDIVLTQSPASLAVSPGQRATITCRASKSVSTSSYSYMHWYQQKPGQPPKLLIKYASYLESGVPARFSGSGSGTDFTLTINPVEAEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - protein, 237 aa, synthetic construct
MGWSCIILFLVATATGVHSDIVLTQSPASLAVSPGQRATITCRASKSVSTSSYSYMHWYQQKPGQPPKLLIKYASYLESGVPARFSGSGSGTDFTLTINPVEAEDAATYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 237 aa, synthetic construct
MGWSCIILFLVATATGVHSDIVLTQSPASLAVSPGQRATITCRASKSVSTSSYSYMHWYQQKPGQPPKLLIKYASYLESGVPARFSGSGSGTDFTLNIHPVEAEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 237 aa, synthetic construct
MGWSCIILFLVATATGVHSDIVLTQSPASLAVSPGQRATISCRASKSVSTSSYSYMHWYQQKPGQPPKLLIKYASYLESGVPARFSGSGSGTDFTLNIHPVEEEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - RNA, 720 nt, synthetic construct
  source 1..720: mol_type = other RNA
  mRNA 1..720: note = mRNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 139-142 - skipped (4 entries)

SEQ ID NO: 143 - protein, 31 aa, synthetic construct
RASKSVSTSSYSYMHWYQQKPGQPPKLLIKY

SEQ ID NO: 144 - protein, 31 aa, synthetic construct
RASKTVSTSSYSYMHWYQQKPGQPPKLLIKY

SEQ ID NO: 145 - protein, 33 aa, synthetic construct
RVTITCRASKSVSTSSYSYMHWYQQKPGKAPKL

SEQ ID NO: 146 - protein, 32 aa, synthetic construct
  VARIANT 1: note = X can be any residue, but preferentially a basic residue R or K
  VARIANT 30: note = X can be any residue, but preferentially a basic residue R or K
XRASKTVSTSSYSYMHWYQQKPGQPPKLLXKY

SEQ ID NO: 147 - skipped

SEQ ID NO: 148 - protein, 115 aa, synthetic construct
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVS

SEQ ID NO: 149 - protein, 132 aa, synthetic construct
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGS

(19) United States
(12) Patent Application Publication
(10) Pub. No.: US 2025/0161484 A1
(43) Pub. Date: May 22, 2025

(54) COMPOSITIONS AND METHODS FOR TREATING DISEASE

(71) Applicants: Yale University, New Haven, CT (US); Gennao Bio, Inc., Pennington, NJ (US)

(72) Inventors: Elias Quijano, Durham, CT (US); Peter Glazer, Guilford, CT (US); Stephen Squinto, New York, NY (US); Dale Ludwig, Rockaway, NJ (US); Bruce Turner, Bryn Mawr, PA (US)

(21) Appl. No.: 18/841,043

(22) PCT Filed: Mar. 3, 2023
(86) PCT No.: PCT/US2023/063677

§ 371 Date: Aug. 23, 2024

Related U.S. Application Data
(60) Provisional application No. 63/316,341, filed on Mar. 3, 2022.
(60) Provisional application No. 63/316,347, filed on Mar. 3, 2022.

(51) Int. Cl. A61K 48/00; A61K 38/17; A61P 1/16

(52) U.S. Cl. CPC A61K 48/0033; A61K 38/1709; A61K 48/005; A61P 1/16

(57) ABSTRACT

Compositions and methods are provided for treating diseases and disorders, e.g., cardiac muscle, hepatic, lung, and brain, by administering a complex formed between a therapeutic mRNA polynucleotide and a 3E10 antibody or antigen binding fragment thereof. In some instances, the complexes are stabilized through a molar ratio of 3E10 antibody or antigen binding fragment thereof to therapeutic polynucleotide of at least about 2:1.

### CROSS REFERENCE TO RELATED APPLICATIONS

This application claims priority to U.S. Provisional Application No. 63/316,341 filed Mar. 3, 2022, and U.S. Provisional Application No. 63/316,347, filed Mar. 3, 2022, the disclosure of which is herein incorporated by reference in its entirety.

### TECHNICAL FIELD

The present disclosure provides compositions and methods for delivering therapeutic mRNA molecules, in vivo, via 3E10 antibodies and antigen binding fragments thereof.

### BACKGROUND

Protein replacement therapy has been used as conventional therapy for various genetic disorders that cause protein deficiencies, such as blood clotting disorders (e.g., hemophilia A, hemophilia B, von Willebrand's disease, and Factor VII deficiency) and lysosomal storage diseases (e.g., Fabry's disease, Gaucher disease, and Pompe disease). Next generation gene therapies are currently being developed for many of these diseases, as well as other genetic disorders such as cystic fibrosis, for which enzyme replacement therapy is not a viable option. However, therapeutic mRNA delivery is an attractive option for treating such diseases because it does not result in the integration of an exogenous nucleic acid sequence into the subject's genome and eliminates the need to transcribe an exogenous copy of the gene.

Therapeutic mRNA delivery potentially avoids many of the limitations and risks associated with viral vector and synthetic liposome-based gene therapy, including complexity of production, limited packaging capacity, and unfavorable immunological features, which restrict gene therapy applications and hold back the potential for preventive gene therapy (Seow and Wood, Mol Ther. 17 (5): 767-777 (2009).

However, mRNA therapy is limited by the need for improved delivery systems. For instance, mRNA does not readily cross the cell membrane. Conventional approaches to overcoming this obstacle include packaging mRNA in liposomal-based delivery vehicles, which present similar immunological challenges as DNA-based therapies. Further, mRNA is readily degraded by extracellular ribonucleases present in skin, tissues, and blood. Kowalski P S et al., Mol Ther., 27 (4): 710-28 (2019), the content of which is incorporated by reference herein.

### SUMMARY

Given the background above, improved methods for treating cardiac muscle, hepatic, lung, and brain diseases and disorders are needed. mRNA therapies present a promising path for treatment of these diseases because the underlying genetics of disease etiology are well characterized. Advantageously, the present disclosure provides compositions and methods for mRNA therapy of cardiac, liver, lung, and brain diseases are needed that are not reliant upon liposomal or viral vector based nucleic acid delivery. In some aspects, these compositions and methods are based on, at least in part, on the discovery that 3E10 antibodies or antigen-binding fragments thereof can be used to efficiently deliver therapeutic mRNA molecules to cardiac, hepatic, lung, and neuronal tissue in vivo.

In some embodiments, the advantageous properties of the compositions and methods described herein are based, at least in part, on the discovery that use of higher molar ratios of 3E10 antibody or antigen binding fragment thereof to mRNA molecule result in greater protection of the mRNA molecule from RNA degradation. For instance, as described in Example 3 and illustrated in FIGS. 9A and 9B, while parental 3E10 and 3E10 (D31N) variant antibodies protected mRNA from RNAse A-mediated RNA degradation at molar ratios of 2:1 and 20:1, the protection afforded by the 20:1 molar ratio exceeded the protection afforded at 2:1. Advantageously, the increased mRNA protection afforded mRNA at higher 3E10 antibody or antigen binding fragment thereof concentrations is exploited in the compositions and methods described herein to improve the pharmacokinetic properties of therapeutic compositions delivering mRNA in vivo.

Accordingly, one aspect of the present disclosure provides methods for treating a cardiac muscle, hepatic, lung, or brain disease or disorder in a subject in need thereof, by parenterally administering a therapeutically effective amount of a composition comprising a complex formed between a therapeutic mRNA polynucleotide, and a 3E10 antibody or antigen binding fragment thereof.

Accordingly, another aspect of the present disclosure provides methods for treating a cardiac muscle, hepatic, lung, or brain disease or disorder in a subject in need thereof, by parenterally administering a therapeutically effective amount of a composition comprising a non-covalent complex formed between a therapeutic mRNA polynucleotide, and a 3E10 antibody or antigen binding fragment thereof.

Accordingly, another aspect of the present disclosure provides methods for treating a cardiac muscle, hepatic, lung, or brain disease or disorder in a subject in need thereof, by parenterally administering a therapeutically effective amount of a composition comprising a covalent complex formed between a therapeutic mRNA polynucleotide, and a 3E10 antibody or antigen binding fragment thereof.

In another aspect, the present disclosure provides pharmaceutical compositions of a complex formed between a therapeutic mRNA polynucleotide encoding a cardiac muscle, hepatic, lung, or neuronal polypeptide, and a 3E10 antibody or antigen binding fragment thereof, where the pharmaceutical composition has a molar ratio of 3E10 antibody or antigen binding fragment thereof to therapeutic polynucleotide of at least 2:1.

In some embodiments of the methods and compositions described herein, the 3E10 antibody or antigen binding fragment thereof includes a light chain variable region (VL) complementarity determining region (CDR) 1 comprising the amino acid sequence of 3E10-VL-CDR1 (SEQ ID NO: 9), a VL CDR2 comprising the amino acid sequence of 3E10-VL-CDR2 (SEQ ID NO: 10), a VL CDR3 comprising the amino acid sequence of 3E10-VL-CDR3 (SEQ ID NO: 11), a heavy chain variable region (VH) CDR1 comprising the amino acid sequence of 3E10-VH-CDR1a (SEQ ID NO: 16), a VH CDR2 comprising the amino acid sequence of 3E10-VH-CDR2 (SEQ ID NO: 4), and a VH CDR3 comprising the amino acid sequence of 3E10-VH-CDR3 (SEQ ID NO: 5).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates amino acid sequences for the parent 3E10 monoclonal antibody.

FIGS. 2A, 2B, and 2C illustrate amino acid sequences for the D3IN variant (FIG. 2A), other CDR variants (FIG. 2B), and additionally contemplated CDR variants (FIG. 2C) of the 3E10 monoclonal antibody, in accordance with some embodiments of the present disclosure.

FIG. 3 illustrates example charge-conserved CDR variants of the 3E10 monoclonal antibody, in accordance with various embodiments of the present disclosure.

FIG. 4 illustrates example CDR variants containing a combination of amino acid substitutions, charged-conserved amino acid substitutions, and rationally-designed amino acid substitutions of the 3E10 monoclonal antibody, in accordance with various embodiments of the present disclosure.

FIG. 5 illustrates a sequence alignment of examples of humanized 3E10 heavy chain variable regions, with CDRs underlined as indicated.

FIG. 6 illustrates a sequence alignment of examples of humanized 3E10 light chain variable regions, with CDRs and putative nuclear localization signals (NLS) underlined as indicated.

FIGS. 7A, 7B, 7C, 7D, and 7E collectively illustrate a sequence alignment of example of humanized di-scFv constructs of the 3E10 monoclonal antibody.

FIGS. 8A and 8B illustrate electrostatic surface potential renderings of a molecular model of a 3E10-scFv construct, revealing a putative Nucleic Acid Binding pocket (NAB1). FIG. 8A additionally shows predicted structural and electrostatic potential changes induced by amino acid substitutions at residue HC CDR1 residue 31. FIG. 8B is an illustration of molecular modeling of 3E10-scFv (Pymol) with NAB1 amino acid residues highlighted by punctate dots.

FIG. 8C illustrates mapping of the putative nucleic acid binding pocket, as identified by the molecular modeling shown in FIGS. 8A and 8B, onto the amino acid sequence of the 3E10-scFv construct.

FIGS. 9A and 9B show gel electrophoresis analysis mRNA protection assays performed with 3E10 (D31N)-mRNA constructs prepared at 20:1 (FIG. 9A) and 2:1 (FIG. 9B) molar ratios.

FIG. 10 is a bar graph quantifying the fluorescence in IVIS images of dose-dependent biodistribution of 3E10-D31N to tissues 24 hours following 100 μg or 200 μg intravenous injection of 3E10-D31N labeled with VivoTag680 into mice (Perkin Elmer), as described in Example 4.

FIG. 11 shows a histogram of cytosolic, membrane, nuclear protein, and gDNA fractions after administration of ⁸⁹Zr labeled isotype control, 3E10-WT, and 3E10-D31N antibodies, as described in Example 5.

FIG. 12 shows gel electrophoresis analysis of mRNA protection assays performed with complexes formed between 3E10 and a 14 kb mRNA encoding the human dystrophin protein, prepared at 1:1, 2:1, 5:1, 10:1 and 100:1 (3E10: mRNA) molar ratios, as described in Example 6.

FIG. 13 illustrates amino acid sequences of humanized 3E10 variable heavy (3E10-VH) domains, in accordance with various embodiments of the present disclosure.

FIG. 14 illustrates amino acid sequences of mature humanized 3E10 heavy chains (3E10-HC), lacking a signal peptide, in accordance with various embodiments of the present disclosure.

FIG. 15 illustrates amino acid sequences of humanized 3E10 heavy chains (3E10-HC), in accordance with various embodiments of the present disclosure.

FIG. 16 illustrates amino acid sequences of humanized 3E10 variable light (3E10-VL) domains, in accordance with various embodiments of the present disclosure.

FIG. 17 illustrates amino acid sequences of mature humanized 3E10 light chains (3E10-LC), lacking a signal peptide, in accordance with various embodiments of the present disclosure.

FIG. 18 illustrates amino acid sequences of humanized 3E10 light chains (3E10-LC), in accordance with various embodiments of the present disclosure.

FIG. 19 illustrates carrier DNA enhancing the delivery of fluorescently labeled mRNA complexed to 3E10-D31N in murine fetal tissue.

FIGS. 20A-20C illustrate delivery of fluorescently labeled oligos to skeletal muscle in MDX mice in a 2-dose regimen (FIG. 20A) and 3-dose regimen (FIG. 20B). FIG. 20C illustrates IVIS imaging of skeletal muscle tissue.

FIG. 21A-21C illustrate repeat dosing of fluorescently labeled 3E10-D31N (V66) in tumor bearing mice.

### DETAILED DESCRIPTION

The present disclosure provides compositions and methods for delivering therapeutic mRNA molecules, in vivo, that are not reliant upon the conventional viral-based or liposomal-based delivery methodologies associated with difficult and costly production, limited packaging capacity, and adverse immunological events. In some aspects, described in greater detail below, these compositions and methods are based on, at least in part, on the discovery that 3E10 antibodies or antigen-binding fragments thereof can be used to deliver therapeutic mRNA molecules efficiently to cardiac, hepatic, lung, and neuronal tissue in vivo.

Specifically, it was discovered that 3E10 antibodies or variants thereof, or antigen-binding fragments thereof help transport mRNA across the plasma membrane, into the cell cytoplasm. Thus, compositions and methods for using 3E10 antibodies or variants thereof, or antigen-binding fragments thereof to enhance delivery of mRNA, particularly to cardiac, hepatic, lung, and neuronal tissue, are provided.

### Definitions

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. Unless the context requires otherwise, it will be further understood that the terms “includes,” “comprising,” or any variation thereof, when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “including,” “includes,” “having,” “has,” “with,” or variants thereof are used in either the detailed description and/or the claims, such terms are intended to be inclusive in a manner similar to the term “comprising.”

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%.

By “antigen binding domain” or “ABD” herein is meant a set of six Complementary Determining Regions (CDRs) that, when present as part of a polypeptide sequence or sequences, specifically binds a target antigen as discussed herein. Thus, a “antigen binding domain” binds a nucleic acid antigen as outlined herein. As is known in the art, these CDRs are generally present as a first set of variable heavy CDRs (vhCDRs or VHCDRs) and a second set of variable light CDRs (vlCDRs or VLCDRs), each comprising three CDRs: vhCDR1, vhCDR2, vhCDR3 for the heavy chain and vlCDR1, vlCDR2 and vlCDR3 for the light. The CDRs are present in the variable heavy and variable light domains, respectively, and together form an Fv region. (See. Table 1 and related discussion above for CDR numbering schemes). Thus, in some cases, the six CDRs of the antigen binding domain are contributed by a variable heavy and a variable light domain. In a “Fab” format, the set of 6 CDRs are contributed by two different polypeptide sequences, the variable heavy domain (vh or VH; containing the vhCDR1, vhCDR2 and vhCDR3) and the variable light domain (vl or VL; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). In a scFv format, the vh and vl domains are covalently attached, generally through the use of a linker (a “scFv linker”) as outlined herein, into a single polypeptide sequence, which can be either (starting from the N-terminus) vh-linker-vl or vl-linker-vh, with the former being generally preferred (including optional domain linkers on each side, depending on the format used. In general, the C-terminus of the scFv domain is attached to the N-terminus of the hinge in the second monomer.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g. vhCDR1, vhCDR2 and vhCDR3) and the disclosure of each variable light region is a disclosure of the vlCDRs (e.g. vlCDR1, vlCDR2 and vlCDR3). A useful comparison of CDR numbering is as below, see Lafranc et al., Dev. Comp. Immunol. 27 (1): 55-77 (2003):

TABLE 1
CDR numbering schemes.

| | Kabat + | | | | | | |
|---|---|---|---|---|---|---|---|
| | Chothia | IMGT | Kabat | AbM | Chothia | Contact | Xencor |
| vhCDR1 | 26-35 | 27-38 | 31-35 | 26-35 | 26-32 | 30-35 | 27-35 |
| vhCDR2 | 50-65 | 56-65 | 50-65 | 50-58 | 52-56 | 47-58 | 54-61 |
| vhCDR3 | 95-102 | 105-117 | 95-102 | 95-102 | 95-102 | 93-101 | 103-116 |
| vlCDR1 | 24-34 | 27-38 | 24-34 | 24-34 | 24-34 | 30-36 | 27-38 |
| vlCDR2 | 50-56 | 56-65 | 50-56 | 50-56 | 50-56 | 46-55 | 56-62 |
| vlCDR3 | 89-97 | 105-117 | 89-97 | 89-97 | 89-97 | 89-96 | 97-105 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g, Kabat et al., supra (1991)). The EU index or EU index as in Kabat or EU numbering scheme refers to the numbering of the EU antibody. Kabat et al. collected numerous primary sequences of the variable regions of heavy chains and light chains. Based on the degree of conservation of the sequences, they classified individual primary sequences into the CDR and the framework and made a list thereof. See, SEQUENCES OF IMMUNOLOGICAL INTEREST, 5th edition, NIH publication, No. 91-3242, E. A. Kabat et al.; Edelman et al., 1969, Proc Natl Acad Sci USA 63:78-85, the contents of which are incorporated herein by reference. The modification can be an addition, deletion, or substitution.

By “target antigen” as used herein is meant the molecule that is bound specifically by the antigen binding domain comprising the variable regions of a given antibody. As discussed below, in the present case the target antigens are nucleic acids.

As described below, in some embodiments a parent polypeptide, for example an Fc parent polypeptide, is a human wild type sequence, such as the heavy constant domain or Fc region from IgG1, IgG2, IgG3 or IgG4, although human sequences with variants can also serve as “parent polypeptides”, for example the IgG1/2 hybrid of US Publication 2006/0134105 can be included. The protein variant sequence herein will preferably possess at least about 75% identity with a parent protein sequence, or at least about 80% identity with a parent protein sequence, and most preferably at least about 90% identity, more preferably at least about 95%, or at least about 98%, or at least about 99% sequence identity. In some embodiments, the protein variant sequence herein has at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity with a parent protein sequence. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain as compared to an Fc domain of human IgG1, IgG2, IgG3, or IgG4.

By “isotype” as used herein is meant any of the subclasses of immunoglobulins defined by the chemical and antigenic characteristics of their constant regions. It should be understood that therapeutic antibodies can also comprise hybrids of isotypes and/or subclasses.

By “Fab” or “Fab region” as used herein is meant a polypeptide that comprises the VH, CH1, VL, and CL immunoglobulin domains, generally on two different polypeptide chains (e.g. VH—CH1 on one chain and VL-CL on the other). Fab may refer to this region in isolation, or this region in the context of an antibody of the disclosure. In the context of a Fab, the Fab comprises an Fv region in addition to the CH1 and CL domains.

By “Fv” or “Fv fragment” or “Fv region” as used herein is meant a polypeptide that comprises the VL and VH domains of an ABD. Fv regions can be formatted as both Fabs (as discussed above, generally two different polypeptides that also include the constant regions as outlined above) and scFvs, where the vl and vh domains are combined (generally with a linker as discussed herein) to form an scFv.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (vh-linker-vl or vl-linker-vh). In the sequences depicted in the sequence listing and in the figures, the order of the vh and vl domain is indicated in the name, e.g. H.X_L. Y means N- to C-terminal is vh-linker-vl, and L. Y_H.X is vl-linker-vh.

By “Fc” or “Fc region” or “Fc domain” as used herein is meant the polypeptide comprising the CH2-CH3 domains of an IgG molecule, and in some cases, inclusive of the hinge. In EU numbering for human IgG1, the CH2-CH3 domain comprises amino acids 231 to 447, and the hinge is 216 to 230. Thus the definition of “Fc domain” includes both amino acids 231-447 (CH2-CH3) or 216-447 (hinge-CH2-CH3), or fragments thereof. An “Fc fragment” in this context may contain fewer amino acids from either or both of the N- and C-termini but still retains the ability to form a dimer with another Fc domain or Fc fragment as can be detected using standard methods, generally based on size (e.g. non-denaturing chromatography, size exclusion chromatography, etc.) Human IgG Fc domains are of particular use in the present disclosure, and can be the Fc domain from human IgG1, IgG2 or IgG4.

A “variant Fc domain” contains amino acid modifications as compared to a parental Fc domain. Thus, a “variant human IgG1 Fc domain” is one that contains amino acid modifications (generally amino acid substitutions, although in the case of ablation variants, amino acid deletions are included) as compared to the human IgG1 Fc domain. In general, variant Fc domains have at least about 80, about 85, about 90, about 95, about 97, about 98 or about 99 percent identity to the corresponding parental human IgG Fc domain (using the identity algorithms discussed below, with one embodiment utilizing the BLAST algorithm as is known in the art, using default parameters). Alternatively, the variant Fc domains can have from 1 to about 20 (e.g., 1, 2, 3, 4, 5, 6, 7, 8, 9, 10, 11, 12, 13, 14, 15, 16, 17, 18, 19 or 20) amino acid modifications as compared to the parental Fc domain. Additionally, as discussed herein, the variant Fc domains herein still retain the ability to form a dimer with another Fc domain as measured using known techniques as described herein, such as non-denaturing gel electrophoresis.

By “heavy chain constant region” herein is meant the CH1-hinge-CH2-CH3 portion of an antibody (or fragments thereof), excluding the variable heavy domain; in EU numbering of human IgG1 this is amino acids 118-447 By “heavy chain constant region fragment” herein is meant a heavy chain constant region that contains fewer amino acids from either or both of the N- and C-termini but still retains the ability to form a dimer with another heavy chain constant region.

By “variable region” or “variable domain” as used herein is meant the region of an immunoglobulin that comprises one or more Ig domains substantially encoded by any of the V_{κ}, Vλ, and/or VH genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (vhCDR1, vhCDR2 and vhCDR3 for the variable heavy domain and vlCDR1, vlCDR2 and vlCDR3 for the variable light domain) and four framework (FR) regions, arranged from amino-terminus to carboxy-terminus in the following order: FR1-CDR1-FR2-CDR2-FR3-CDR3-FR4.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein is meant an amino acid modification that is not isotypic. For example, because none of the human IgGs comprise a serine at position 434, the substitution 434S in IgG1, IgG2, IgG3, or IgG4 (or hybrids thereof) is considered a non-naturally occurring modification.

The antibodies and antigen-binding fragments thereof of the disclosure are recombinant antibodies that have been engineered to have the various properties described herein and are generally isolated prior to use. As used herein, the term “isolated”, when used to describe the various polypeptides described herein, refers to a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogenous host cells, and they can be isolated as well.

As used herein, a “3E10 antibody” refers to an antibody with a set of heavy chain CDRs (VH CDR1, VH CDR2, and VH CDR3), identified according to the Kabat system, comprising amino acid sequences that vary from SEQ ID NOS: 58, 59, and 60 by no more than two amino acids each, respectively, a set of light chain CDRs (VL CDR1, VL CDR2, and VL CRD3) comprising amino acid sequences that vary from SEQ ID NOs: 61, 62, and 63 by no more than two amino acids each, respectively, and that binds nucleic acids. As described herein, the 3E10 antigen is a polynucleotide

As used herein, the term “cell-penetrating” refers to an antibody or antigen binding fragment thereof that can penetrate a cell, e.g., a mammalian cell, without the aid of an exogeneous transport vehicle, such as a liposome, or a conjugated cell-penetrating peptide. With respect to 3E10 antibodies and antigen binding fragments thereof, the cell-penetrating antibody or antigen binding fragment thereof can penetrate a cell expressing an ENT2 receptor on its cell surface in the presence of nucleic acids, e.g., non-covalently bound and/or conjugated to the 3E10 antibody or antigen binding fragment thereof, resulting in internalization of the 3E10 antibodies and antigen binding fragments thereof. In some embodiments, the cell-penetrating 3E10 antibody or antigen binding fragment thereof is conjugated to a functional molecule, e.g., a chemical agent, polynucleotide, or polypeptide

By “skeletal muscle polypeptide” herein is meant a polypeptide having a substantially similar structure and function as a protein, or polypeptide chain thereof, that is genetically-linked to a skeletal muscle disease, e.g., a protein, or polypeptide chain thereof, for which mutations exist that result in a skeletal muscle disease. The term “skeletal muscle polypeptide” encompasses wild type versions of skeletal muscle proteins, and polypeptide chains thereof, natural variant versions of skeletal muscle proteins, and polypeptide chains thereof, as well as engineered versions of skeletal muscle proteins, and polypeptide chains thereof. Skeletal muscle polypeptides are also intended to encompass proteins, and polypeptide chains thereof, having a function that partially or completely rescues a function lost by a mutation in a protein, or polypeptide chain thereof, genetically-linked to a skeletal muscle disease, including but not limited to various homologues of a skeletal muscle protein, or polypeptide chain thereof.

By “modification” herein is meant an amino acid substitution, insertion, and/or deletion in a polypeptide sequence.

By “variant protein” or “protein variant”, or “variant” as used herein is meant a protein that differs from that of a parent protein by virtue of at least one amino acid modification. The protein variant has at least one amino acid modification compared to the parent protein, yet not so many that the variant protein will not align with the parental protein using an alignment program such as that described below. In general, variant proteins (such as variant Fc domains, etc., outlined herein, are generally at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the parent protein, using the alignment programs described below, such as BLAST. Although amino acid sequence modifications to a 3E10 antibody or antigen binding fragment thereof as described herein may produce a protein and/or polypeptide that is referred to as a variant 3E10 antibody or antigen binding fragment thereof, such variants still fall within the classification of a 3E10 antibody or antigen binding fragment thereof as long as they maintain the CDR sequence and cell penetrating activity requirements of a 3E10 antibody or antigen binding fragment thereof.

Sequence identity between two similar sequences (e.g., antibody variable domains) can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, CD. (1970) “A General Method Applicable To The Search For Similarities In The Amino Acid Sequence Of Two Proteins,” J. Mol. Biol., 48:443 [homology alignment algorithm], Pearson, W. R. & Lipman, D. J. (1988) “Improved Tools For Biological Sequence Comparison,” Proc. Natl. Acad. Sci. USA 85:2444 [search for similarity method]; or Altschul, S. F. et al, (1990) “Basic Local Alignment Search Tool,” J. Mol. Biol. 215:403-10, the “BLAST” algorithm, see the webpage located at URL blast.ncbi.nlm.nih.gov/Blast.cgi. When using any of the aforementioned algorithms, the default parameters (for Window length, gap penalty, etc.) are used. Unless specifically stated otherwise, sequence identity is determined using the BLAST algorithm, using default parameters

As used herein, the term “subject” means any individual who is the target of administration. The subject can be a vertebrate, for example, a mammal. Thus, the subject can be a human. The term does not denote a particular age or sex.

As used herein, the term “pharmaceutically effective amount” means that the amount of the composition used is of sufficient quantity to ameliorate one or more causes or symptoms of a disease or disorder. Such amelioration only requires a reduction or alteration, not necessarily elimination. The precise dosage will vary according to a variety of factors such as subject-dependent variables (e.g., age, immune system health, etc.), the disease or disorder being treated, as well as the route of administration and the pharmacokinetics of the agent being administered.

As used herein, the term “carrier” or “excipient” refers to an organic or inorganic ingredient, natural or synthetic inactive ingredient in a formulation, with which one or more active ingredients are combined. The carrier or excipient would naturally be selected to minimize degradation of the active ingredient or to minimize adverse side effects in the subject, as would be well known to one of skill in the art.

As used herein, the term “treat” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

### 3E10 Antibodies, Variants, and Fragments Thereof

In some aspects, the present disclosure relates to the use of 3E10 antibodies, and derivatives thereof, for delivering therapeutic mRNA molecules to skeletal muscle tissue in a subject, e.g., to treat a genetic skeletal muscle disease. As is discussed below, the term antibody is used generally. Antibodies that find use in the present disclosure take on a number of formats as described herein, including traditional antibodies as well as antibody derivatives, fragments, and mimetics, described herein in various embodiments.

Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. The present disclosure is directed to antibodies that generally are based on the IgG class, which has several subclasses, including, but not limited to IgG1, IgG2, IgG3, and IgG4. In general, IgG1, IgG2 and IgG4 are used more frequently than IgG3. It should be noted that IgG1 has different allotypes with polymorphisms at 356 (D or E) and 358 (L or M).

The light chain generally comprises two domains, the variable light domain (containing the light chain CDRs and together with the variable heavy domains forming the Fv region), and a constant light chain region (often referred to as CL or C_{κ}). The heavy chain comprises a variable heavy domain and a constant domain, which includes a CH1-optional hinge-Fc domain comprising a CH2-CH3.

The hypervariable region of an antibody generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs useful for the compositions and methods described herein are described below.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g. vhCDR1, vhCDR2 and vhCDR3) and the disclosure of each variable light region is a disclosure of the vlCDRs (e.g. vlCDR1, vlCDR2 and vlCDR3). A useful comparison of CDR numbering is described in Lafranc et al., Dev. Comp. Immunol. 27 (1): 55-77 (2003).

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

The present disclosure provides a large number of different CDR sets. In this case, a “full CDR set” comprises the three variable light and three variable heavy CDRs, e.g. a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as nucleic acids, amino acids, or sugar side chains and usually have specific structural characteristics, as well as specific charge characteristics. A single antigen may have more than one epitope. The antibodies described herein bind to nucleic acid epitopes in a partially sequence-independent manner. That is, while the antibodies described herein bind to some polynucleotide structures and sequences with greater affinity than other nucleic acid structures and sequences, they have some general affinity for polynucleotides.

The “Fc domain” of the heavy chain includes the —CH2-CH3 domain, and optionally a hinge domain (—H—CH2-CH3). For IgG, the Fc domain comprises immunoglobulin domains CH2 and CH3 (Cγ2 and Cγ3) and the lower hinge region between CH1 (Cγ1) and CH2 (Cγ2). Although the boundaries of the Fc region may vary, the human IgG heavy chain Fc region is usually defined to include residues C226 or P230 to its carboxyl-terminus, wherein the numbering is according to the EU index as in Kabat. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. Thus, the “Fc domain” includes the —CH2-CH3 domain, and optionally a hinge domain (hinge-CH2-CH3). In the embodiments herein, when a scFv is attached to an Fc domain, it is generally the C-terminus of the scFv construct that is attached to all or part of the hinge of the Fc domain; for example, it is generally attached to the sequence EPKS which is the beginning of the hinge. In some embodiments, as is more fully described below, amino acid modifications are made to the Fc region, for example to alter binding to one or more FcγR receptors or to the FcRn receptor, and to enable heterodimer formation and purification, as outlined herein.

Another part of the heavy chain is the hinge region. By “hinge” or “hinge region” or “antibody hinge region” or “hinge domain” herein is meant the flexible polypeptide comprising the amino acids between the first and second constant domains of an antibody. Structurally, the IgG CH1 domain ends at EU position 215, and the IgG CH2 domain begins at residue EU position 231. Thus for IgG the antibody hinge is herein defined to include positions 216 (E216 in IgG1) to 230 (p230 in IgG1), wherein the numbering is according to the EU index as in Kabat. In some cases, a “hinge fragment” is used, which contains fewer amino acids at either or both of the N- and C-termini of the hinge domain.

A scFv comprises a variable heavy chain, an scFv linker, and a variable light domain. In most of the constructs and sequences outlined herein, the C-terminus of the variable heavy chain is attached to the N-terminus of the scFv linker, the C-terminus of which is attached to the N-terminus of a variable light chain (N-vh-linker-vl-C) although that can be switched (N-vl-linker-vh-C).

Thus, the present disclosure relates to different antibody domains. As described herein and known in the art, the heterodimeric antibodies described in certain embodiments of the disclosure comprise different domains within the heavy and light chains, which can be overlapping as well. These domains include, but are not limited to, the Fc domain, the CH1 domain, the CH2 domain, the CH3 domain, the hinge domain, the heavy constant domain (CH1-hinge-Fc domain or CH1-hinge-CH2-CH3), the variable heavy domain, the variable light domain, the light constant domain, Fab domains and scFv domains.

In certain embodiments, the antibodies of the disclosure comprise a heavy chain variable region from a particular germline heavy chain immunoglobulin gene and/or a light chain variable region from a particular germline light chain immunoglobulin gene. For example, such antibodies may comprise or consist of a human antibody comprising heavy or light chain variable regions that are “the product of” or “derived from” a particular germline sequence, e.g., that of the 3E10 antibody. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody (using the methods outlined herein). A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the germline immunoglobulin gene. Typically, a humanized antibody derived from a particular human germline sequence will display no more than 10-20 amino acid differences from the amino acid sequence encoded by the human germline immunoglobulin gene. In certain cases, the humanized antibody may display no more than 5, or even no more than 4, 3, 2, or 1 amino acid difference from the amino acid sequence encoded by the germline immunoglobulin gene.

In one embodiment, the parent antibody has been affinity matured, as is known in the art. Structure-based methods may be employed for humanization and affinity maturation, for example as described in U.S. Ser. No. 11/004,590, which is incorporated herein by reference. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272 (16): 10678-10684, Rosok et al., 1996, J. Biol. Chem. 271 (37): 22611-22618; Rader et al., 1998, Proc. Natl. Acad. Sci. USA 95:8910-8915; Krauss et al., 2003, Protein Engineering 16 (10): 753-759, all of which are incorporated herein by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all of which are incorporated herein by reference.

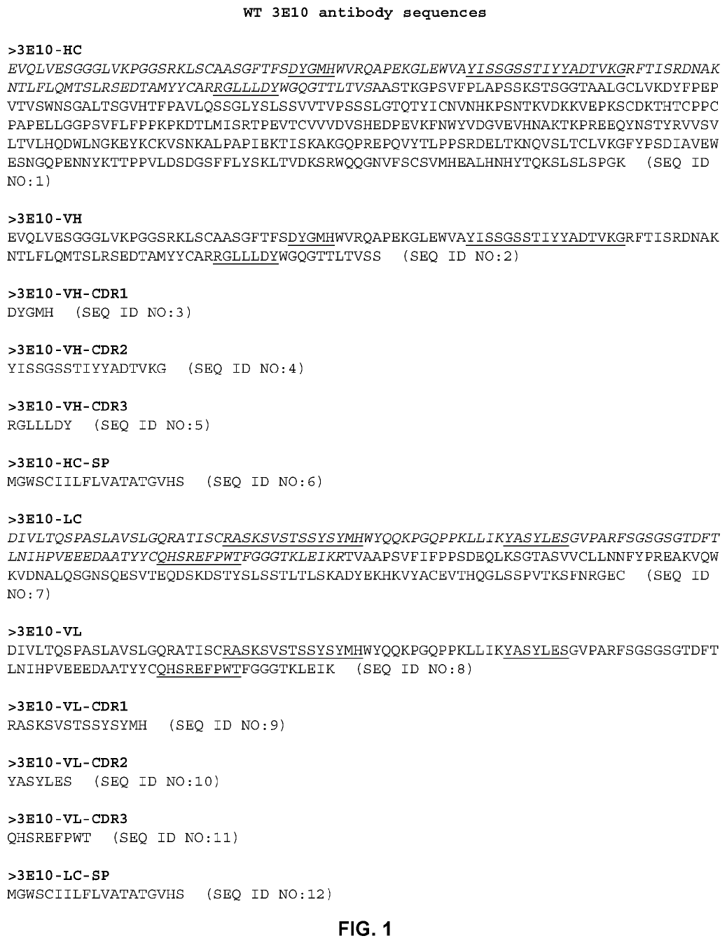

In some aspects, the disclosure relates to the use of antigen binding domains (ABDs) that bind to nucleic acids, and specifically that bind to therapeutic polynucleotides, derived from the 3E10 antibody. The amino acid sequence of the heavy and light chains of the parent 3E10 antibody are shown in FIG. 1. Accordingly, in some embodiments, the compositions described herein include a 3E10 antibody or antigen-binding fragment thereof.

In some embodiments, a 3E10 antibody or antigen-binding fragment thereof described herein includes CDR sequences corresponding to the parent 3E10 antibody, shown in FIG. 1. Accordingly, in some embodiments, the a 3F10 antibody or antigen-binding fragment thereof includes a light chain variable region (VL) complementarity determining region (CDR) 1 comprising the amino acid sequence of 3E10-VL-CDR1 (SEQ ID NO: 9), a VL CDR2 comprising the amino acid sequence of 3E10-VL-CDR2 (SEQ ID NO: 10), a VL CDR3 comprising the amino acid sequence of 3E10-VL-CDR3 (SEQ ID NO: 11), a heavy chain variable region (VH) CDR1 comprising the amino acid sequence of 3E10-VH-CDR1 (SEQ ID NO: 3), a VH CDR2 comprising the amino acid sequence of 3E10-VH-CDR2 (SEQ ID NO: 4), and a VH CDR3 comprising the amino acid sequence of 3E10-VH-CDR3 (SEQ ID NO: 5).

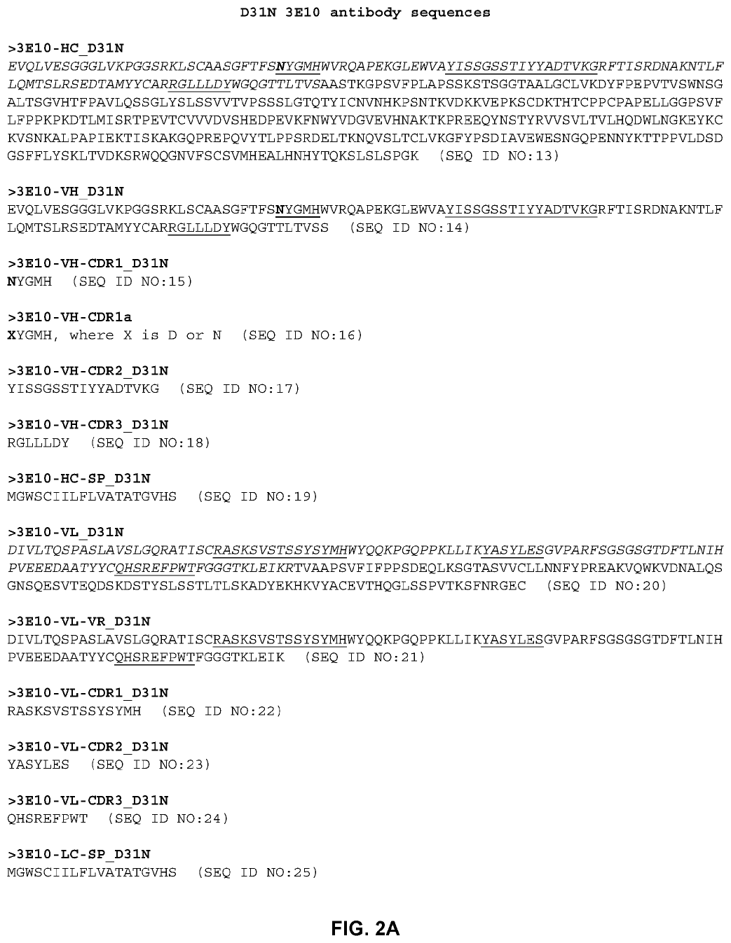

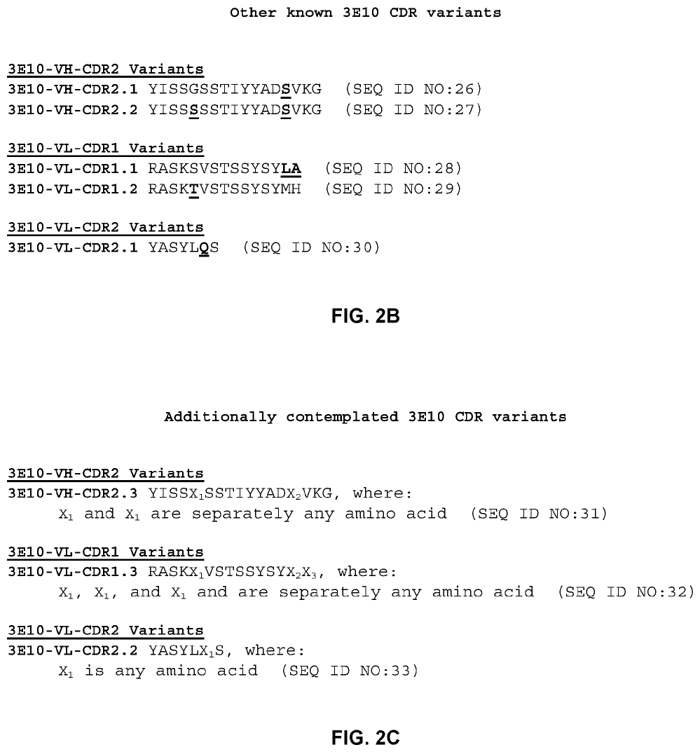

In some embodiments, a 3E10 antibody or antigen-binding fragment thereof described herein includes CDR sequences from a variant 3E10 antibody that includes a D3IN amino acid substitution in the VH CDR1, as shown in FIG. 2. Accordingly, in some embodiments, the a 3E10 antibody or antigen-binding fragment thereof includes a light chain variable region (VL) complementarity determining region (CDR) 1 comprising the amino acid sequence of 3E10-VL-CDR1_D31N (SEQ ID NO: 22), a VL CDR2 comprising the amino acid sequence of 3E10-VL-CDR2_D3IN (SEQ ID NO: 23), a VL CDR3 comprising the amino acid sequence of 3E10-VL-CDR3_D31N (SEQ ID NO: 24), a heavy chain variable region (VH) CDR1 comprising the amino acid sequence of 3E10-VH-CDR1_D31N (SEQ ID NO: 15), a VH CDR2 comprising the amino acid sequence of 3E10-VH-CDR2_D31N (SEQ ID NO: 17), and a VH CDR3 comprising the amino acid sequence of 3E10-VH-CDR3_D3IN (SEQ ID NO: 18).

In some embodiments, a 3E10 antibody or antigen-binding fragment thereof described herein refers to CDR sequences corresponding to the parent 3E10 antibody, shown in FIG. 1, optionally including a D31N amino acid substitution in the VH CDR1. Accordingly, in some embodiments, a 3E10 antibody or antigen-binding fragment thereof includes a light chain variable region (VL) complementarity determining region (CDR) 1 comprising the amino acid sequence of 3E10-VL-CDR1 (SEQ ID NO: 9), a VL CDR2 comprising the amino acid sequence of 3E10-VL-CDR2 (SEQ ID NO: 10), a VL CDR3 comprising the amino acid sequence of 3E10-VL-CDR3 (SEQ ID NO: 11), a heavy chain variable region (VH) CDR1 comprising the amino acid sequence of 3E10-VH-CDR1a (SEQ ID NO: 16), a VH CDR2 comprising the amino acid sequence of 3E10-VH-CDR2 (SEQ ID NO: 4), and a VH CDR3 comprising the amino acid sequence of 3E10-VH-CDR3 (SEQ ID NO: 5).

In some embodiments, a 3E10 antibody or antigen-binding fragment thereof described herein includes CDR sequences corresponding to the parent 3E10 antibody, shown in FIG. 1, with a known amino acid substitution in one or more CDR. For example, FIG. 2B shows the amino acid sequence of several known VH CDR2, VL CDR1, and VL CDR2 amino acid sequences. Accordingly, in some embodiments, a 3E10 antibody or antigen-binding fragment thereof described herein includes one or more amino acid substitution, relative to the CDR sequences of the parent 3E10 (shown in FIG. 1) or 3E10-D3IN variant (shown in FIG. 2), selected from a G to S substitution at position 5 of VH CDR2, a T to S substitution at position 14 of VH CDR2, an S to T substitution at position 5 of VL CDR1, an M to L substitution at position 14 of VL CDR1, an H to A substitution at position 15 of VL CDR1, and an E to Q substitution at position 6 of VL CDR2.

Accordingly, in some embodiments, a 3E10 antibody or antigen-binding fragment thereof includes VH CDR2 comprising the amino acid sequence of 3E10-VH-CDR2.1 (SEQ ID NO: 26) or 3E10-VH-CDR2.2 (SEQ ID NO: 27). In some embodiments, the 3E10 antibody or antigen-binding fragment thereof further includes VL CDRs 1-3, and VH CDRs 1 and 3 according to the parent 3E10 antibody (as shown in FIG. 1). In some embodiments, the 3E10 antibody or antigen-binding fragment thereof further includes VL CDRs 1-3, and VH CDRs 1 and 3 according to the 3E10-D3IN variant (as shown in FIG. 2A). In some embodiments, the 3E10 antibody or antigen-binding fragment thereof further includes VL CDRs 1-3, and VH CDRs 1 and 3 having one or more amino acid substitutions relative to the CDRs of the parent 3E10 antibody (as shown in FIG. 1) or relative to the 3E10-D3IN variant (as shown in FIG. 2A).

Similarly, in some embodiments, a 3E10 antibody or antigen-binding fragment thereof includes VL CDR1 comprising the amino acid sequence of 3E10-VL-CDR1.1 (SEQ ID NO: 28) or 3E10-VL-CDR1.2 (SEQ ID NO: 29). In some embodiments, the 3E10 antibody or antigen-binding fragment thereof further includes VL CDRs 2 and 3, and VH CDRs 1-3 according to the parent 3E10 antibody (as shown in FIG. 1). In some embodiments, the 3E10 antibody or antigen-binding fragment thereof further includes VL CDRs 2 and 3, and VH CDRs 1-3 according to the 3E10-D3IN variant (as shown in FIG. 2A). In some embodiments, the 3E10 antibody or antigen-binding fragment thereof further includes VL CDRs 2 and 3, and VH CDRs 1-3 having one or more amino acid substitutions relative to the CDRs of the parent 3E10 antibody (as shown in FIG. 1) or relative to the 3E10-D3IN variant (as shown in FIG. 2A).

Similarly, in some embodiments, a 3E10 antibody or antigen-binding fragment thereof includes VL CDR2 comprising the amino acid sequence of 3E10-VL-CDR2.1 (SEQ ID NO: 30). In some embodiments, the 3E10 antibody or antigen-binding fragment thereof further includes VL CDRs 1 and 3, and VH CDRs 1-3 according to the parent 3E10 antibody (as shown in FIG. 1). In some embodiments, the 3E10 antibody or antigen-binding fragment thereof further includes VL CDRs 1 and 3, and VH CDRs 1-3 according to the 3E10-D3IN variant (as shown in FIG. 2A). In some embodiments, the 3E10 antibody or antigen-binding fragment thereof further includes VL CDRs 1 and 3, and VH CDRs 1-3 having one or more amino acid substitutions relative to the CDRs of the parent 3E10 antibody (as shown in FIG. 1) or relative to the 3E10-D31N variant (as shown in FIG. 2A).

While some of the amino acid substitutions described above are fairly conservative substitutions—e.g., an S to T substitution at position 5 of VL CDR1—other substitutions are to amino acids that have vastly different properties—e.g., an M to L substitution at position 14 of VL CDR1, an H to A substitution at position 15 of VL CDR1, and an E to Q substitution at position 6 of VL CDR2. This suggests, without being bound by theory, that at least these positions within the 3E10 CDR framework are tolerant to other amino acid substitutions.

Accordingly, in some embodiments, a 3E10 antibody or antigen-binding fragment thereof includes VH CDR2 comprising the amino acid sequence of 3E10-VH-CDR2.3 (SEQ ID NO: 31). In some embodiments, the 3E10 antibody or antigen-binding fragment thereof further includes VL CDRs 1-3, and VH CDRs 1 and 3 according to the parent 3E10 antibody (as shown in FIG. 1). In some embodiments, the 3E10 antibody or antigen-binding fragment thereof further includes VL CDRs 1-3, and VH CDRs 1 and 3 according to the 3E10-D3IN variant (as shown in FIG. 2A). In some embodiments, the 3E10 antibody or antigen-binding fragment thereof further includes VL CDRs 1-3, and VH CDRs 1 and 3 having one or more amino acid substitutions relative to the CDRs of the parent 3E10 antibody (as shown in FIG. 1) or relative to the 3E10-D3IN variant (as shown in FIG. 2A), e.g., as described herein.

Similarly, in some embodiments, a 3E10 antibody or antigen-binding fragment thereof includes VL CDR1 comprising the amino acid sequence of 3E10-VL-CDR1.3 (SEQ ID NO: 32). In some embodiments, the 3E10 antibody or antigen-binding fragment thereof further includes VL CDRs 2 and 3, and VH CDRs 1-3 according to the parent 3E10 antibody (as shown in FIG. 1). In some embodiments, the 3E10 antibody or antigen-binding fragment thereof further includes VL CDRs 2 and 3, and VH CDRs 1-3 according to the 3E10-D3IN variant (as shown in FIG. 2A). In some embodiments, the 3E10 antibody or antigen-binding fragment thereof further includes VL CDRs 2 and 3, and VH CDRs 1-3 having one or more amino acid substitutions relative to the CDRs of the parent 3E10 antibody (as shown in FIG. 1) or relative to the 3E10-D3IN variant (as shown in FIG. 2A), e.g., as described herein.

Similarly, in some embodiments, a 3E10 antibody or antigen-binding fragment thereof, includes VL CDR2 comprising the amino acid sequence of 3E10-VL-CDR2.2 (SEQ ID NO: 33). In some embodiments, the 3E10 antibody or antigen-binding fragment thereof further includes VL CDRs 1 and 3, and VH CDRs 1-3 according to the parent 3E10 antibody (as shown in FIG. 1). In some embodiments, the 3E10 antibody or antigen-binding fragment thereof further includes VL CDRs 1 and 3, and VH CDRs 1-3 according to the 3E10-D3IN variant (as shown in FIG. 2A). In some embodiments, the 3E10 antibody or antigen-binding fragment thereof further includes VL CDRs 1 and 3, and VH CDRs 1-3 having one or more amino acid substitutions relative to the CDRs of the parent 3E10 antibody (as shown in FIG. 1) or relative to the 3E10-D31N variant (as shown in FIG. 2A), e.g., as described herein.

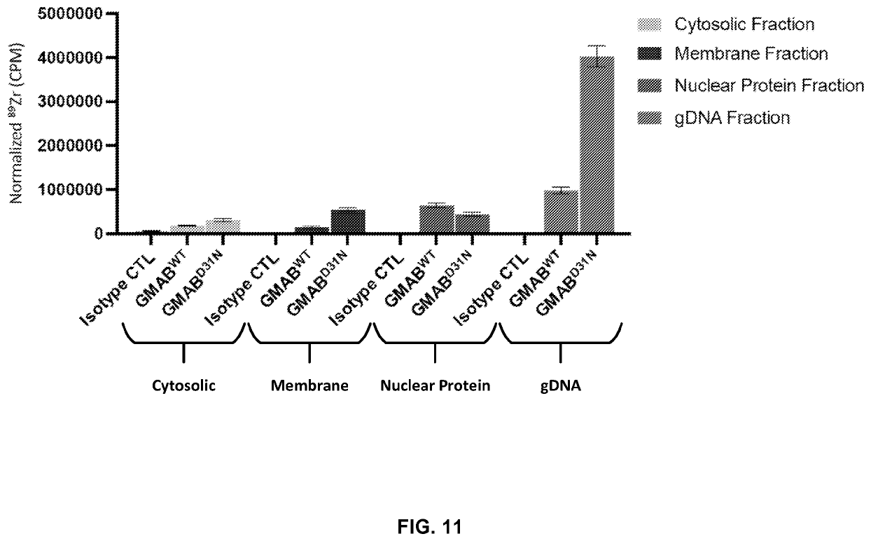

Further, because 3E10 antibodies or variants thereof, or antigen-binding fragments thereof, bind to nucleic acid in a partially sequence-independent manner, and without being bound by theory, it was contemplated that the interaction may be mediated by electrostatic interactions with the nucleotide backbone. To investigate this theory, electrostatic surface potential renderings of a molecular model of a 3E10-scFv construct—the amino acid sequence of which is illustrated in FIG. 11C-were generated, as shown in FIGS. 11A and 11B. These models revealed a putative Nucleic Acid Binding pocket (NAB1) corresponding to a large basic region on the surface of the molecule, as illustrated in FIG. 11A. The position of the non-hydrogen atoms of the amino acids contributing to the putative Nucleic Acid Binding pocket in the model are superposed in FIG. 11B, and the amino acid residues are mapped onto the sequence of the construct in FIG. 11C.

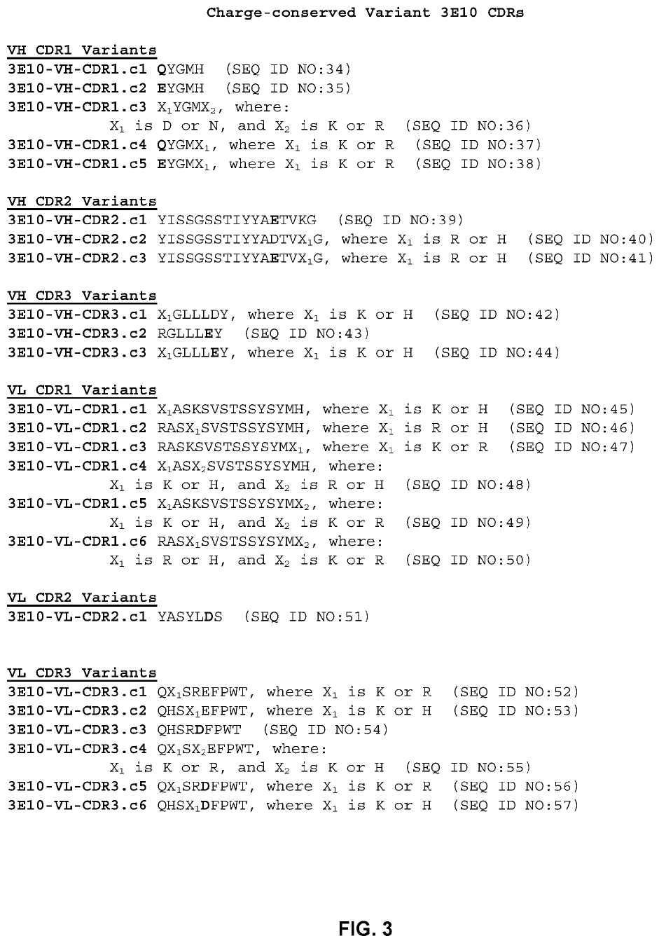

Thus, it is contemplated that amino acid substitutions within the CDRs of a 3E10 antibody or antigen-binding fragment thereof, as described herein, that maintain the electrostatic character of this putative Nucleic Acid Binding pocket will also retain the nucleic acid binding properties of the construct. Accordingly, in some embodiments, a 3E10 antibody or antigen-binding fragment thereof, includes one or more amino acid substitution of a first basic amino acid to a second basic amino acid (e.g., K, R, or H). Similarly, in some embodiments, a 3E10 antibody or antigen-binding fragment thereof, includes one or more amino acid substitution of a first acidic amino acid to a second acidic amino acid (e.g., D or E). Examples of such charge-conserved variant 3E10 CDRs are shown in FIG. 3.

Accordingly, in some embodiments, a 3E10 antibody or antigen-binding fragment thereof includes VH CDR1 comprising the amino acid sequence of 3E10-VH-CDR1.c1 (SEQ ID NO: 34), 3E10-VH-CDR1.c2 (SEQ ID NO: 35), 3E10-VH-CDR1.c3 (SEQ ID NO: 36), 3E10-VH-CDR1.c4 (SEQ ID NO: 37), or 3E10-VH-CDR1.c5 (SEQ ID NO: 38). In some embodiments, the 3E10 antibody or antigen-binding fragment thereof further includes VL CDRs 1-3, and VH CDRs 2 and 3 according to the parent 3E10 antibody (as shown in FIG. 1). In some embodiments, the 3E10 antibody or antigen-binding fragment thereof further includes VL CDRs 1-3, and VH CDRs 1 and 3 having one or more amino acid substitutions relative to the CDRs of the parent 3E10 antibody (as shown in FIG. 1), e.g., as described herein.

Similarly, in some embodiments, a 3E10 antibody or antigen-binding fragment thereof includes VH CDR2 comprising the amino acid sequence of 3E10-VH-CDR2.c1 (SEQ ID NO: 39), 3E10-VH-CDR2.c2 (SEQ ID NO: 40), or 3E10-VH-CDR2.c3 (SEQ ID NO: 41). In some embodiments, the 3E10 antibody or antigen-binding fragment thereof further includes VL CDRs 1-3, and VH CDRs 1 and 3 according to the parent 3E10 antibody (as shown in FIG. 1). In some embodiments, the 3E10 antibody or antigen-binding fragment thereof further includes VL CDRs 1-3, and VH CDRs 1 and 3 according to the 3E10-D3IN variant (as shown in FIG. 2A). In some embodiments, the 3E10 antibody or antigen-binding fragment thereof further includes VL CDRs 1-3, and VH CDRs 1 and 3 having one or more amino acid substitutions relative to the CDRs of the parent 3E10 antibody (as shown in FIG. 1), e.g., as described herein.

Similarly, in some embodiments, a 3E10 antibody or antigen-binding fragment thereof includes VH CDR3 comprising the amino acid sequence of 3E10-VH-CDR3.c1 (SEQ ID NO: 42), 3E10-VH-CDR3.c2 (SEQ ID NO: 43), or 3E10-VH-CDR3.c3 (SEQ ID NO: 44). In some embodiments, the 3E10 antibody or antigen-binding fragment thereof further includes VL CDRs 1-3, and VH CDRs 1 and 2 according to the parent 3E10 antibody (as shown in FIG. 1). In some embodiments, the 3E10 antibody or antigen-binding fragment thereof further includes VL CDRs 1-3, and VH CDRs 1 and 2 according to the 3E10-D3IN variant (as shown in FIG. 2A). In some embodiments, the 3E10 antibody or antigen-binding fragment thereof further includes VL CDRs 1-3, and VH CDRs 1 and 2 having one or more amino acid substitutions relative to the CDRs of the parent 3E10 antibody (as shown in FIG. 1), e.g., as described herein.

Similarly, in some embodiments, a 3E10 antibody or antigen-binding fragment thereof includes VL CDR1 comprising the amino acid sequence of 3E10-VL-CDR1.c1 (SEQ ID NO: 45), 3E10-VL-CDR1.c2 (SEQ ID NO: 46), 3E10-VL-CDR1.c3 (SEQ ID NO: 47), 3E10-VL-CDR1.c4 (SEQ ID NO: 48), 3E10-VL-CDR1.c5 (SEQ ID NO: 49), or 3E10-VL-CDR1.c6 (SEQ ID NO: 50). In some embodiments, the 3E10 antibody or antigen-binding fragment thereof further includes VL CDRs 2 and 3, and VH CDRs 1-3 according to the parent 3E10 antibody (as shown in FIG. 1). In some embodiments, the 3E10 antibody or antigen-binding fragment thereof further includes VL CDRs 2 and 3, and VH CDRs 1-3 according to the 3E10-D3IN variant (as shown in FIG. 2A). In some embodiments, the 3E10 antibody or antigen-binding fragment thereof further includes VL CDRs 2 and 3, and VH CDRs 1-3 having one or more amino acid substitutions relative to the CDRs of the parent 3E10 antibody (as shown in FIG. 1), e.g., as described herein.

Similarly, in some embodiments, a 3E10 antibody or antigen-binding fragment thereof includes VL CDR2 comprising the amino acid sequence of 3E10-VL-CDR2.c1 (SEQ ID NO: 51). In some embodiments, the 3E10 antibody or antigen-binding fragment thereof further includes VL CDRs 1 and 3, and VH CDRs 1-3 according to the parent 3E10 antibody (as shown in FIG. 1). In some embodiments, the 3E10 antibody or antigen-binding fragment thereof further includes VL CDRs 1 and 3, and VH CDRs 1-3 according to the 3E10-D3IN variant (as shown in FIG. 2A). In some embodiments, the 3F10 antibody or antigen-binding fragment thereof further includes VL CDRs 1 and 3, and VH CDRs 1-3 having one or more amino acid substitutions relative to the CDRs of the parent 3E10 antibody (as shown in FIG. 1), e.g., as described herein.

Similarly, in some embodiments, a 3E10 antibody or antigen-binding fragment thereof includes VL CDR3 comprising the amino acid sequence of 3E10-VL-CDR3.c1 (SEQ ID NO: 52), 3E10-VL-CDR3.c2 (SEQ ID NO: 53), 3E10-VL-CDR3.c3 (SEQ ID NO: 54), 3E10-VL-CDR3.c4 (SEQ ID NO: 55), 3E10-VL-CDR3.c5 (SEQ ID NO: 56), or 3E10-VL-CDR3.c6 (SEQ ID NO: 57). In some embodiments, the 3E10 antibody or antigen-binding fragment thereof further includes VL CDRs 1 and 2, and VH CDRs 1-3 according to the parent 3E10 antibody (as shown in FIG. 1). In some embodiments, the 3E10 antibody or antigen-binding fragment thereof further includes VL CDRs 1 and 2, and VH CDRs 1-3 according to the 3E10-D3IN variant (as shown in FIG. 2A). In some embodiments, the 3E10 antibody or antigen-binding fragment thereof further includes VL CDRs 1 and 2, and VH CDRs 1-3 having one or more amino acid substitutions relative to the CDRs of the parent 3E10 antibody (as shown in FIG. 1), e.g., as described herein.

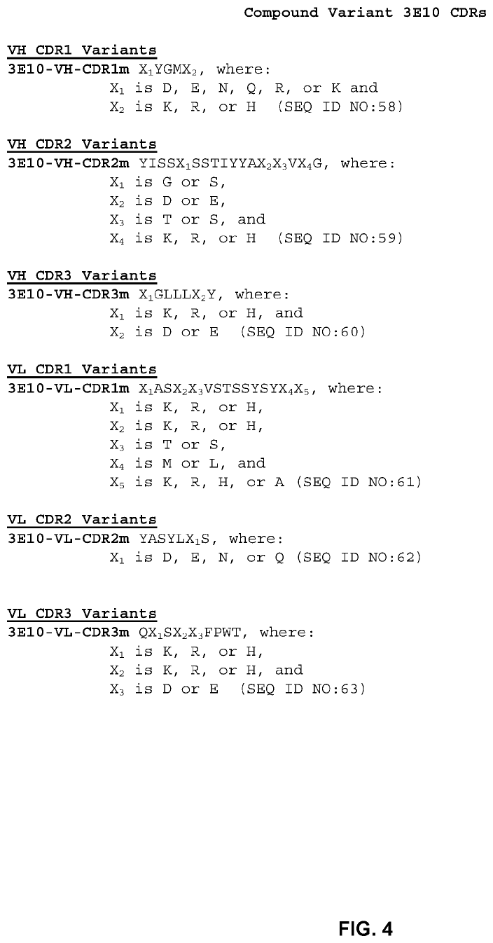

It is also contemplated that a 3E10 antibody or antigen-binding fragment thereof, as described herein, includes any combination of the 3E10 CDR amino acid substitutions described above. Examples of 3E10 variant CDR sequences that incorporate one or more of the amino acid substitutions described herein are shown in FIG. 4.

Accordingly, in some embodiments, a 3E10 antibody or antigen-binding fragment thereof includes VH CDR1 comprising the amino acid sequence of 3E10-VH-CDRIm (SEQ ID NO: 58). In some embodiments, the 3E10 antibody or antigen-binding fragment thereof further includes VL CDRs 1-3, and VH CDRs 2 and 3 according to the parent 3E10 antibody (as shown in FIG. 1). In some embodiments, the 3E10 antibody or antigen-binding fragment thereof further includes VL CDRs 1-3, and VH CDRs 1 and 3 having one or more amino acid substitutions relative to the CDRs of the parent 3E10 antibody (as shown in FIG. 1), e.g., as described herein.

Similarly, in some embodiments, a 3E10 antibody or antigen-binding fragment thereof includes VH CDR2 comprising the amino acid sequence of 3E10-VH-CDR2m (SEQ ID NO: 59). In some embodiments, the 3E10 antibody or antigen-binding fragment thereof further includes VL CDRs 1-3, and VH CDRs 1 and 3 according to the parent 3E10 antibody (as shown in FIG. 1). In some embodiments, the 3E10 antibody or antigen-binding fragment thereof further includes VL CDRs 1-3, and VH CDRs 1 and 3 according to the 3E10-D3IN variant (as shown in FIG. 2A). In some embodiments, the 3E10 antibody or antigen-binding fragment thereof further includes VL CDRs 1-3, and VH CDRs 1 and 3 having one or more amino acid substitutions relative to the CDRs of the parent 3E10 antibody (as shown in FIG. 1), e.g., as described herein.

Similarly, in some embodiments, a 3E10 antibody or antigen-binding fragment thereof includes VH CDR3 comprising the amino acid sequence of 3E10-VH-CDR3m (SEQ ID NO: 60). In some embodiments, the 3E10 antibody or antigen-binding fragment thereof further includes VL CDRs 1-3, and VH CDRs 1 and 2 according to the parent 3E10 antibody (as shown in FIG. 1). In some embodiments, the 3E10 antibody or antigen-binding fragment thereof further includes VL CDRs 1-3, and VH CDRs 1 and 2 according to the 3E10-D3IN variant (as shown in FIG. 2A). In some embodiments, the 3E10 antibody or antigen-binding fragment thereof further includes VL CDRs 1-3, and VH CDRs 1 and 2 having one or more amino acid substitutions relative to the CDRs of the parent 3E10 antibody (as shown in FIG. 1), e.g., as described herein.

Similarly, in some embodiments, a 3E10 antibody or antigen-binding fragment thereof includes VL CDR1 comprising the amino acid sequence of 3E10-VL-CDR1m (SEQ ID NO: 61). In some embodiments, the 3E10 antibody or antigen-binding fragment thereof further includes VL CDRs 2 and 3, and VH CDRs 1-3 according to the parent 3E10 antibody (as shown in FIG. 1). In some embodiments, the 3E10 antibody or antigen-binding fragment thereof further includes VL CDRs 2 and 3, and VH CDRs 1-3 according to the 3E10-D3IN variant (as shown in FIG. 2A). In some embodiments, the 3E10 antibody or antigen-binding fragment thereof further includes VL CDRs 2 and 3, and VH CDRs 1-3 having one or more amino acid substitutions relative to the CDRs of the parent 3E10 antibody (as shown in FIG. 1), e.g., as described herein.

Similarly, in some embodiments, a 3E10 antibody or antigen-binding fragment thereof includes VL CDR2 comprising the amino acid sequence of 3E10-VL-CDR2m (SEQ ID NO: 62). In some embodiments, the 3E10 antibody or antigen-binding fragment thereof further includes VL CDRs 1 and 3, and VH CDRs 1-3 according to the parent 3E10 antibody (as shown in FIG. 1). In some embodiments, the 3E10 antibody or antigen-binding fragment thereof further includes VL CDRs 1 and 3, and VH CDRs 1-3 according to the 3E10-D3IN variant (as shown in FIG. 2A). In some embodiments, the 3E10 antibody or antigen-binding fragment thereof further includes VL CDRs 1 and 3, and VH CDRs 1-3 having one or more amino acid substitutions relative to the CDRs of the parent 3E10 antibody (as shown in FIG. 1), e.g., as described herein.

Similarly, in some embodiments, a 3E10 antibody or antigen-binding fragment thereof includes VL CDR3 comprising the amino acid sequence of 3E10-VL-CDR3m (SEQ ID NO: 63). In some embodiments, the 3E10 antibody or antigen-binding fragment thereof further includes VL CDRs 1 and 2, and VH CDRs 1-3 according to the parent 3E10 antibody (as shown in FIG. 1). In some embodiments, the 3E10 antibody or antigen-binding fragment thereof further includes VL CDRs 1 and 2, and VH CDRs 1-3 according to the 3E10-D3IN variant (as shown in FIG. 2A). In some embodiments, the 3E10 antibody or antigen-binding fragment thereof further includes VL CDRs 1 and 2, and VH CDRs 1-3 having one or more amino acid substitutions relative to the CDRs of the parent 3E10 antibody (as shown in FIG. 1), e.g., as described herein.

In some embodiments, a 3E10 antibody or antigen-binding fragment thereof described herein includes a light chain variable region (VL) complementarity determining region (CDR) 1 comprising the amino acid sequence of 3E10-VL-CDR1m (SEQ ID NO: 61), a VL CDR2 comprising the amino acid sequence of 3E10-VL-CDR2m (SEQ ID NO: 62), a VL CDR3 comprising the amino acid sequence of 3E10-VL-CDR3m (SEQ ID NO: 63), a heavy chain variable region (VH) CDR1 comprising the amino acid sequence of 3E10-VH-CDR1m (SEQ ID NO: 58), a VH CDR2 comprising the amino acid sequence of 3E10-VH-CDR2m (SEQ ID NO: 59), and a VH CDR3 comprising the amino acid sequence of 3E10-VH-CDR3m (SEQ ID NO: 60).

In some embodiments, a 3E10 antibody or antigen-binding fragment thereof described herein refers to CDR sequences having no more than one amino acid substitution relative to the parent 3E10 antibody, shown in FIG. 1, optionally including a D3IN amino acid substitution in the VH CDR1. Accordingly, in some embodiments, a 3E10 antibody or antigen-binding fragment thereof includes a light chain variable region (VL) complementarity determining region (CDR) 1 comprising an amino acid sequence having no more than one amino acid substitution relative to 3E10-VL-CDR1 (SEQ ID NO: 9), a VL CDR2 comprising an amino acid sequence having no more than one amino acid substitution relative to 3E10-VL-CDR2 (SEQ ID NO: 10), a VL CDR3 comprising an amino acid sequence having no more than one amino acid substitution relative to 3E10-VL-CDR3 (SEQ ID NO: 11), a heavy chain variable region (VH) CDR1 comprising an amino acid sequence having no more than one amino acid substitution relative to 3E10-VH-CDR1a (SEQ ID NO: 16), a VH CDR2 comprising an amino acid sequence having no more than one amino acid substitution relative to 3E10-VH-CDR2 (SEQ ID NO: 4), and a VH CDR3 comprising an amino acid sequence having no more than one amino acid substitution relative to 3E10-VH-CDR3 (SEQ ID NO: 5).

In some embodiments, a 3E10 antibody or antigen-binding fragment thereof described herein refers to CDR sequences having no more than two amino acid substitution relative to the parent 3E10 antibody, shown in FIG. 1, optionally including a D3IN amino acid substitution in the VH CDR1. Accordingly, in some embodiments, a 3E10 antibody or antigen-binding fragment thereof includes a light chain variable region (VL) complementarity determining region (CDR) 1 comprising an amino acid sequence having no more than two amino acid substitutions relative to 3E10-VL-CDR1 (SEQ ID NO: 9), a VL CDR2 comprising an amino acid sequence having no more than two amino acid substitutions relative to 3E10-VL-CDR2 (SEQ ID NO: 10), a VL CDR3 comprising an amino acid sequence having no more than two amino acid substitutions relative to 3E10-VL-CDR3 (SEQ ID NO: 11), a heavy chain variable region (VH) CDR1 comprising an amino acid sequence having no more than two amino acid substitutions relative to 3E10-VH-CDR1a (SEQ ID NO: 16), a VH CDR2 comprising an amino acid sequence having no more than two amino acid substitutions relative to 3E10-VH-CDR2 (SEQ ID NO: 4), and a VH CDR3 comprising an amino acid sequence having no more than two amino acid substitutions relative to 3E10-VH-CDR3 (SEQ ID NO: 5).

Other variants of a 3E10 antibody or antigen-binding fragment thereof are also known in the art, as disclosed for example, in Zack, et al., J. Immunol., 157 (5): 2082-8 (1996). For example, amino acid position 31 of the heavy chain variable region of 3E10 has been determined to be influential in the ability of the antibody and fragments thereof to penetrate nuclei and bind to DNA (bolded in SEQ ID NOs: 1, 2, and 13). A D31N mutation (bolded in SEQ ID NOs: 2 and 13) in CDR1 penetrates nuclei and binds DNA with much greater efficiency than the original antibody (Zack, et al., Immunology and Cell Biology, 72:513-520 (1994), Weisbart, et al., J. Autoimmun., 11, 539-546 (1998); Weisbart, Int. J. Oncol., 25, 1867-1873 (2004)). In some embodiments, the antibody has the D31N substitution.

Although generally referred to herein as “antigen binding fragments” of a 3E10 antibody,” it will be appreciated that fragments and binding proteins, including antigen-binding fragments, variants, and fusion proteins such as scFv, di-scFv, tr-scFv, and other single chain variable fragments, and other cell-penetrating, nucleic acid transporting molecules disclosed herein are encompassed by the phrase are also expressly provided for use in compositions and methods disclosed herein. Thus, the antibodies and other binding proteins are also referred to herein as cell-penetrating.

A humanized 3E10 antibody or antigen binding fragment thereof is capable of being transported into the cytoplasm and/or nucleus of the cells without the aid of a carrier or conjugate. For example, the monoclonal antibody 3E10 and active fragments thereof that are transported in vivo to the nucleus of mammalian cells without cytotoxic effect are disclosed in U.S. Pat. Nos. 4,812,397 and 7,189,396 to Richard Weisbart.

In some embodiments, a humanized 3E10 antibody or antigen binding fragment thereof binds and/or inhibits Rad51. See, e.g., Turchick, et al., Nucleic Acids Res., 45 (20): 11782-11799 (2017), US 2021/0340280, and US 2021/033881, the contents of which are incorporated by reference herein, in its entirety

Humanized 3E10 antibodies and ENT2 binding fragments thereof that can be used in the in the compositions and methods described herein include whole immunoglobulin (i.e., an intact antibody) of any class, fragments thereof, and synthetic proteins containing at least the antigen binding variable domain of an antibody. The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies. Therefore, the humanized 3E10 antibodies and ENT2 binding fragments thereof typically contain at least the CDRs necessary to maintain DNA binding and/or interfere with DNA repair.

The 3E10 antibody is typically a monoclonal 3E10, or a variant, derivative, fragment, fusion, or humanized form thereof that binds the same or different epitope(s) as 3E10.

A deposit according to the terms of the Budapest Treaty of a hybridoma cell line producing monoclonal antibody 3E10 was received on Sep. 6, 2000, and accepted by, American Type Culture Collection (ATCC), 10801 University Blvd., Manassas, VA 20110-2209, USA, and given Patent Deposit Number PTA-2439.

Thus, the antibody may have the same or different epitope specificity as monoclonal antibody 3E10 produced by ATCC No. PTA 2439 hybridoma. The antibody can have the paratope of monoclonal antibody 3E10. The antibody can be a single chain variable fragment of 3E10, or a variant, e.g., a conservative variant thereof. For example, the antibody can be a single chain variable fragment of 3E10 (3E10 Fv), or a variant thereof.

Generally, a humanized antibody is the result of a process in which the sequence of a parental antibody from a non-human species is modified to increase the overall similarity of the parental antibody to human antibodies, while retaining antigen binding activity of the parental antibody. Generally, the process involves identifying a human antibody, sometimes referred to as a scaffold antibody, and then either (i) replacing amino acids in the parent (non-human) antibody with equivalent amino acids from the scaffold (human) antibody, e.g., framework amino acids having little to no effect on antigen binding or (ii) replacing amino acids in the scaffold (human) antibody with equivalent amino acids from the parent (non-human) antibody, e.g., CDRs and other amino acids with significant effects on antigen binding. Various methods for humanization are known in the art, including framework-homology-based humanization, germline humanization, complementary determining regions (CDR)-homology-based humanization, and specificity determining residues (SDR) grafting. For a review of these methods see, for example, Safdari Y. et al., Biotechnology and Genetic Engineering Reviews, 29:2, 175-86 (2013).

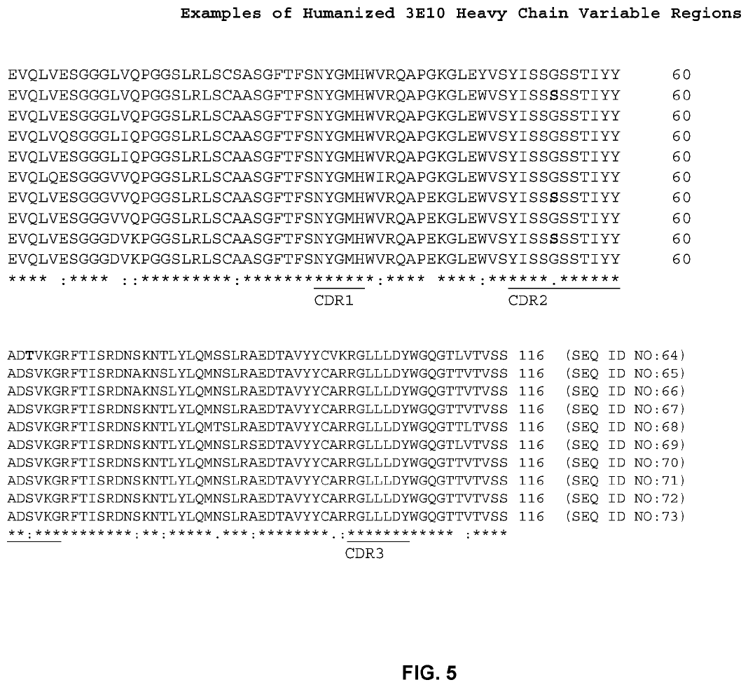

Exemplary 3F10 humanized sequences are discussed in WO 2015/106290, WO 2016/033324, WO 2019/018426, and WO/2019/018428, and provided in FIG. 5 (Humanized 3E10 Heavy Chain Variable Regions) and 6 (Humanized 3E10 Light Chain Variable Regions).

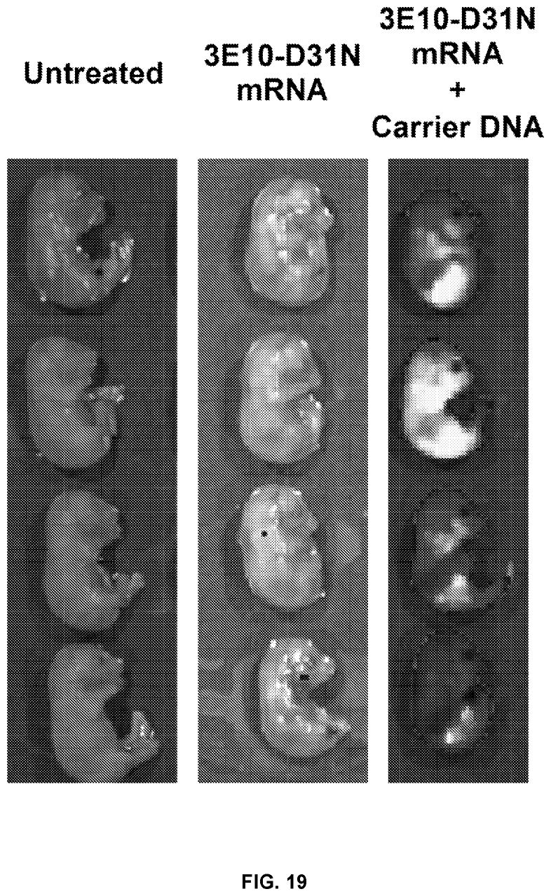

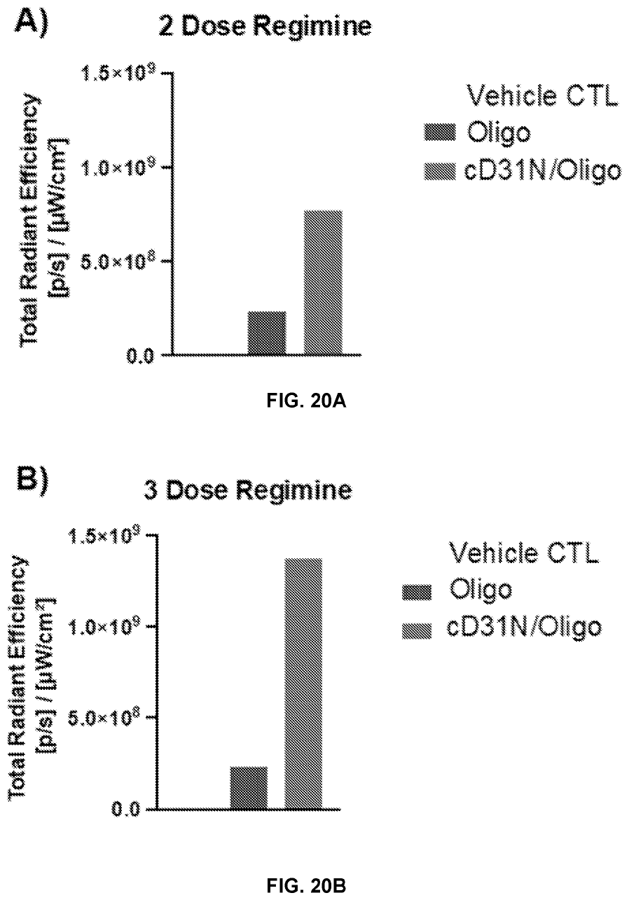

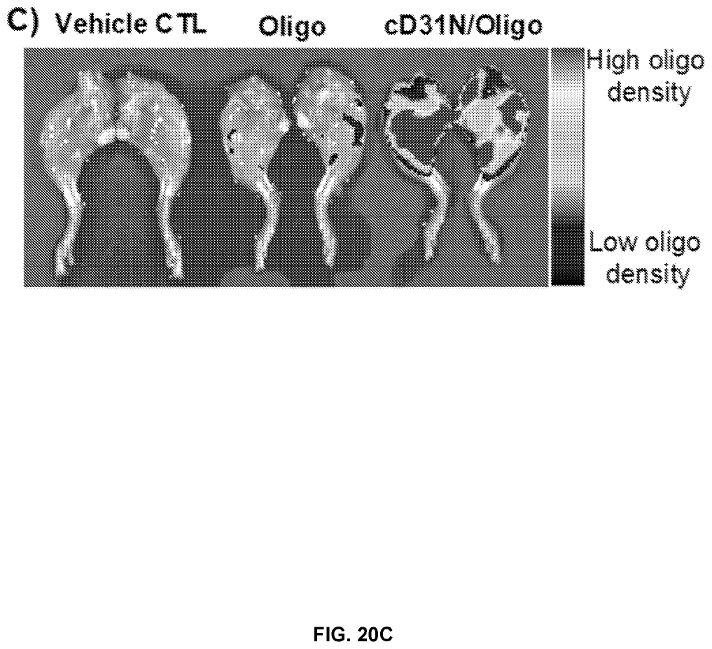

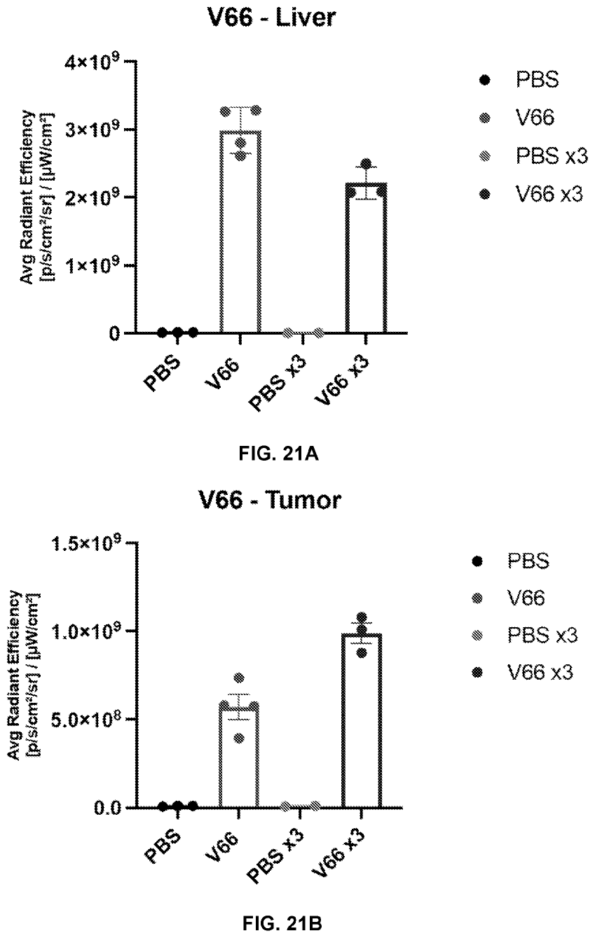

In some embodiments, a humanized 3E10 antibody or antigen-binding fragment thereof has a sequence with high sequence identity, e.g., at least 95% identity, at least 96% identity, at least 97% identity, at least 99% identity, at least 99.5% identity, or 100% identity with a humanized 3E10 variable light domains and/or humanized 3E10 variable heavy domains shown in FIG. 19 (heavy chain variable regions), FIG. 20 (heavy chain without signal sequence), FIG. 21 (heavy chain with signal peptide), FIG. 22 (light chain variable regions), FIG. 23 (light chain without signal sequence), and/or FIG. 24 (light chain with signal peptide). These variable light and variable heavy domains can be combined in any of the possible 42 combinations (each of the seven variable light domains with each of the variable heavy domains) to form humanized 3E10 antibodies and nucleic acid binding fragments (e.g., scFvs) thereof. Twenty-two (22) antibodies incorporating different combinations of these humanized VL and VH sequences were made, all of which bound nucleic acids. Further, when human leukemia cells were exposed to complexes formed between these antibodies and a RIG-I agonist polynucleotide, all of the complexes were able to generate a Type I IFN response, suggesting that all of the tested antibodies were able to deliver functional polynucleotides into the cells and affect a RIG-I mediated response (data not shown).

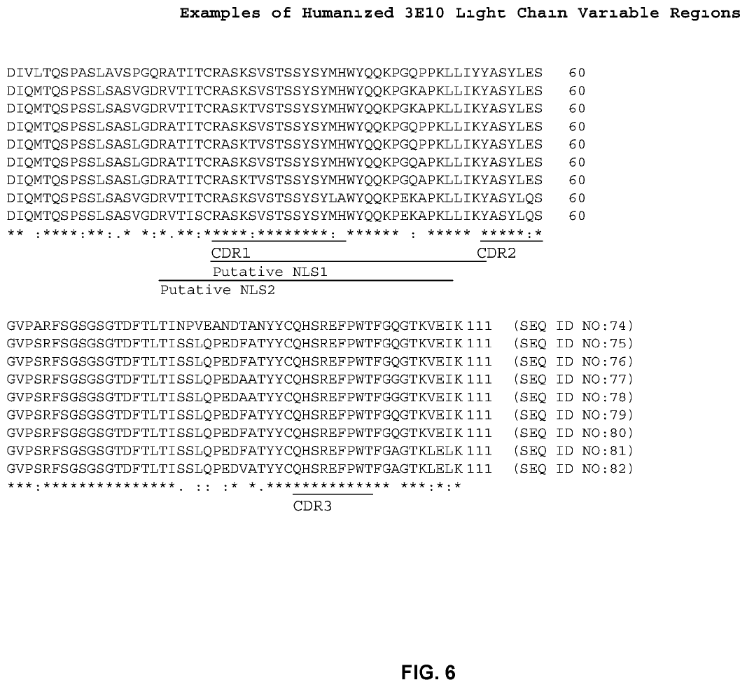

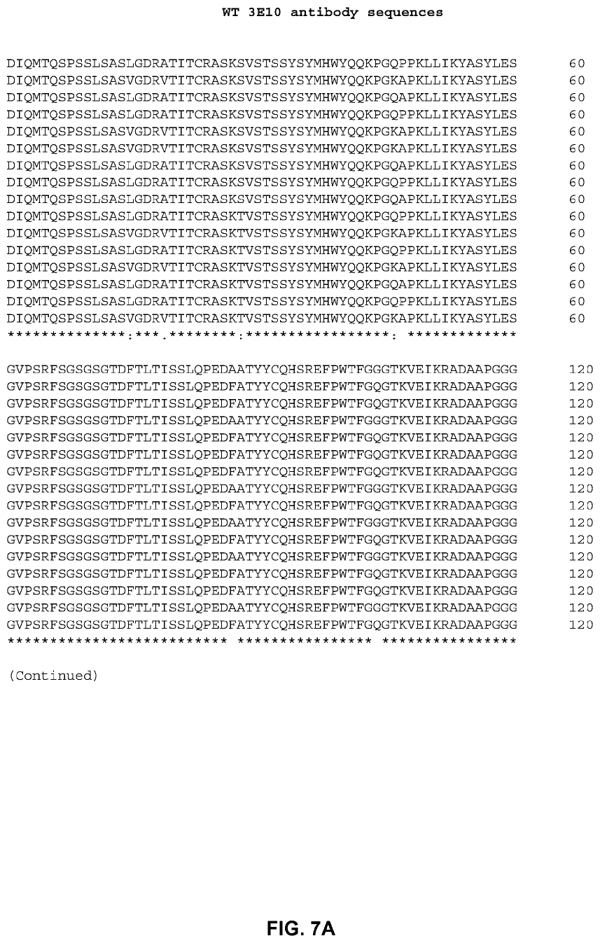

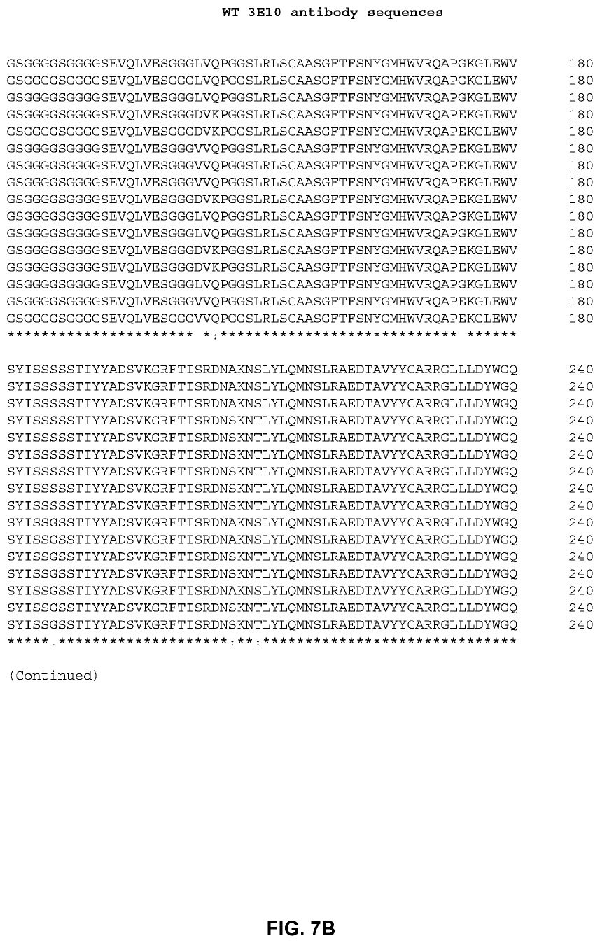

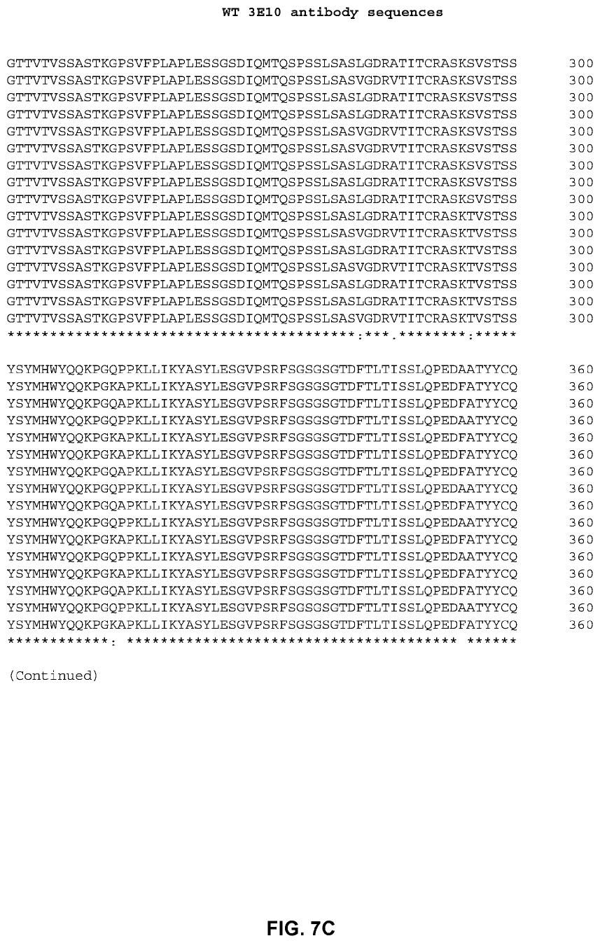

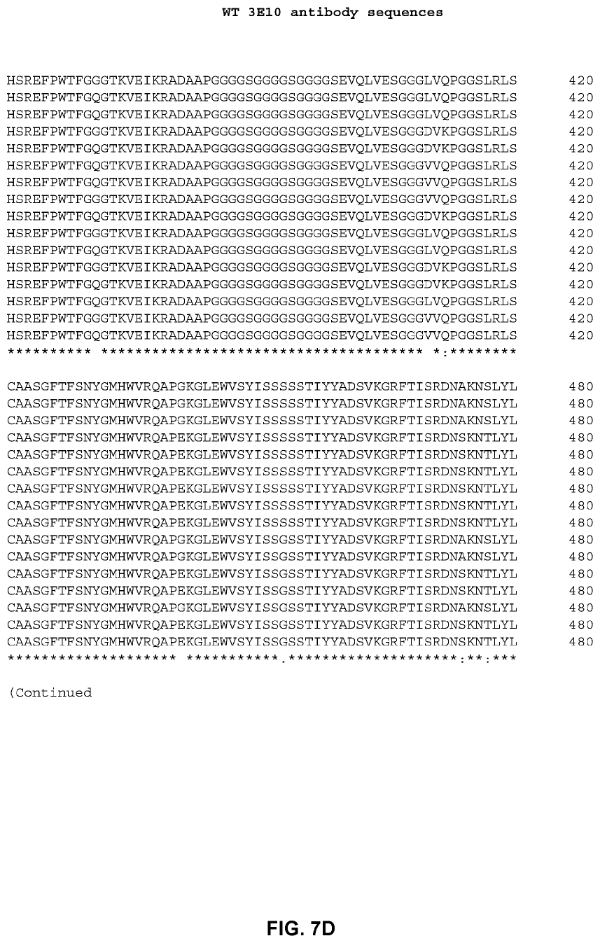

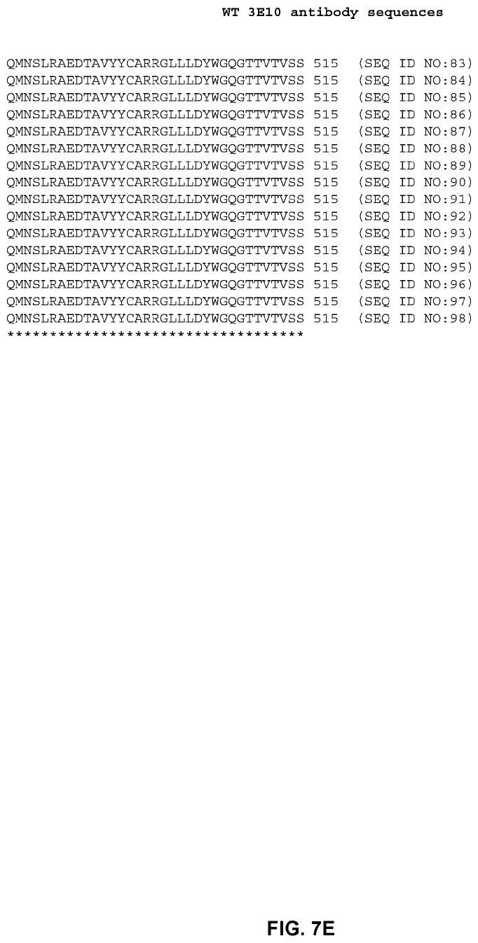

Accordingly, in some embodiments the disclosure provides humanized 3E10 antibodies and antigen-binding fragments thereof that incorporate any combination of the humanized VL and VH sequences shown in FIGS. 5-10, as well as VL and VH sequences having sequence identity thereto, e.g., having at least 90%, 91%, 92%, 93%, 94%, 95%, 96%, 97%, 98%, or 99% identity to a VH or VL sequence shown in FIGS. 19-24.

In some embodiments, a humanized 3E10 antibody or antigen binding fragment thereof, described herein includes a light chain variable domain (3E10-VL) comprising an amino acid sequence that is at least 95%, 96%, 97%, 98%, 99%, 99.5%, or 100% identical to an amino acid sequence selected from the group consisting of 3E10-VL-h1 (SEQ ID NO:120), 3E10-VL-h2 (SEQ ID NO:121), 3E10-VL-h3 (SEQ ID NO:122), 3E10-VL-h4 (SEQ ID NO:123), 3E10-VL-h5 (SEQ ID NO:124), and 3E10-VL-h6 (SEQ ID NO:125) and a heavy chain variable domain (3E10-VH) comprising an amino acid sequence that is at least 95%, 96%, 97%, 98%, 99%, 99.5%, or 100% identical to an amino acid sequence selected from the group consisting of 3E10-VH-h1 (SEQ ID NO:99), 3E10-VH-h2 (SEQ ID NO:100), 3E10-VH-h3 (SEQ ID NO: 101), 3E10-VH-h4 (SEQ ID NO:102), 3E10-VH-h5 (SEQ ID NO:103), 3E10-VH-h6 (SEQ ID NO: 104), and 3E10-VH-h7 (SEQ ID NO:105).

In some embodiments, the sequence of the 3E10-VL is at least 95% identical to 3E10-VL-h6 (SEQ ID NO:125). In some embodiments, the sequence of the 3E10-VL is at least 96% identical to 3E10-VL-h6 (SEQ ID NO:125). In some embodiments, the sequence of the 3E10-VL is at least 97% identical to 3E10-VL-h6 (SEQ ID NO:125). In some embodiments, the sequence of the 3E10-VL is at least 98% identical to 3E10-VL-h6 (SEQ ID NO:125). In some embodiments, the sequence of the 3E10-VL is at least 99% identical to 3E10-VL-h6 (SEQ ID NO: 125). In some embodiments, the sequence of the 3E10-VL is 3E10-VL-h6 (SEQ ID NO: 125).

In some embodiments, the sequence of the 3E10-VH is at least 95% identical to 3E10-VH-h6 (SEQ ID NO:104). In some embodiments, the sequence of the 3E10-VH is at least 96% identical to 3E10-VH-h6 (SEQ ID NO:104). In some embodiments, the sequence of the 3E10-VH is at least 97% identical to 3E10-VH-h6 (SEQ ID NO:104). In some embodiments, the sequence of the 3E10-VH is at least 98% identical to 3E10-VH-h6 (SEQ ID NO:104). In some embodiments, the sequence of the 3E10-VH is at least 99% identical to 3E10-VH-h6 (SEQ ID NO: 104). In some embodiments, the sequence of the 3E10-VH is 3E10-VH-h6 (SEQ ID NO: 104).

In some embodiments, a humanized 3E10 antibody or antigen binding fragment thereof, described herein includes a light chain (3E10-LC) comprising an amino acid sequence that is at least 95%, 96%, 97%, 98%, 99%, 99.5%, or 100% identical to an amino acid sequence selected from the group consisting of 3E10-LC-h1m (SEQ ID NO:126), 3E10-LC-h2m (SEQ ID NO: 127), 3E10-LC-h3m (SEQ ID NO:128), 3E10-LC-h4m (SEQ ID NO:129), 3E10-LC-h5m (SEQ ID NO:130), and 3E10-LC-h6m (SEQ ID NO:131) and a heavy chain (3E10-HC) comprising an amino acid sequence that is at least 95%, 96%, 97%, 98%, 99%, 99.5%, or 100% identical to an amino acid sequence selected from the group consisting of 3E10-HC-h1m (SEQ ID NO: 106), 3E10-HC-h2m (SEQ ID NO:107), 3E10-HC-h3m (SEQ ID NO:108), 3E10-HC-h4m (SEQ ID NO:109), 3E10-HC-h5m (SEQ ID NO:110), 3E10-HC-h6m (SEQ ID NO:111), and 3E10-HC-h7m (SEQ ID NO:112).

In some embodiments, the sequence of the 3E10-LC is at least 95% identical to 3E10-LC-h6m (SEQ ID NO:131). In some embodiments, the sequence of the 3E10-LC is at least 96% identical to 3E10-LC-h6m (SEQ ID NO:131). In some embodiments, the sequence of the 3E10-LC is at least 97% identical to 3E10-LC-h6m (SEQ ID NO:131). In some embodiments, the sequence of the 3E10-LC is at least 98% identical to 3E10-LC-h6m (SEQ ID NO: 131). In some embodiments, the sequence of the 3E10-LC is at least 99% identical to 3E10-LC-h6m (SEQ ID NO:131). In some embodiments, the sequence of the 3E10-LC is 3E10-LC-h6m (SEQ ID NO:131).

In some embodiments, the sequence of the 3E10-HC is at least 95% identical to 3E10-HC-h6m (SEQ ID NO:111). In some embodiments, the sequence of the 3E10-HC is at least 96% identical to 3E10-HC-h6m (SEQ ID NO:111). In some embodiments, the sequence of the 3E10-HC is at least 97% identical to 3E10-HC-h6m (SEQ ID NO:111). In some embodiments, the sequence of the 3E10-HC is at least 98% identical to 3E10-HC-h6m (SEQ ID NO: 111). In some embodiments, the sequence of the 3E10-HC is at least 99% identical to 3E10-HC-h6m (SEQ ID NO:111). In some embodiments, the sequence of the 3E10-HC is 3E10-HC-h6m (SEQ ID NO:111).

In some embodiments, a humanized 3E10 antibody or antigen binding fragment thereof described herein, includes a light chain (3E10-LC) comprising an amino acid sequence that is at least 95% identical to an amino acid sequence selected from the group consisting of 3E10-LC-h1 (SEQ ID NO:132), 3E10-LC-h2 (SEQ ID NO:133), 3E10-LC-h3 (SEQ ID NO: 134), 3E10-LC-h4 (SEQ ID NO:135), 3E10-LC-h5 (SEQ ID NO:136), and 3E10-LC-h6 (SEQ ID NO:137) and a heavy chain (3E10-HC) comprising an amino acid sequence that is at least 95% identical to an amino acid sequence selected from the group consisting of 3E10-HC-h1 (SEQ ID NO:113), 3E10-HC-h2 (SEQ ID NO:114), 3E10-HC-h3 (SEQ ID NO:115), 3E10-HC-h4 (SEQ ID NO:116), 3E10-HC-h5 (SEQ ID NO:117), 3E10-HC-h6 (SEQ ID NO:118), and 3E10-HC-h7 (SEQ ID NO:119).

In some embodiments, the sequence of the 3E10-LC is at least 95% identical to 3E10-LC-h6 (SEQ ID NO: 137). In some embodiments, the sequence of the 3E10-LC is at least 96% identical to 3E10-LC-h6 (SEQ ID NO:137). In some embodiments, the sequence of the 3E10-LC is at least 97% identical to 3E10-LC-h6 (SEQ ID NO:137). In some embodiments, the sequence of the 3E10-LC is at least 98% identical to 3E10-LC-h6 (SEQ ID NO:137). In some embodiments, the sequence of the 3E10-LC is at least 99% identical to 3E10-LC-h6 (SEQ ID NO: 137). In some embodiments, the sequence of the 3E10-LC is 3E10-LC-h6 (SEQ ID NO: 137).

In some embodiments, the sequence of the 3E10-HC is at least 95% identical to 3E10-HC-h6 (SEQ ID NO:118). In some embodiments, the sequence of the 3E10-HC is at least 96% identical to 3E10-HC-h6 (SEQ ID NO:118). In some embodiments, the sequence of the 3E10-HC is at least 97% identical to 3E10-HC-h6 (SEQ ID NO:118). In some embodiments, the sequence of the 3E10-HC is at least 98% identical to 3E10-HC-h6 (SEQ ID NO:118). In some embodiments, the sequence of the 3E10-HC is at least 99% identical to 3E10-HC-h6 (SEQ ID NO: 118). In some embodiments, the sequence of the 3E10-HC is 3E10-HC-h6 (SEQ ID NO: 118).

In some embodiments, a humanized 3E10 antibody or antigen binding fragment thereof described herein comprises a combination of a heavy chain variable domain (VH) and a light chain variable domain (VL) comprising amino acid sequences having at least 95%, 96%, 97%, 98%, 99%, 99.5%, or 100% sequence identity to a pair of VL and VH selected from 3E10-VH-h1 and 3E10-VL-h1, 3E10-VH-h1 and 3E10-VL-h2, 3E10-VH-h1 and 3E10-VL-h3, 3E10-VH-h1 and 3E10-VL-h4, 3E10-VH-h2 and 3E10-VL-h1, 3E10-VH-h2 and 3E10-VL-h2, 3E10-VH-h2 and 3E10-VL-h3, 3E10-VH-h2 and 3E10-VL-h4, 3E10-VH-h3 and 3E10-VL-h1, 3E10-VH-h3 and 3E10-VL-h2, 3E10-VH-h3 and 3E10-VL-h3, 3E10-VH-h3 and 3E10-VL-h4, 3E10-VH-h4 and 3E10-VL-h1, 3E10-VH-h4 and 3E10-VL-h2, 3E10-VH-h4 and 3E10-VL-h3, 3E10-VH-h4 and 3E10-VL-h4, 3E10-VH-h5 and 3E10-VL-h5, 3E10-VH-h5 and 3E10-VL-h6, 3E10-VH-h6 and 3E10-VL-h5, 3E10-VH-h6 and 3E10-VL-h6, 3E10-VH-h7 and 3E10-VL-h5, and 3E10-VH-h7 and 3E10-VL-h6.

In some embodiments, a humanized 3E10 antibody or antigen binding fragment thereof described herein comprises a heavy chain variable domain (VH) comprising an amino acid sequence having at least 95%, 96%, 97%, 98%, 99%, 99.5%, or 100% sequence identity to 3E10-VH-h6 (SEQ ID NO:104) and a light chain variable domain (VL) comprising an amino acid sequence having at least 95%, 96%, 97%, 98%, 99%, 99.5%, or 100% sequence identity to 3E10-VL-h6 (SEQ ID NO:125).

In some embodiments, a humanized 3E10 antibody or antigen binding fragment thereof described herein includes a light chain variable domain (3E10-VL) comprising an amino acid sequence that is at least 90%, 91%, 92%, 93%, 94%, 95%, 96%, 97%, 98%, 99%, 99.5%, or 100% identical to an amino acid sequence selected from the group consisting of 3E10-VL-h1 (SEQ ID NO:120), 3E10-VL-h2 (SEQ ID NO: 121), 3E10-VL-h3 (SEQ ID NO:122), 3E10-VL-h4 (SEQ ID NO:123), 3E10-VL-h5 (SEQ ID NO:124), and 3E10-VL-h6 (SEQ ID NO:125), where the light chain variable domain (3E10-VL) comprises one or more amino acid residues selected from proline (Pro) at position 15, threonine (Thr) at position 22, tyrosine (Tyr) at position 49, Thr at position 74, asparaginc (Asn) at position 76, alanine (Ala) at position 80, Asn at position 81, Thr at position 83, Asn at position 85, and valine (Val) at position 104, of the 3E10-VL according to Kabat numbering, and a set of 3E10-VL CDRs collectively having no more than 6 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11), and where the antibody includes a set of 3E10-VL CDRs collectively having no more than 6 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11).

In some embodiments, the humanized 3E10 antibody or antigen binding fragment thereof includes a set of 3E10-VL CDRs comprising no more than 5 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11). In some embodiments, the humanized 3E10 antibody or antigen binding fragment thereof includes a set of 3E10-VL CDRs comprising no more than 4 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11). In some embodiments, the humanized 3E10 antibody or antigen binding fragment thereof includes a set of 3E10-VL CDRs comprising no more than 3 amino acid substitutions relative to the set of CDRs having the amino acid sequences of 3E10-VL-CDR1 (SEQ ID NO:9), 3E10-VL-CDR2 (SEQ ID NO: 10), 3E10-VL-CDR3 (SEQ ID NO: 11). In some embodiments, the humanized 3E10 antibody or antigen binding fragment thereof includes a set of 3E10-VL CDRs comprising no more than 2 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11). In some embodiments, the humanized 3E10 antibody or antigen binding fragment thereof includes a set of 3E10-VL CDRs comprising no more than 1 amino acid substitution relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11). In some embodiments, the humanized 3E10 antibody or antigen binding fragment thereof includes a set of 3E10-VL CDRs having the amino acid sequences of 3E10-VL-CDR1 (SEQ ID NO:9), 3E10-VL-CDR2 (SEQ ID NO:10), 3E10-VL-CDR3 (SEQ ID NO:11).

In some embodiments, the humanized 3E10 antibody or antigen binding fragment thereof has a lysine (Lys) residue at position 49 of the 3E10-VL according to Kabat numbering. In some embodiments, the humanized 3E10 antibody or antigen binding fragment thereof has a glutamic acid (Glu) residue at position 81 of the 3E10-VL according to Kabat numbering. In some embodiments, the humanized 3E10 antibody or antigen binding fragment thereof has a proline (Pro) residue at position 15 of the 3E10-VL according to Kabat numbering. In some embodiments, the humanized 3E10 antibody or antigen binding fragment thereof has a valine (Val) residue at position 104 of the 3E10-VL according to Kabat numbering.

In some embodiments, a humanized 3F10 antibody or antigen binding fragment thereof described herein includes a heavy chain variable domain (3E10-VH) comprising an amino acid sequence that is at least 90%, 91%, 92%, 93%, 94%, 95%, 96%, 97%, 98%, 99%, 99.5%, or 100% identical to an amino acid sequence selected from the group consisting of 3E10-VH-h1 (SEQ ID NO:99), 3E10-VH-h2 (SEQ ID NO:100), 3E10-VH-h3 (SEQ ID NO:101), 3E10-VH-h4 (SEQ ID NO:102), 3E10-VH-h5 (SEQ ID NO:103), 3E10-VH-h6 (SEQ ID NO: 104), and 3E10-VH-h7 (SEQ ID NO:105), where the heavy chain variable domain (3E10-VH) comprises one or more amino acid residues selected from glutamine (Gln) at position 13, leucine (Leu) at position 18, arginine (Arg) at position 19, glycine (Gly) at position 42, serine (Ser) at position 49, Ser at position 77, tyrosine (Tyr) at position 79, Asn at position 82, Ala at position 84, Val at position 89, leucine (Leu) at position 108, Val at position 109, and Ser at position 113, of the 3E10-VH according to Kabat numbering, and where the antibody includes a set of 3E10-VH CDRs collectively having no more than 6 amino acid substitutions relative to the set of CDRs having the amino acid sequences of 3E10-VH-CDR1_D3IN (SEQ ID NO:15), 3E10-VH-CDR2 (SEQ ID NO:4), and 3E10-VH-CDR3 (SEQ ID NO:5).

In some embodiments, the humanized 3E10 antibody or antigen binding fragment thereof includes a set of 3E10-VH CDRs comprising no more than 5 amino acid substitutions relative to the set of CDRs having the amino acid sequences of 3E10-VH-CDR1_D3IN (SEQ ID NO: 15), 3E10-VH-CDR2 (SEQ ID NO:4), and 3E10-VH-CDR3 (SEQ ID NO:5). In some embodiments, the humanized 3E10 antibody or antigen binding fragment thereof includes a set of 3E10-VH CDRs comprising no more than 4 amino acid substitutions relative to the set of CDRs having the amino acid sequences of 3E10-VH-CDR1_D3IN (SEQ ID NO:15), 3E10-VH-CDR2 (SEQ ID NO:4), and 3E10-VH-CDR3 (SEQ ID NO:5). In some embodiments, the humanized 3E10 antibody or antigen binding fragment thereof includes a set of 3E10-VH CDRs comprising no more than 3 amino acid substitutions relative to the set of CDRs having the amino acid sequences of 3E10-VH-CDR1_D3IN (SEQ ID NO:15), 3E10-VH-CDR2 (SEQ ID NO:4), and 3E10-VH-CDR3 (SEQ ID NO:5). In some embodiments, the humanized 3E10 antibody or antigen binding fragment thereof includes set of 3E10-VH CDRs comprising no more than 2 amino acid substitutions relative to the set of CDRs having the amino acid sequences of 3E10-VH-CDR1_D31N (SEQ ID NO:15), 3E10-VH-CDR2 (SEQ ID NO:4), and 3E10-VH-CDR3 (SEQ ID NO:5). In some embodiments, the humanized 3E10 antibody or antigen binding fragment thereof includes a set of 3E10-VH CDRs comprising no more than 1 amino acid substitution relative to the set of CDRs having the amino acid sequences of 3E10-VH-CDR1_D3IN (SEQ ID NO:15), 3E10-VH-CDR2 (SEQ ID NO:4), and 3E10-VH-CDR3 (SEQ ID NO: 5). In some embodiments, the humanized 3E10 antibody or antigen binding fragment thereof includes a set of 3E10-VH CDRs having the amino acid sequences of 3E10-VH-CDR1_D3IN (SEQ ID NO:15), 3E10-VH-CDR2 (SEQ ID NO:4), and 3E10-VH-CDR3 (SEQ ID NO: 5).

In some embodiments, the humanized 3E10 antibody or antigen binding fragment thereof has an arginine (Arg) residue at position 18 of the 3E10-VH according to Kabat numbering. In some embodiments, the humanized 3E10 antibody or antigen binding fragment thereof has a (Lys) residue at position 19 of the 3E10-VH according to Kabat numbering. In some embodiments, the humanized 3E10 antibody or antigen binding fragment thereof has an alanine (Ala) residue at position 49 of the 3E10-VH according to Kabat numbering. In some embodiments, the humanized 3E10 antibody or antigen binding fragment thereof with a glutamine (Gln) residue at position 13 of the 3E10-VH according to Kabat numbering. In some embodiments, the humanized 3E10 antibody or antigen binding fragment thereof has a leucine (Leu) residue at position 108 of the 3E10-VH according to the Kabat numbering. In some embodiments, the humanized 3E10 antibody or antigen binding fragment thereof with a Val residue at position 109 of the 3E10-VH according to Kabat numbering. In some embodiments, the humanized 3E10 antibody or antigen binding fragment thereof has a serine (Ser) residue at position 113 of the 3E10-VH according to Kabat numbering.

### Cell Penetration and Nuclear Localization

The disclosed compositions and methods typically utilize antibodies that maintain the ability to penetrate cells, and optionally nuclei.

The mechanisms of cellular internalization by autoantibodies are diverse. Some are taken into cells through electrostatic interactions or FcR-mediated endocytosis, while others utilize mechanisms based on association with cell surface myosin or calreticulin, followed by endocytosis (Ying-Chyi et al., Eur. J. Immunol. 38, 3178-3190 (2008), Yanase et al., J. Clin. Invest. 100, 25-31 (1997)). 3E10 penetrates cells in an Fc-independent mechanism (as evidenced by the ability of 3E10 fragments lacking an Fc to penetrate cells) but involves presence of the nucleoside transporter ENT2 (Weisbart et al., Scientific Reports volume 5, Article number: 12022 (2015), Zack et al., J. Immunol. 157, 2082-2088 (1996), Hansen et al., J. Biol. Chem. 282, 20790-20793 (2007)). Thus, in some embodiments, the antibodies utilized in the disclosed compositions and methods are ones that penetrates cells in an Fc-independent mechanism but involves presence of the nucleoside transporter ENT2.

Mutations in 3E10 that interfere with its ability to bind DNA may render the antibody incapable of nuclear penetration. Thus, typically the disclosed variants and humanized forms of the antibody maintain the ability to bind nucleic acids, particularly DNA. In addition, 3E10 scFv has previously been shown capable of penetrating into living cells and nucleic in an ENT2-dependent manner, with efficiency of uptake impaired in ENT2-deficient cells (Hansen, et al., J. Biol. Chem. 282, 20790-20793 (2007)). Thus, in some embodiments, the disclosed variants and humanized forms of the antibody maintain the ability to penetrate into cell nuclei in an ENT-dependent, preferably ENT2-dependent manner.

As discussed in US 2021/0054102 and US 2021/0137960, some humanized 3E10 variant were found to penetrate cell nuclei more efficiently than the original murine 3E10 (D31N) di-scFv, while others were found to have lost the ability to penetrate nuclei. In particular, variants 10 and 13 penetrated nuclei very well compared to the murine antibody.

Potential bipartite nuclear localization signals (NLS) in humanized 3E10 VL have been identified and may include part or all of the following sequences:

(SEQ ID NO: 143)
RASKSVSTSSYSYMHWYQQKPGOPPKLLIKY;
(SEQ ID NO: 144)
RASKTVSTSSYSYMHWYQQKPGQPPKLLIKY;
or
(SEQ ID NO: 145)
RVTITCRASKSVSTSSYSYMHWYQQKPGKAPKL.

An example consensus NLS can be, or include, (X)RASKTVSTSSYSYMHWYQQKPGQPPKLL(X)KY (where (X)=any residue, but preferentially is a basic residue (R or K) (SEQ ID NO:146) or a variant thereof with at least 60, 65, 70, 75, 80, 85, 90, 95, 96, 97, 98, 99 percent sequence identity to SEQ ID NO:147.

Thus, in some embodiments, particularly where nuclear importation is important, the disclosed antibodies may include the sequence of any one of SEQ ID NOs: 143-147, or fragments and variants thereof (e.g., at least 70, 75, 80, 85, 90, 95, 96, 97, 98, 99, or 100% amino acid sequence identity with any one of SEQ ID NOs: 143-147) that can translocate into the nucleus of a cell.

Presence of an NLS indicates that a humanized 3E10 antibody or antigen binding fragment thereof may cross the nuclear envelope via the nuclear import pathway. In some embodiments, the NLS improves importation by interacting with one or more members of the import pathway. Thus, in some embodiments, the NLS can bind to importin-β, an importin-β/importin-α heterodimer, or a combination thereof.

### Nucleic Acid Binding

The disclosed compositions and methods typically utilize antibodies that maintain the ability to bind mRNA.

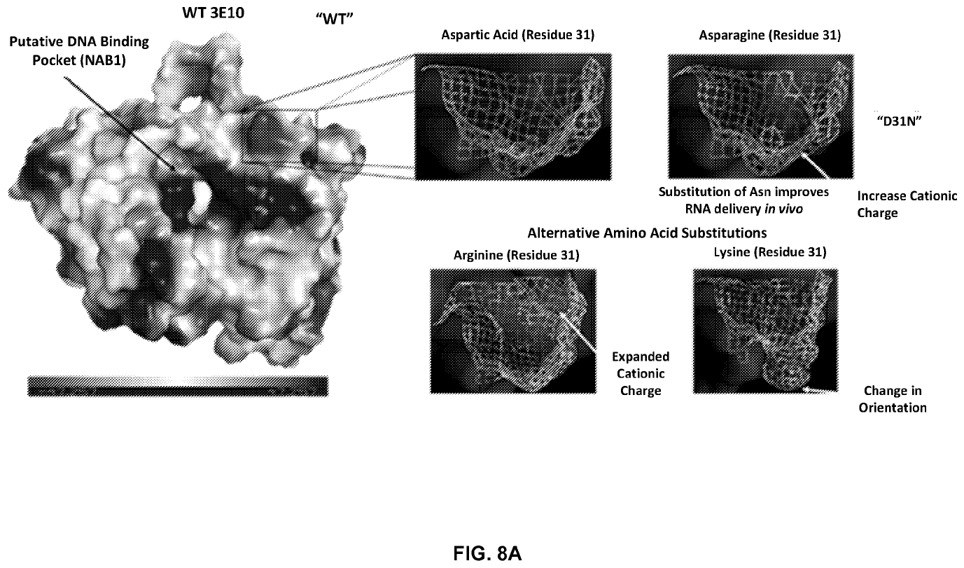

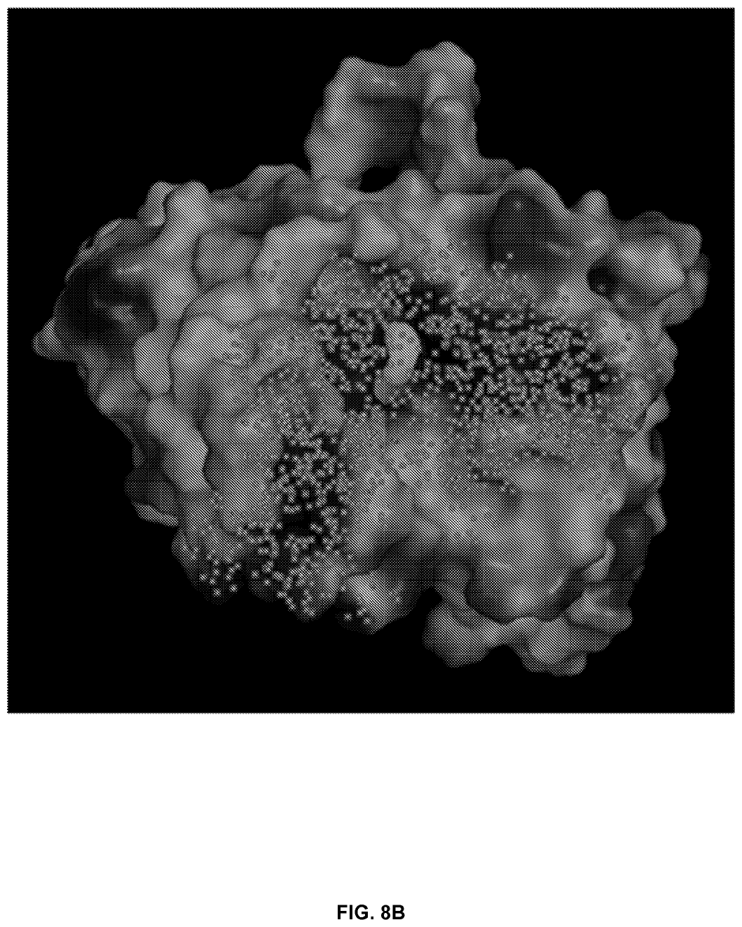

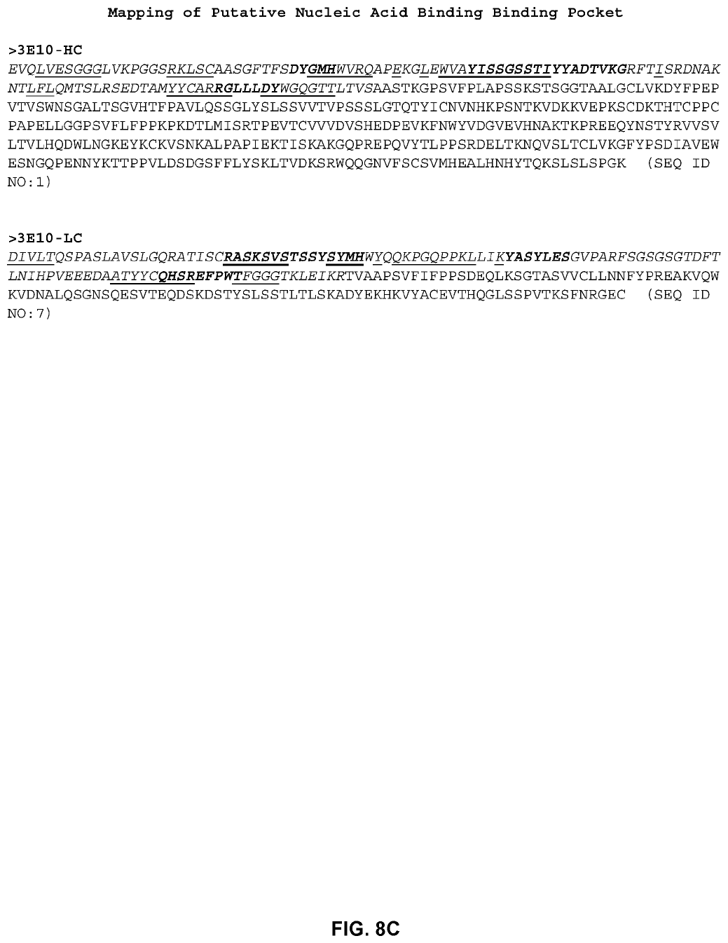

Example 2 described molecular modeling of 3E10 and additional 3E10 variants. Molecular modeling of 3E10 (Pymol) revealed a putative Nucleic Acid Binding pocket (NAB1) (see, e.g., FIGS. 8A and 8B), and illustrated with underlining in FIG. 8C.

In some embodiments, the disclosed antibodies include some or all of the underlined NAB1 sequences. In some embodiments, the antibodies include a variant sequence that has an altered ability of bind nucleic acids. In some embodiments, the mutations (e.g., substitutions, insertions, and/or deletions) in the NAB1 improve binding of the antibody to nucleic acids such as RNA. In some embodiments, the mutations are conservative substitutions. In some embodiments, the mutations increase the cationic charge of the NAB1 pocket.

As discussed and exemplified herein, mutation of aspartic acid at residue 31 of CDR1 to asparagine increased the cationic charge of this residue and enhanced nucleic acid binding and delivery in vivo (3E10-D31N). Additional exemplary variants include mutation of aspartic acid at residue 31 of CDR1 to arginine (3E10-D31R), which modeling indicates expands cationic charge, or lysine (3E10-D31K) which modeling indicates changes charge orientation. Thus, in some embodiments, the 3E10 binding protein includes a D31R or D31K substitution.

All of the sequences disclosed herein having the residue corresponding to 3E10 D31 or N31, are expressly disclosed with a D31R or D31K or N31R or N31K substitution therein.

Molecular modeling of 3E10 (Pymol) revealed a putative Nucleic Acid Binding pocket (NAB1) (FIGS. 8A-8B). Mutation of aspartic acid at residue 31 of CDR1 to asparagine increased the cationic charge of this residue and enhanced nucleic acid binding and delivery in vivo (3E10-D31N). Mutation of aspartic acid at residue 31 of CDR1 to arginine (3E10-D31R), further expanded the cationic charge while mutation to lysine (3E10-D31K) changed charge orientation (FIG. 8A).

NAB1 amino acids predicted from molecular modeling have been underlined in the heavy and light chain sequences in FIG. 8C. FIG. 8B is an illustration showing molecular modeling of 3E10-scFv (Pymol) with NAB1 amino acid residues illustrated with punctate dots.

Gene replacement therapy refers to a number of therapeutic techniques for delivering a functional copy of a gene to a tissue in need of the protein encoded by the gene, including DNA-based gene therapy techniques in which a functional copy of the gene is transcribed within the cell, e.g., with or without being stably integrated into the genome of the subject, gene editing therapies, such as CRISPR/Cas, that repair or replace mutant copies of the gene or specific nucleotides in the host's genome, and mRNA delivery-based approaches in which mRNA encoding the protein are delivered to the cell, eliminating the need to transcribe an exogenous copy of the gene. Researchers have developed, and continue to develop, gene replacement therapies for a diverse set of disorders, most notably genetic disorders and cancers in a subject has one or two mutant or non-functioning copies of the gene, e.g., due to mutations in the gene that cause partial or complete loss-of-function, mutations in an associated regulatory region that down-regulates gene transcription, and/or small genomic deletions.

### Hepatic Diseases

In some embodiments, the present disclosure provides methods for treating a hepatic disease in a subject by delivering a complex of a therapeutic mRNA encoding a protein expressed in the liver and a 3E10 antibody or antigen binding fragment thereof, as described herein, to liver tissue of the subject.

Although, in some embodiments, the polypeptide encoded by the mRNA is a wild-type version of the protein expressed in the liver, it will be appreciated that naturally occurring variants or synthetically engineered versions of the protein may also find use in the compositions and methods described herein. For example, in instances where mRNA therapy is used for enzyme replacement therapy, it is common for the enzyme encoded by the mRNA to be engineered to improve enzymatic activity. Further, in certain instances, where the wild type version of a therapeutic protein is particularly large and/or includes one or more domains that are particularly susceptible to proteolytic degradation, is it common for the protein encoded by a gene therapy vector to be engineered to make the protein smaller and/or to remove susceptible regions that are dispensable for protein function.

In some embodiments, the therapeutic mRNA molecule encodes for a protein expressed in the liver that is associated with a disease or disorder. For example, in some embodiments, the disease or disorder is associated with a mutation in a gene expressed in the liver. See, for example, Scorza M et al., “Genetic diseases that predispose to early liver cirrhosis,” Int J Hepatol., 2014:713754 (2014), the disclosure of which is incorporated herein by reference, in its entirety, for all purposes. Examples of the genes expressed in the liver that are associated with disease are listed in Table 2, below. Accordingly, in some embodiments, a subject with a particular disease is treated by administration of a 3E10-mRNA complex where the mRNA encodes for a polypeptide corresponding to an associated gene in Table 2. For example, in one embodiment, a mRNA molecule encoding a polypeptide associated with the Copper-transporting ATPase 2 (ATP7B) protein is used for the treatment of Wilson's disease.

TABLE 2
Example genes expressed in the liver that are mutated in various diseases.

| Disease | Mutant Protein (Genes) |
|---|---|
| Wilson disease | Copper-transporting ATPase 2 (ATP7B = |
| | NG_008806.1); Homo sapiens ATPase copper |
| | transporting beta (ATP7B), RefSeqGene on |
| | chromosome 13 |
| Progressive familial intrahepatic | Phosphatidylcholine translocator ABCB4 (ABCB4 = |
| cholestasis type 3 | NG_007118.2); Homo sapiens ATP binding cassette |
| | subfamily B member 4 (ABCB4), RefSeqGene on |
| | chromosome 7 |
| Hereditary fructose intolerance | Fructose-bisphosphate aldolase B (ALDOB = |
| | NG_012387.1); Homo sapiens aldolase, fructose- |
| | bisphosphate B (ALDOB), RefSeqGene (LRG_1244) on |
| | chromosome 9 |
| Glycogen storage disease type IV | 1,4-alpha-glucan-branching enzyme (GBE1 = |
| | NG_011810.1); Homo sapiens 1,4-alpha-glucan |
| | branching enzyme 1 (GBE1), RefSeqGene on |
| | chromosome 3 |
| Tyrosinemia type I | Fumarylacetoacetase (FAH = NG_012833.1); Homo |
| | sapiens fumarylacetoacetate hydrolase (FAH), |
| | RefSeqGene on chromosome 15 |
| Argininosuccinate lyase | Argininosuccinate lyase (ASL = NG_009288.1); Homo |
| deficiency | sapiens argininosuccinate lyase (ASL), RefSeqGene on |
| | chromosome 7 |
| Citrin deficiency | Calcium-binding mitochondrial carrier protein |
| Citrullinemia 2 (CTLN2) | Aralar2 (SLC25A13 = NG_012247.2); Homo sapiens |
| Cholestasis, neonatal intrahepatic, | solute carrier family 25 member 13 (SLC25A13), |
| caused by citrin deficiency | RefSeqGene on chromosome 7; nuclear gene for |
| (NICCD) | mitochondrial product |
| Cholesteryl ester storage disease | Lysosomal acid lipase/cholesteryl ester hydrolase |
| | (LIPA = NG_008194.1); Homo sapiens lipase A, |
| | lysosomal acid type (LIPA), RefSeqGene on |
| | chromosome 10 |
| Alpha-1 antitrypsin deficiency | Alpha-1-antitrypsin (SERPINA1 = NG_008290.1); |
| | Homo sapiens serpin family A member 1 (SERPINA1), |
| | RefSeqGene (LRG_575) on chromosome 14 |
| Cystic fibrosis | Cystic fibrosis transmembrane conductance regulator |
| | (CFTR = NG_016465.4); Homo sapiens CF |
| | transmembrane conductance regulator (CFTR), |
| | RefSeqGene (LRG_663) on chromosome 7 |
| Hereditary hemochromatosis | Hereditary hemochromatosis protein (HFE = |
| | NG_008720.2); Homo sapiens homeostatic iron regulator |
| | (HFE), RefSeqGene (LRG_748) on chromosome 6 |
| Alström syndrome | Alstrom syndrome protein 1 (ALMS1 = NG_011690.1); |
| | Homo sapiens ALMS1 centrosome and basal body |
| | associated protein (ALMS1), RefSeqGene (LRG_741) on |
| | chromosome 2 |
| Hemophilia A | Coagulation Factor VIII (F8 = NG_011403.2); Homo |
| | sapiens coagulation factor VIII (F8), RefSeqGene |
| | (LRG_555) on chromosome X |
| Hemophilia B | Coagulation Factor IX (F9 = NG_007994.1); Homo |
| | sapiens coagulation factor IX (F9), RefSeqGene |
| | (LRG_556) on chromosome X |

### Cardiac Muscle Diseases

In some embodiments, the present disclosure provides methods for treating a cardiac muscle disease in a subject by delivering a complex of a therapeutic mRNA encoding a protein expressed in the cardiac muscle and a 3E10 antibody or antigen binding fragment thereof, as described herein, to cardiac muscle tissue of the subject.

Although, in some embodiments, the polypeptide encoded by the mRNA is a wild-type version of the protein expressed in cardiac muscle, it will be appreciated that naturally occurring variants or synthetically engineered versions of the protein may also find use in the compositions and methods described herein. For example, in instances where mRNA therapy is used for enzyme replacement therapy, it is common for the enzyme encoded by the mRNA to be engineered to improve enzymatic activity. Further, in certain instances, where the wild type version of a therapeutic protein is particularly large and/or includes one or more domains that are particularly susceptible to proteolytic degradation, is it common for the protein encoded by a gene therapy vector to be engineered to make the protein smaller and/or to remove susceptible regions that are dispensable for protein function.

In some embodiments, the therapeutic mRNA molecule encodes for a protein expressed in cardiac muscle that is associated with a disease or disorder. For example, in some embodiments, the disease or disorder is associated with a mutation in a gene expressed in cardiac muscle. See, for example, Pugh T J et al., “The landscape of genetic variation in dilated cardiomyopathy as surveyed by clinical DNA sequencing,” Genet Med., 16 (8): 601-08 (2014), the disclosure of which is incorporated herein by reference, in its entirety, for all purposes.

Cardiomyopathy is a disease in which heart muscle is typically weakened or distorted, and functionally impaired. This can cause symptoms such as chest pain, breathlessness or palpitations, and frequently leads to heart failure. While cardiomyopathy symptoms can often be controlled by medication, devices such as pacemakers, or surgery, there is no cure for heart failure, and half of heart failure patients die within five years of diagnosis.

Examples of the genes expressed in cardiac muscle that are associated with disease are listed in Table 3, below. Accordingly, in some embodiments, a subject with a particular disease is treated by administration of a 3E10-mRNA complex where the mRNA encodes for a polypeptide corresponding to an associated gene in Table 3. For example, in one embodiment, a mRNA molecule encoding a polypeptide associated with the Myosin-binding protein C, cardiac-type (MYBPC3) protein is used for the treatment of Hypertrophic cardiomyopathy.

TABLE 3
Example genes expressed in cardiac muscle that are mutated in various diseases.

| Disease | Mutant Protein (Genes) |
|---|---|
| Hypertrophic cardiomyopathy | Myosin-binding protein C, cardiac-type (MYBPC3 = |
| (HCM) | NG_007667.1); Homo sapiens myosin binding protein C3 |
| | (MYBPC3), RefSeqGene (LRG_386) on chromosome 11 |
| Atrial fibrillation (AF) | Paired mesoderm homeobox protein 1 (PRRX1 = |
| | NG_031856.2); Homo sapiens paired related homeobox 1 |
| | (PRRX1), RefSeqGene on chromosome 1 |
| Atrial fibrillation (AF) | Potassium voltage-gated channel subfamily A member |
| | 5 (KCNA5 = NG_012198.1); Homo sapiens potassium |
| | voltage-gated channel subfamily A member 5 (KCNA5), |
| | RefSeqGene on chromosome 12 |
| Dilated cardiomyopathy (DCM) | BAG family molecular chaperone regulator 5 (BAG5 = |
| | NM_001015049.5); Homo sapiens BAG cochaperone 5 |
| | (BAG5), transcript variant 1, mRNA |
| | Titin (TTN = NG_011618.3); Homo sapiens titin (TTN), |
| | RefSeqGene (LRG_391) on chromosome 2 |
| | Nebulin-related-anchoring protein (NRAP = |
| | NM_001261463.2); Homo sapiens nebulin related |
| | anchoring protein (NRAP), transcript variant 3, mRNA |
| | Prelamin-A/C (LMNA = NG_008692.2); Homo sapiens |
| | lamin A/C (LMNA), RefSeqGene (LRG_254) on |
| | chromosome 1 |
| | Myosin-7 (MYH7 = NG_007884.1); Homo sapiens |
| | myosin heavy chain 7 (MYH7), RefSeqGene (LRG_384) |
| | on chromosome 14 |
| | Desmoplakin (DSP = NG_008803.1); Homo sapiens |
| | desmoplakin (DSP), RefSeqGene (LRG_423) on |
| | chromosome 6 |
| | Troponin T (TNNT2 = NG_007556.1); Homo sapiens |
| | troponin T2, cardiac type (TNNT2), RefSeqGene |
| | (LRG_431) on chromosome 1 |
| | Tropomyosin alpha-1 chain (TPM1 = NG_007557.1); |
| | Homo sapiens tropomyosin 1 (TPM1), RefSeqGene |
| | (LRG_387) on chromosome 15 |
| | RNA-binding protein 20 (RBM20 = NG_021177.1); |
| | Homo sapiens RNA binding motif protein 20 (RBM20), |
| | RefSeqGene (LRG_382) on chromosome 10 |
| | Vinculin (VCL = NG_008868.1); Homo sapiens vinculin |
| | (VCL), RefSeqGene (LRG_383) on chromosome 10 |
| | Desmin (DES = NG_008043.1); Homo sapiens desmin |
| | (DES), RefSeqGene (LRG_380) on chromosome 2 |
| Arrhythmogenic right ventricular | Desmoplakin (DSP = NG_008803.1); Homo sapiens |
| dysplasia, familial, 8 (ARVD8) | desmoplakin (DSP), RefSeqGene (LRG_423) on |
| | chromosome 6 |
| Cardiomyopathy, familial | Troponin T (TNNT2 = NG_007556.1); Homo sapiens |
| hypertrophic 2 (CMH2) | troponin T2, cardiac type (TNNT2), RefSeqGene |
| | (LRG_431) on chromosome 1 |
| Cardiomyopathy, dilated 1D | Troponin T (TNNT2 = NG_007556.1); Homo sapiens |
| (CMD1D) | troponin T2, cardiac type (TNNT2), RefSeqGene |
| | (LRG_431) on chromosome 1 |
| Cardiomyopathy, familial | Troponin T (TNNT2 = NG_007556.1); Homo sapiens |
| restrictive 3 (RCM3) | troponin T2, cardiac type (TNNT2), RefSeqGene |
| | (LRG_431) on chromosome 1 |
| Cardiomyopathy, familial | Tropomyosin alpha-1 chain (TPM1 = NG_007557.1); |
| hypertrophic 3 (CMH3) | Homo sapiens tropomyosin 1 (TPM1), RefSeqGene |
| | (LRG_387) on chromosome 15 |
| Left ventricular non-compaction | Tropomyosin alpha-1 chain (TPM1 = NG_007557.1); |
| 9 (LVNC9) | Homo sapiens tropomyosin 1 (TPM1), RefSeqGene |
| | (LRG_387) on chromosome 15 |
| Cardiomyopathy, familial | Vinculin (VCL = NG_008868.1); Homo sapiens vinculin |
| hypertrophic 15 (CMH15) | (VCL), RefSeqGene (LRG_383) on chromosome 10 |
| Myopathy, myofibrillar, 1 | Desmin (DES = NG_008043.1); Homo sapiens desmin |
| (MFM1) | (DES), RefSeqGene (LRG_380) on chromosome 2 |
| Duchenne and Becker | Dystrophin (DMD = NG_012232.1; Homo sapiens |
| muscular dystrophy | dystrophin (DMD), RefSeqGene (LRG_199) on |
| | chromosome X) |
| Autosomal-dominant form of | Myotilin (MYOT = NG_008894.1); Homo sapiens |
| limb-girdle muscular dystrophy | myotilin (MYOT), RefSeqGene (LRG_201) on |
| (LGMD1) | chromosome 5 |
| | Lamin A/C (LMNA = NG_008692.2); Homo sapiens |
| | lamin A/C (LMNA), RefSeqGene (LRG_254) on |
| | chromosome 1 |
| | Caveolin 3 (CAV3 = NG_008797.2); Homo sapiens |
| | caveolin 3 (CAV3), RefSeqGene (LRG_329) on |
| | chromosome 3 |
| | DnaJ Heat Shock Protein Family (Hsp40) Member B6 |
| | (DNAJB6 = NG_032573.1); Homo sapiens DnaJ heat |
| | shock protein family (Hsp40) member B6 (DNAJB6), |
| | RefSeqGene on chromosome 7 |
| | Desmin (DES = NG_008043.1); Homo sapiens desmin |
| | (DES), RefSeqGene (LRG_380) on chromosome 2 |
| | Transportin 3 (TNPO3 = NG_023428.1); Homo sapiens |
| | transportin 3 (TNPO3), RefSeqGene on chromosome 7 |
| | Heterogeneous nuclear ribonucleoprotein D-like |
| | (HNRNPDL= NG_029681.1); Homo sapiens |
| | heterogeneous nuclear ribonucleoprotein D like |
| | (HNRNPDL), RefSeqGene on chromosome 4 |
| Autosomal-recessive form of | Calpain 3 (CAPN3 = NG_008660.1); Homo sapiens |
| limb-girdle muscular dystrophy | calpain 3 (CAPN3), RefSeqGene (LRG_849) on |
| (LGMD2) | chromosome 15 |
| | Dysferlin (DYSF = NG_008694.1); Homo sapiens |
| | dysferlin (DYSF), RefSeqGene (LRG_845) on |
| | chromosome 2 |
| | Gamma-sarcoglycan (SGCG = NG_008759.1); Homo |
| | sapiens sarcoglycan gamma (SGCG), RefSeqGene |
| | (LRG_207) on chromosome 13 |
| | Alpha-sarcoglycan (SGCA = NG_008889.1); Homo |
| | sapiens sarcoglycan alpha (SGCA), RefSeqGene |
| | (LRG_203) on chromosome 17 |
| | Beta-sarcoglycan (SGCB = NG_008891.1); Homo |
| | sapiens sarcoglycan beta (SGCB), RefSeqGene |
| | (LRG_204) on chromosome 4 |
| | Delta-sarcoglycan (SGCD = NG_008693.2); Homo |
| | sapiens sarcoglycan delta (SGCD), RefSeqGene |
| | (LRG_205) on chromosome 5 |
| | Telethonin (TCAP = NG_008892.1); Homo sapiens titin- |
| | cap (TCAP), RefSeqGene (LRG_210) on chromosome 17 |
| | E3 ubiquitin-protein ligase TRIM32 (TRIM32 = |
| | NG_011619.1); Homo sapiens tripartite motif containing |
| | 32 (TRIM32), RefSeqGene (LRG_211) on chromosome 9 |
| | Fukutin-related protein (FKRP = NG_008898.2); Homo |
| | sapiens fukutin related protein (FKRP), RefSeqGene |
| | (LRG_761) on chromosome 19 |
| | Protein O-mannosyl-transferase 1 (POMT1 = |
| | LC030233.1); Homo sapiens POMT1 mRNA, complete |
| | cds, contains 9-bp deletion |
| | Fukutin (FKTN = NG_008754.1); Homo sapiens fukutin |
| | (FKTN), RefSeqGene (LRG_434) on chromosome 9 |
| | Protein O-mannosyl-transferase 2 (POMT2 = |
| | NG_008897.1); Homo sapiens protein O- |
| | mannosyltransferase 2 (POMT2), RefSeqGene |
| | (LRG_844) on chromosome 14 |
| | O-linked-mannose beta-1,2-N |
| | acetylglucosaminyltransferase (POMGNT1 = |
| | NG_009205.3; Homo sapiens protein O-linked mannose |
| | N-acetylglucosaminyltransferase 1 (beta 1,2-) |
| | (POMGNT1), RefSeqGene (LRG_701) on |
| | chromosome 1) |
| | Dystroglycan (DAG1 = NG_013230.4); Homo sapiens |
| | dystroglycan 1 (DAG1), RefSeqGene (LRG_854) on |
| | chromosome 3 |
| | Titin (TTN = NG_011618.3); Homo sapiens titin (TTN), |
| | RefSeqGene (LRG_391) on chromosome 2 |
| | Anoctamin 5 (ANO5 = NG_015844.1); Homo sapiens |
| | anoctamin 5 (ANO5), RefSeqGene (LRG_868) on |
| | chromosome 11 |
| | Plectin (PLEC = NG_012492.1); Homo sapiens plectin |
| | (PLEC), RefSeqGene on chromosome 8 |
| | Desmin (DES = NG_008043.1); Homo sapiens desmin |
| | (DES), RefSeqGene (LRG_380) on chromosome 2 |
| | Trafficking protein particle complex subunit 11 |
| | (TRAPPC11 = NG_033102.1); Homo sapiens trafficking |
| | protein particle complex subunit 11 (TRAPPC11), |
| | RefSeqGene on chromosome 4 |
| | Mannose-1-phosphate guanyltransferase beta (GMPPB = |
| | NG_033731.2); Homo sapiens GDP-mannose |
| | pyrophosphorylase B (GMPPB), RefSeqGene on |
| | chromosome 3 |
| | D-ribitol-5-phosphate cytidylyltransferase (ISPD) |
| | lysosomal enzyme acid alpha-glucosidase (GAA = |
| | NG_009822.1); Homo sapiens alpha glucosidase (GAA), |
| | RefSeqGene (LRG_673) on chromosome 17 |
| | LIM and senescent cell antigen-like-containing domain |
| | protein 2 (LIMS2 = NG_042235.1); Homo sapiens LIM |
| | zinc finger domain containing 2 (LIMS2), RefSeqGene on |
| | chromosome 2 |
| | Popeye-domain containing 1 (BVES = NG_046732.1); |
| | Homo sapiens blood vessel epicardial substance (BVES), |
| | RefSeqGene on chromosome 6 |
| | Lamina-associated polypeptide 1B (TOR1AIP1 = |
| | NG_042316.1); Homo sapiens torsin 1A interacting |
| | protein 1 (TOR1AIP1), RefSeqGene on chromosome 1 |
| | O-glucosyltransferase 1 (POGLUT1 = NG_034115.1); |
| | Homo sapiens protein O-glucosyltransferase 1 |
| | (POGLUT1), RefSeqGene on chromosome 3 |
| Centronuclear myopathy/ | Myotubularin (MTM1 = NG_008199.1); Homo sapiens |
| Myotubular myopathy (XLMTM) | myotubularin 1 (MTM1), RefSeqGene (LRG_839) on |
| | chromosome X |
| | Dynamin-2 (DNM2=NG_032118.1); Homo sapiens |
| | myopalladin (MYPN), RefSeqGene (LRG_410) on |
| | chromosome 10 |
| | Amphiphysin-2 (BIN1 = NG_012042.1); Homo sapiens |
| | bridging integrator 1 (BIN1), RefSeqGene (LRG_873) on |
| | chromosome 2 |
| | Ryanodine receptor channel (RYR1 = NG_008866.1); |
| | Homo sapiens ryanodine receptor 1 (RYR1), RefSeqGene |
| | (LRG_766) on chromosome 19 |
| | Titin (TTN = NG_011618.3); Homo sapiens titin (TTN), |
| | RefSeqGene (LRG_391) on chromosome 2 |
| | Striated muscle preferentially expressed protein |
| | kinase (SPEG = NG_051022.1); Homo sapiens striated |
| | muscle enriched protein kinase (SPEG), RefSeqGene on |
| | chromosome 2 |
| Metabolic myopathy (glycogen | Lysosomal enzyme acid alpha-glucosidase (GAA = |
| storage disease) | NG_009822.1); Homo sapiens alpha glucosidase (GAA), |
| (Pompe Disease) | RefSeqGene (LRG_673) on chromosome 17 |
| | Glycogen-debranching enzyme (AGL = NG_012865.1); |
| | Homo sapiens amylo-alpha-1,6-glucosidase, 4-alpha- |
| | glucanotransferase (AGL), RefSeqGene on chromosome 1 |
| | Myophosphorylase (PYGM = NG_013018.1); Homo |
| | sapiens glycogen phosphorylase, muscle associated |
| | (PYGM), RefSeqGene on chromosome 11 |
| Friedreich ataxia | Frataxin (FXN = NG_008845.2); Homo sapiens frataxin |
| | (FXN), RefSeqGene (LRG_339) on chromosome 9; |
| | nuclear gene for mitochondrial product |

### Smooth Muscle Diseases

In some embodiments, the present disclosure provides methods for treating a smooth muscle disease in a subject by delivering a complex of a therapeutic mRNA encoding a protein expressed in smooth muscle and a 3E10 antibody or antigen binding fragment thereof, as described herein, to smooth muscle tissue of the subject.

Although, in some embodiments, the polypeptide encoded by the mRNA is a wild-type version of the protein expressed in smooth muscle, it will be appreciated that naturally occurring variants or synthetically engineered versions of the protein may also find use in the compositions and methods described herein. For example, in instances where mRNA therapy is used for enzyme replacement therapy, it is common for the enzyme encoded by the mRNA to be engineered to improve enzymatic activity. Further, in certain instances, where the wild type version of a therapeutic protein is particularly large and/or includes one or more domains that are particularly susceptible to proteolytic degradation, is it common for the protein encoded by a gene therapy vector to be engineered to make the protein smaller and/or to remove susceptible regions that are dispensable for protein function.

In some embodiments, the therapeutic mRNA molecule encodes for a protein expressed in smooth muscle that is associated with a disease or disorder. For example, in some embodiments, the disease or disorder is associated with a mutation in a gene expressed in smooth muscle.

Examples of the genes expressed in smooth muscle that are associated with disease are listed in Table 4, below. Accordingly, in some embodiments, a subject with a particular disease is treated by administration of a 3E10-mRNA complex where the mRNA encodes for a polypeptide corresponding to an associated gene in Table 4. For example, in one embodiment, a mRNA molecule encoding a polypeptide associated with the Fibrillin-1 (FBN1) protein is used for the treatment of Marfan Syndrome.

TABLE 4
Example genes expressed in smooth muscle that are mutated in various diseases.

| Disease | Mutant Protein (Genes) |
|---|---|
| Thoracic aortic aneurysm and | Actin, aortic smooth muscle (ACTA2 = NG_011541.1); |
| dissections (TAAD); | Homo sapiens actin alpha 2, smooth muscle (ACTA2), |
| Moyamoya like | RefSeqGene (LRG_781) on chromosome 10 |
| cerebrovascular disease; | |
| Patent ductus arteriosus; | |
| Aortic coarctation; | |
| Pulmonary hypertension; | |
| Congenital mydriasis; | |
| Hypoperistalsis and | |
| malrotation of the gut; | |
| Hypotonic bladder | |
| Ehlers-Danlos syndrome | Collagen alpha-1(III) chain (COL3A1 = NG_007404.1); |
| (EDS) type IV | Homo sapiens collagen type III alpha 1 chain (COL3A1), |
| | RefSeqGene (LRG_3) on chromosome 2 |
| Marfan Syndrome | Fibrillin-1 (FBN1 = NG_008805.2); Homo sapiens |
| | fibrillin 1 (FBN1), RefSeqGene (LRG_778) on |
| | chromosome 15 |
| TAAD; | Microfibrillar-associated protein 5 (MFAP5 = |
| Atrial fibrillation; | NG_041814.1); Homo sapiens microfibril associated |
| mitral valve prolapse; and | protein 5 (MFAP5), RefSeqGene on chromosome 12 |
| arterial tortuosity | |
| TAAD; | Myosin-11 (MYH11 = NG_009299.1); Homo sapiens |
| Moyamoya like | myosin heavy chain 11 (MYH11), RefSeqGene |
| cerebrovascular disease; | (LRG_1401) on chromosome 16 |
| PDA | |
| Thoracic aortic aneurysm and | Myosin light chain kinase (MYLK = NG_029111.1); |
| dissections (TAAD) | Homo sapiens myosin light chain kinase (MYLK), |
| | RefSeqGene on chromosome 3 |
| TAAD; | cGMP-dependent protein kinase 1 (PRKG1 = |
| Coronary artery | NG_029982.1); Homo sapiens protein kinase cGMP- |
| aneurysm/dissection; and | dependent 1 (PRKG1), RefSeqGene (LRG_1135) on |
| arterial tortuosity | chromosome 10 |
| Loeys-Dietz syndrome; | Transforming Growth Factor Beta 2 (TGFB2 = |
| Abdominal aortic aneurysms | NG_027721.2); Homo sapiens transforming growth factor |
| and/or intracranial and other | beta 2 (TGFB2), RefSeqGene on chromosome 1 |
| arterial aneurysms and/or | |
| dissections | |
| Rienhoff syndrome; | Transforming Growth Factor Beta Receptor 3 (TGFB3 = |
| Loeys-Dietz syndrome type 5 | NG_011715.1); Homo sapiens transforming growth |
| | factor beta 3 (TGFB3), RefSeqGene (LRG_399) on |
| | chromosome 14 |
| Loeys-Dietz syndrome; | Transforming Growth Factor Beta Receptor 1 |
| Abdominal aortic aneurysms | (TGFBR1 = NG_007461.1); Homo sapiens transforming |
| and/or intracranial and other | growth factor beta receptor 1 (TGFBR1), RefSeqGene on |
| arterial aneurysms and/or | chromosome 9 |
| dissections | |
| Loeys-Dietz syndrome; | Transforming Growth Factor Beta Receptor 2 |
| Abdominal aortic aneurysms | (TGFBR2 = NG_007490.1); Homo sapiens transforming |
| and/or intracranial and other | growth factor beta receptor 2 (TGFBR2), RefSeqGene |
| arterial aneurysms and/or | (LRG_779) on chromosome 3 |
| dissections | |
| Aneurysms-osteoarthritis | Mothers against decapentaplegic homolog 3 (SMAD3 = |
| syndrome; | NG_011990.1); Homo sapiens SMAD family member 3 |
| Loeys-Dietz syndrome | (SMAD3), RefSeqGene on chromosome 15 |

### Pulmonary Diseases

In some embodiments, the present disclosure provides methods for treating a pulmonary disease in a subject by delivering a complex of a therapeutic mRNA encoding a protein expressed in the lungs and a 3E10 antibody or antigen binding fragment thereof, as described herein, to lung tissue of the subject.

Although, in some embodiments, the polypeptide encoded by the mRNA is a wild-type version of the protein expressed in lung tissue, it will be appreciated that naturally occurring variants or synthetically engineered versions of the protein may also find use in the compositions and methods described herein. For example, in instances where mRNA therapy is used for enzyme replacement therapy, it is common for the enzyme encoded by the mRNA to be engineered to improve enzymatic activity. Further, in certain instances, where the wild type version of a therapeutic protein is particularly large and/or includes one or more domains that are particularly susceptible to proteolytic degradation, is it common for the protein encoded by a gene therapy vector to be engineered to make the protein smaller and/or to remove susceptible regions that are dispensable for protein function.

In some embodiments, the therapeutic mRNA molecule encodes for a protein expressed in the lungs that is associated with a disease or disorder. For example, in some embodiments, the disease or disorder is associated with a mutation in a gene expressed in lung tissue. See, for example, Bañuls L et al., “Gene Therapy in Rare Respiratory Diseases: What Have We Learned So Far?,” Journal of Clinical Medicine, 9 (8): 2577 (2020), the disclosure of which is incorporated herein by reference, in its entirety, for all purposes.

Examples of the genes expressed in lung tissue that are associated with disease are listed in Table 5, below. Accordingly, in some embodiments, a subject with a particular disease is treated by administration of a 3E10-mRNA complex where the mRNA encodes for a polypeptide corresponding to an associated gene in Table 5. For example, in one embodiment, a mRNA molecule encoding a polypeptide associated with the Cystic fibrosis transmembrane conductance regulator (CFTR) protein is used for the treatment of Cystic fibrosis.

TABLE 5
Example genes expressed in lung tissue that are mutated in various diseases.

| Disease | Mutant Protein (Genes) |
|---|---|
| Cystic fibrosis | Cystic fibrosis transmembrane conductance regulator |
| | (CFTR = NG_016465.4); Homo sapiens CF |
| | transmembrane conductance regulator (CFTR), |
| | RefSeqGene (LRG_663) on chromosome 7 |
| alpha-1 antitrypsin deficiency | Alpha-1-antitrypsin (SERPINA1 = NG_008290.1); |
| (AATD) | Homo sapiens serpin family A member 1 (SERPINA1), |
| | RefSeqGene (LRG_575) on chromosome 14 |
| Primary ciliary dyskinesia (PCD) | Dynein axonemal heavy chain 5 (DNAH5 = |
| | NG_013081.2); Homo sapiens dynein axonemal heavy |
| | chain 5 (DNAH5), RefSeqGene on chromosome 5 |
| | Dynein axonemal intermediate chain 1 (DNAI1 = |
| | NG_008127.1); Homo sapiens dynein axonemal |
| | intermediate chain 1 (DNAI1), RefSeqGene on |
| | chromosome 9 |
| | Dynein axonemal heavy chain 11 (DNAH11 = |
| | NG_012886.2); Homo sapiens dynein axonemal heavy |
| | chain 11 (DNAH11), RefSeqGene on chromosome 7 |
| | Dynein axonemal intermediate chain 2 (DNAI2 = |
| | NG_016865.1); Homo sapiens dynein axonemal |
| | intermediate chain 2 (DNAI2), RefSeqGene on |
| | chromosome 17 |

### Brain Diseases and Diseases of the Central Nervous System (CNS)

In some embodiments, the present disclosure provides methods for treating a brain and/or CNS disease in a subject by delivering a complex of a therapeutic mRNA encoding a protein expressed in the brain and/or CNS and a 3E10 antibody or antigen binding fragment thereof, as described herein, to brain and/or CNS tissue of the subject.

Although, in some embodiments, the polypeptide encoded by the mRNA is a wild-type version of the protein expressed in brain and/or CNS tissue, it will be appreciated that naturally occurring variants or synthetically engineered versions of the protein may also find use in the compositions and methods described herein. For example, in instances where mRNA therapy is used for enzyme replacement therapy, it is common for the enzyme encoded by the mRNA to be engineered to improve enzymatic activity. Further, in certain instances, where the wild type version of a therapeutic protein is particularly large and/or includes one or more domains that are particularly susceptible to proteolytic degradation, is it common for the protein encoded by a gene therapy vector to be engineered to make the protein smaller and/or to remove susceptible regions that are dispensable for protein function.

In some embodiments, the therapeutic mRNA molecule encodes for a protein expressed in the brain and/or CNS that is associated with a disease or disorder. For example, in some embodiments, the disease or disorder is associated with a mutation in a gene expressed in brain and/or CNS tissue. See, for example, Ehrhart, F. et al., “A resource to explore the discovery of rare diseases and their causative genes,” Sci Data 8, 124 (2021), which curates the genes responsible for 4166 rare monogenic diseases, the disclosure of which is incorporated herein by reference, in its entirety, for all purposes.

Examples of the genes expressed in the brain and/or CNS that are associated with disease are listed in Table 6, below. Accordingly, in some embodiments, a subject with a particular disease is treated by administration of a 3E10-mRNA complex where the mRNA encodes for a polypeptide corresponding to an associated gene in Table 6. For example, in one embodiment, a mRNA molecule encoding a polypeptide associated with the Synaptic functional regulator FMR1 (FMR1) protein is used for the treatment of Fragile X syndrome.

TABLE 6
Example genes expressed in brain and/or CNS tissue that are mutated
in various diseases.

| Disease | Mutant Protein (Genes) |
|---|---|
| Giant Axonal Neuropathy (GAN) | Gigaxonin (GAN = NG_009007.1); Homo sapiens |
| | gigaxonin (GAN), RefSeqGene (LRG_242) on |
| | chromosome 16 |
| GM2 gangliosidosis | Beta-hexosaminidase subunit alpha (HEXA = |
| | NG_009017.2); Homo sapiens hexosaminidase subunit |
| | alpha (HEXA), RefSeqGene on chromosome 15 |
| | Beta-hexosaminidase subunit beta (HEXB = |
| | NG_009770.2); Homo sapiens hexosaminidase subunit |
| | beta (HEXB), RefSeqGene on chromosome 5; nuclear |
| | gene for mitochondrial product |
| Ceroid lipofuscinosis, neuronal, 1 | Palmitoyl-protein thioesterase 1 (PPT1 = |
| (CLN1) disease | NG_009192.1); Homo sapiens palmitoyl-protein |
| | thioesterase 1 (PPT1), RefSeqGene (LRG_690) on |
| | chromosome 1 |
| GM2-gangliosidosis, AB variant | Ganglioside GM2 activator (GM2A = NG_009059.1); |
| | Homo sapiens GM2 ganglioside activator (GM2A), |
| | RefSeqGene on chromosome 5 |
| Leigh syndrome | Surfeit locus protein 1 (SURF1 = NG_008477.1); Homo |
| | sapiens SURF1 cytochrome c oxidase assembly factor |
| | (SURF1), RefSeqGene on chromosome 9 |
| Rett syndrome | Methyl-CpG-binding protein 2 (MECP2; |
| | NG_007107.3); Homo sapiens methyl-CpG binding |
| | protein 2 (MECP2), RefSeqGene (LRG_764) on |
| | chromosome X |
| Fragile X syndrome | Synaptic functional regulator FMR1 (FMR1 = |
| | NG_007529.2); Homo sapiens FMRP translational |
| | regulator 1 (FMR1), RefSeqGene (LRG_762) on |
| | chromosome X |
| SLC6A1 haploinsufficiency | Sodium- and chloride-dependent GABA transporter 1 |
| disorder | (SLC6A1 = NG_053003.1); Homo sapiens solute carrier |
| | family 6 member 1 (SLC6A1), RefSeqGene on |
| | chromosome 3 |
| SLC13A5 deficiency | Solute carrier family 13 member 5 (SLC13A5 = |
| | NG_034220.1); Homo sapiens solute carrier family 13 |
| | member 5 (SLC13A5), RefSeqGene (LRG_1020) on |
| | chromosome 17 |
| Friedreich ataxia | Frataxin (FXN = NG_008845.2); Homo sapiens frataxin |
| | (FXN), RefSeqGene (LRG_339) on chromosome 9; |
| | nuclear gene for mitochondrial product |
| Autosomal recessive cerebellar | Spectrin Repeat Containing Nuclear Envelope Protein 1 |
| ataxia type 1 (ARCA1) | (SYNE1 = NG_012855.2); Homo sapiens spectrin |
| | repeat containing nuclear envelope protein 1 (SYNE1), |
| | RefSeqGene (LRG_427) on chromosome 6 |

### Diseases of the Diaphragm

In some embodiments, the present disclosure provides methods for treating a disease of the diaphragm in a subject by delivering a complex of a therapeutic mRNA encoding a protein expressed in the diaphragm and a 3E10 antibody or antigen binding fragment thereof, as described herein, to the diaphragm of the subject.

Although, in some embodiments, the polypeptide encoded by the mRNA is a wild-type version of the protein expressed in the diaphragm, it will be appreciated that naturally occurring variants or synthetically engineered versions of the protein may also find use in the compositions and methods described herein. For example, in instances where mRNA therapy is used for enzyme replacement therapy, it is common for the enzyme encoded by the mRNA to be engineered to improve enzymatic activity. Further, in certain instances, where the wild type version of a therapeutic protein is particularly large and/or includes one or more domains that are particularly susceptible to proteolytic degradation, is it common for the protein encoded by a gene therapy vector to be engineered to make the protein smaller and/or to remove susceptible regions that are dispensable for protein function.

In some embodiments, the therapeutic mRNA molecule encodes for a protein expressed in the diaphragm that is associated with a disease or disorder. For example, in some embodiments, the disease or disorder is associated with a mutation in a gene expressed in the diaphragm.

Examples of the genes expressed in the diaphragm that are associated with disease are listed in Table 7, below. Accordingly, in some embodiments, a subject with a particular disease is treated by administration of a 3E10-mRNA complex where the mRNA encodes for a polypeptide corresponding to an associated gene in Table 7. For example, in one embodiment, a mRNA molecule encoding a polypeptide associated with the myotubularin (MTM1) protein is used for the treatment of X-linked myotubular myopathy.

TABLE 7
Example genes expressed in diaphragm tissue that are mutated in
various diseases.

| Disease | Mutant Protein (Genes) |
|---|---|
| Duchenne and Becker | Dystrophin (DMD = NG_012232.1; Homo sapiens |
| muscular dystrophy | dystrophin (DMD), RefSeqGene (LRG_199) on |
| | chromosome X) |
| Autosomal-dominant form of | Myotilin (MYOT = NG_008894.1); Homo sapiens |
| limb-girdle muscular dystrophy | myotilin (MYOT), RefSeqGene (LRG_201) on |
| (LGMD1) | chromosome 5 |
| | Lamin A/C (LMNA = NG_008692.2); Homo sapiens |
| | lamin A/C (LMNA), RefSeqGene (LRG_254) on |
| | chromosome 1 |
| | Caveolin 3 (CAV3 = NG_008797.2); Homo sapiens |
| | caveolin 3 (CAV3), RefSeqGene (LRG_329) on |
| | chromosome 3 |
| | DnaJ Heat Shock Protein Family (Hsp40) Member B6 |
| | (DNAJB6 = NG_032573.1); Homo sapiens DnaJ heat |
| | shock protein family (Hsp40) member B6 (DNAJB6), |
| | RefSeqGene on chromosome 7 |
| | Desmin (DES = NG_008043.1); Homo sapiens desmin |
| | (DES), RefSeqGene (LRG_380) on chromosome 2 |
| | Transportin 3 (TNPO3 = NG_023428.1); Homo sapiens |
| | transportin 3 (TNPO3), RefSeqGene on chromosome 7 |
| | Heterogeneous nuclear ribonucleoprotein D-like |
| | (HNRNPDL= NG_029681.1); Homo sapiens |
| | heterogeneous nuclear ribonucleoprotein D like |
| | (HNRNPDL), RefSeqGene on chromosome 4 |
| Autosomal-recessive form of | Calpain 3 (CAPN3 = NG_008660.1); Homo sapiens |
| limb-girdle muscular dystrophy | calpain 3 (CAPN3), RefSeqGene (LRG_849) on |
| (LGMD2) | chromosome 15 |
| | Dysferlin (DYSF = NG_008694.1); Homo sapiens |
| | dysferlin (DYSF), RefSeqGene (LRG_845) on |
| | chromosome 2 |
| | Gamma-sarcoglycan (SGCG = NG_008759.1); Homo |
| | sapiens sarcoglycan gamma (SGCG), RefSeqGene |
| | (LRG_207) on chromosome 13 |
| | Alpha-sarcoglycan (SGCA = NG_008889.1); Homo |
| | sapiens sarcoglycan alpha (SGCA), RefSeqGene |
| | (LRG_203) on chromosome 17 |
| | Beta-sarcoglycan (SGCB = NG_008891.1); Homo |
| | sapiens sarcoglycan beta (SGCB), RefSeqGene |
| | (LRG_204) on chromosome 4 |
| | Delta-sarcoglycan (SGCD = NG_008693.2); Homo |
| | sapiens sarcoglycan delta (SGCD), RefSeqGene |
| | (LRG_205) on chromosome 5 |
| | Telethonin (TCAP = NG_008892.1); Homo sapiens titin- |
| | cap (TCAP), RefSeqGene (LRG_210) on chromosome 17 |
| | E3 ubiquitin-protein ligase TRIM32 (TRIM32 = |
| | NG_011619.1); Homo sapiens tripartite motif containing |
| | 32 (TRIM32), RefSeqGene (LRG_211) on chromosome 9 |
| | Fukutin-related protein (FKRP = NG_008898.2); Homo |
| | sapiens fukutin related protein (FKRP), RefSeqGene |
| | (LRG_761) on chromosome 19 |
| | Protein O-mannosyl-transferase 1 (POMT1 = |
| | LC030233.1); Homo sapiens POMT1 mRNA, complete |
| | cds, contains 9-bp deletion |
| | Fukutin (FKTN = NG_008754.1); Homo sapiens fukutin |
| | (FKTN), RefSeqGene (LRG_434) on chromosome 9 |
| | Protein O-mannosyl-transferase 2 (POMT2 = |
| | NG_008897.1); Homo sapiens protein O- |
| | mannosyltransferase 2 (POMT2), RefSeqGene |
| | (LRG_844) on chromosome 14 |
| | O-linked-mannose beta-1,2-N |
| | acetylglucosaminyltransferase (POMGNT1 = |
| | NG_009205.3; Homo sapiens protein O-linked mannose |
| | N-acetylglucosaminyltransferase 1 (beta 1,2-) |
| | (POMGNT1), RefSeqGene (LRG_701) on |
| | chromosome 1) |
| | Dystroglycan (DAG1 = NG_013230.4); Homo sapiens |
| | dystroglycan 1 (DAG1), RefSeqGene (LRG_854) on |
| | chromosome 3 |
| | Titin (TTN = NG_011618.3); Homo sapiens titin (TTN), |
| | RefSeqGene (LRG_391) on chromosome 2 |
| | Anoctamin 5 (ANO5 = NG_015844.1); Homo sapiens |
| | anoctamin 5 (ANO5), RefSeqGene (LRG_868) on |
| | chromosome 11 |
| | Plectin (PLEC = NG_012492.1); Homo sapiens plectin |
| | (PLEC), RefSeqGene on chromosome 8 |
| | Desmin (DES = NG_008043.1); Homo sapiens desmin |
| | (DES), RefSeqGene (LRG_380) on chromosome 2 |
| | Trafficking protein particle complex subunit 11 |
| | (TRAPPC11 = NG_033102.1); Homo sapiens trafficking |
| | protein particle complex subunit 11 (TRAPPC11), |
| | RefSeqGene on chromosome 4 |
| | Mannose-1-phosphate guanyltransferase beta (GMPPB = |
| | NG_033731.2); Homo sapiens GDP-mannose |
| | pyrophosphorylase B (GMPPB), RefSeqGene on |
| | chromosome 3 |
| | D-ribitol-5-phosphate cytidylyltransferase (ISPD) |
| | lysosomal enzyme acid alpha-glucosidase (GAA = |
| | NG_009822.1); Homo sapiens alpha glucosidase (GAA), |
| | RefSeqGene (LRG_673) on chromosome 17 |
| | LIM and senescent cell antigen-like-containing domain |
| | protein 2 (LIMS2 = NG_042235.1); Homo sapiens LIM |
| | zinc finger domain containing 2 (LIMS2), RefSeqGene on |
| | chromosome 2 |
| | Popeye-domain containing 1 (BVES = NG_046732.1); |
| | Homo sapiens blood vessel epicardial substance (BVES), |
| | RefSeqGene on chromosome 6 |
| | Lamina-associated polypeptide 1B (TOR1AIP1 = |
| | NG_042316.1); Homo sapiens torsin 1A interacting |
| | protein 1 (TOR1AIP1), RefSeqGene on chromosome 1 |
| | O-glucosyltransferase 1 (POGLUT1 = NG_034115.1); |
| | Homo sapiens protein O-glucosyltransferase 1 |
| | (POGLUT1), RefSeqGene on chromosome 3 |
| Centronuclear myopathy/ | Myotubularin (MTM1 = NG_008199.1); Homo sapiens |
| Myotubular myopathy (XLMTM) | myotubularin 1 (MTM1), RefSeqGene (LRG_839) on |
| | chromosome X |
| | Dynamin-2 (DNM2=NG_032118.1); Homo sapiens |
| | myopalladin (MYPN), RefSeqGene (LRG_410) on |
| | chromosome 10 |
| | Amphiphysin-2 (BIN1 = NG_012042.1); Homo sapiens |
| | bridging integrator 1 (BIN1), RefSeqGene (LRG_873) on |
| | chromosome 2 |
| | Ryanodine receptor channel (RYR1 = NG_008866.1); |
| | Homo sapiens ryanodine receptor 1 (RYR1), RefSeqGene |
| | (LRG_766) on chromosome 19 |
| | Titin (TTN = NG_011618.3); Homo sapiens titin (TTN), |
| | RefSeqGene (LRG_391) on chromosome 2 |
| | Striated muscle preferentially expressed protein |
| | kinase (SPEG = NG_051022.1); Homo sapiens striated |
| | muscle enriched protein kinase (SPEG), RefSeqGene on |
| | chromosome 2 |
| Metabolic myopathy | Lysosomal enzyme acid alpha-glucosidase (GAA = |
| (glycogen storage disease) | NG_009822.1); Homo sapiens alpha glucosidase (GAA), |
| (Pompe Disease) | RefSeqGene (LRG_673) on chromosome 17 |
| | Glycogen-debranching enzyme (AGL = NG_012865.1); |
| | Homo sapiens amylo-alpha-1,6-glucosidase, 4-alpha- |
| | glucanotransferase (AGL), RefSeqGene on chromosome 1 |
| | Myophosphorylase (PYGM = NG_013018.1); Homo |
| | sapiens glycogen phosphorylase, muscle associated |
| | (PYGM), RefSeqGene on chromosome 11 |

### Hypertrophic Cardiomyopathy (HCM)

One such disorder for which a gene therapy is being developed is hypertrophic cardiomyopathy (HCM) caused by MYBPC3 mutations in patients. HCM is one of the most common genetic heart diseases, with about 500,000 patients diagnosed with HCM worldwide. Up to 60% of HCM cases have a genetic origin, and it is estimated that 40% of those have mutations in MYBPC3, the gene that encodes cardiac myosin-binding protein C (MyBP-C). Gene replacement therapies have aimed to increase cMyBP-C protein in mouse models harboring Mybpc3 truncating variants that lead to haploinsufficiency. Lentiviral delivery of full-length mouse Mypbc3 cDNA to the myocardium of homozygous Mybpc3-null mice restored normal protein content and cross-bridge kinetics in isolated heart tissues, and in vivo assessments revealed improved ventricular function (Harris S P et al. Hypertrophic cardiomyopathy in cardiac myosin binding protein-C knockout mice. Circ Res. 2002; 90:594-601). A related approach used AAV9 carrying the mouse Mybpc3-cDNA that was expressed under the control of the troponin promoter (Mearini G et al. Mybpc3 gene therapy for neonatal cardiomyopathy enables long-term disease prevention in mice. Nat Commun. 2014; 5:5515, the content of which is incorporated herein by reference). A single injection into neonatal homozygous LoF Mybpc3-mutant mice both restored normal cMyBP-C protein levels and suppressed low-level production of defective mRNA species from the mutant alleles. This strategy prevented the development of HCM for approximately 6 months, after which suppression was lost, presumably because of decline in AAV9 titers.

### Hemophilia

Hemophilia is an X-linked inherited bleeding disorder affecting approximately 196 706 persons globally (World Federation of Hemophilia (2018) Report on the Annual Global Survey 2017. WFH, Montreal, Canada, the content of which is incorporated herein by reference). Hemophilia is caused by mutations in the F8 (hemophilia A) or F9 (hemophilia B) genes, resulting in reduced production/function of the Factor VIII (FVIII) or Factor IX (FIX) proteins. Patients with severe hemophilia have an absence of circulating plasma FVIII or FIX activity (<1%), resulting in spontaneous bleeding affecting the joints and soft tissues. Without treatment, recurrent bleeding results in the development of chronic arthropathy (knee, ankle and elbow) and early mortality.

The current standard of care for persons with hemophilia involves regular self-infusion (prophylaxis) of intravenous concentrates of exogenously derived FVIII or FIX. The aim of prophylaxis is to raise FVIII or FIX activity above a level that is detectable (>1%) to prevent bleeding and reduce or delay the incidence of joint disease (Manco-Johnson, M. J. et al. (2007) Prophylaxis versus episodic treatment to prevent joint disease in boys with severe hemophilia. N. Engl. J. Med, 357, 535-544).

Hemophilia provides an attractive target for gene therapy studies, due to the monogenic nature of these disorders and easily measurable endpoints, e.g., measurement of factor levels and bleed rates). Thus far, all successful, pre-clinical and clinical studies have utilized recombinant adeno-associated viral (AAV) vectors for Factor VIII or Factor IX hepatocyte transduction and the clinical data have presented normalization of factor levels in some patients with improvements in bleed rate. The main toxicity seen, however, has been early transient elevation in liver enzymes, with variable effect on transgene expression.

In some embodiments, the therapeutic mRNA molecule encodes for Factor VIII (FVIII) or Factor IX (FIX) proteins.

### Alpha-1 Antitrypsin (AAT) Deficiency

Alpha-1 antitrypsin (AAT) deficiency, characterized by low plasma levels of the serine protease inhibitor AAT, is associated with emphysema secondary to insufficient protection of the lung from neutrophil proteases. Although AAT augmentation therapy with purified AAT protein is efficacious, it requires weekly to monthly intravenous infusion of AAT purified from pooled human plasma, has the risk of viral contamination and allergic reactions, and is costly.

AAT, is a serum serine protease inhibitor and functions to protect the lungs from the activity of the powerful protease neutrophil elastase (NE). In addition to its role in regulating NE, AAT inhibits the activity of other neutrophil-released proteases, including proteinase 3, α-defensins, and cathepsin G, and has anti-inflammatory and immunomodulatory properties. AAT is produced mainly in the liver (hepatocytes), reaching the lung by diffusion from the circulation, with a small percentage secreted locally from mononuclear phagocytes, neutrophils, bronchial epithelial cells, and small intestine epithelial cells).

A gene therapy study for ATT deficiency was completed in a dog model after autologous transplant of retroviral transduced hepatocytes (Kay M A et al. Expression of human alpha 1-antitrypsin in dogs after autologous transplantation of retroviral transduced hepatocytes. Proc Natl Acad Sci USA. 1992; 89:89-93). Primary hepatocytes were isolated from the animals, transduced with a retroviral vector containing the human AAT cDNA, and transplanted into dogs by administration through the spleen. Human AAT was detected transiently in serum for up to 1 month, reaching the maximal level of 4.6 μg/ml around 1 week after transplant.

Accordingly, in one aspect, the present disclosure provides methods for treating a hepatic disease or disorder in a subject by delivering a complex of a therapeutic mRNA encoding a hepatic protein and a 3E10 antibody or antigen binding fragment thereof, as described herein, to hepatic cells of the subject.

Although, in some embodiments, the polypeptide encoded by the mRNA is a wild-type version of the hepatic protein, it will be appreciated that naturally occurring variants or synthetically engineered versions of a hepatic protein may also find use in the compositions and methods described herein. For example, in instances where mRNA therapy is used for enzyme replacement therapy, it is common for the enzyme encoded by the mRNA to be engineered to improve enzymatic activity. Further, in certain instances, where the wild type version of a therapeutic protein is particularly large and/or includes one or more domains that are particularly susceptible to proteolytic degradation, is it common for the protein encoded by a gene therapy vector to be engineered to make the protein smaller and/or to remove susceptible regions that are dispensable for protein function.

In some embodiments, the therapeutic mRNA molecule encodes for a hepatic protein.

In some embodiments, the therapeutic mRNA molecule encodes for Alpha-1 antitrypsin (AAT) protein.

In some embodiments, the subject has a genetic hepatic disease. For example, in some embodiments, the subject carries a hepatic gene having a partial or complete loss-of-function mutation. Accordingly, in some embodiments, the therapeutic mRNA administered to the subject encodes for a functional copy of a polypeptide corresponding to the mutated gene in the subject. However, in some instances, the mRNA encodes for a homologue of the protein encoded by the mutant gene in the subject, a protein that has partially redundant function, and/or a protein that functions in a partially redundant pathway as the protein encoded by the mutant gene in the subject.

### Cystic Fibrosis

Cystic Fibrosis (CF) is the most common life-limiting fatal genetic disorder, affecting approximately 90,000 individuals worldwide. It is an autosomal recessive disorder that requires mutations in the CF gene in both genetic alleles. The CF gene encodes for a protein the cystic fibrosis transmembrane conductance regulator (CFTR), which is a protein chloride channel, belongs to the family of adenosine triphosphate (ATP)-binding cassette (ABC) transporters.

Mutations in the CF gene affecting CFTR expression, protein levels, or function, e.g., CFTR variants, affect multiple organ systems including the lung, pancreas, liver, gut, and reproductive organs. Changes in chloride and bicarbonate transportation across this channel impairs epithelial cell functions including mucociliary transport of foreign agents out of the airways, elevated concentrations sweat chloride, impairment in pancreatic hormone regulation, and intestinal obstruction (Kopito R R. Biosynthesis and degradation of CFTR. Physiol. Rev. 1999; 79 (1 Suppl): S167-73).

Gene therapy offers an opportunity for the treatment of cystic fibrosis by replacing the genetic mutation with a “correct version” of the CFTR gene. Currently, there is an ongoing Phase I and II clinical trial for MRT5005, a drug that delivers CFTR-encoded mRNA to the lungs (Sponsor is Translate Bio, previously RaNA Therapeutics).

Accordingly, in one aspect, the present disclosure provides methods for treating a lung disease or disorder in a subject by delivering a complex of a therapeutic mRNA encoding a lung protein and a 3E10 antibody or antigen binding fragment thereof, as described herein, to lung epithelial cells of the subject.

Although, in some embodiments, the polypeptide encoded by the mRNA is a wild-type version of the lung protein, it will be appreciated that naturally occurring variants or synthetically engineered versions of a lung protein may also find use in the compositions and methods described herein. For example, in instances where mRNA therapy is used for enzyme replacement therapy, it is common for the enzyme encoded by the mRNA to be engineered to improve enzymatic activity. Further, in certain instances, where the wild type version of a therapeutic protein is particularly large and/or includes one or more domains that are particularly susceptible to proteolytic degradation, is it common for the protein encoded by a gene therapy vector to be engineered to make the protein smaller and/or to remove susceptible regions that are dispensable for protein function.

In some embodiments, the therapeutic mRNA molecule encodes for a lung protein.

In some embodiments, the therapeutic mRNA molecule encodes for cystic fibrosis transmembrane conductance regulator (CFTR) protein.

In some embodiments, the subject has a genetic lung disease. For example, in some embodiments, the subject carries a lung gene having a partial or complete loss-of-function mutation. Accordingly, in some embodiments, the therapeutic mRNA administered to the subject encodes for a functional copy of a polypeptide corresponding to the mutated gene in the subject. However, in some instances, the mRNA encodes for a homologue of the protein encoded by the mutant gene in the subject, a protein that has partially redundant function, and/or a protein that functions in a partially redundant pathway as the protein encoded by the mutant gene in the subject.

### Spinal Muscular Atrophy (SMA)

SMA is characterized by degeneration of spinal cord alpha motor neurons resulting in muscular wasting. The disease impairs the patient's ability to walk, speak and breathe. It affects approximately 10 in 100.000 newborns and is the most common monogenic disease leading to death in infants. SMA is the result of a loss of function (LoF) mutation in the survival of motor-neuron 1 (SMN1) gene SMN1 encodes a protein essential for survival of the alpha motor neurons.

The first gene therapy for SMA, onasemnogene abeparvovec (AVXS-101), developed by AveXis (acquired by Novartis Pharmaceuticals), was approved under the brand name Zolgensma® by the FDA and EMA in 2019 and 2020, respectively. AVXS-101 is an SMN1 gene replacement therapy delivered by a self-complementary AAV9 (scAAV9) virus that is able to cross the blood brain barrier (BBB). It has a constitutively active promoter providing persistent expression of SMN1 protein.

Accordingly, in one aspect, the present disclosure provides methods for treating a brain disease or disorder in a subject by delivering a complex of a therapeutic mRNA encoding a neuronal protein and a 3E10 antibody or antigen binding fragment thereof, as described herein, to a neuronal cell of the subject.

Although, in some embodiments, the polypeptide encoded by the mRNA is a wild-type version of the neuronal protein, it will be appreciated that naturally occurring variants or synthetically engineered versions of a neuronal protein may also find use in the compositions and methods described herein. For example, in instances where mRNA therapy is used for enzyme replacement therapy, it is common for the enzyme encoded by the mRNA to be engineered to improve enzymatic activity. Further, in certain instances, where the wild type version of a therapeutic protein is particularly large and/or includes one or more domains that are particularly susceptible to proteolytic degradation, is it common for the protein encoded by a gene therapy vector to be engineered to make the protein smaller and/or to remove susceptible regions that are dispensable for protein function.

In some embodiments, the therapeutic mRNA molecule encodes for a neuronal protein.

In some embodiments, the therapeutic mRNA molecule encodes for survival of motor-neuron 1 (SMN1) protein.

In some embodiments, the subject has a genetic brain disease. For example, in some embodiments, the subject carries a neuronal gene having a partial or complete loss-of-function mutation. Accordingly, in some embodiments, the therapeutic mRNA administered to the subject encodes for a functional copy of a polypeptide corresponding to the mutated gene in the subject. However, in some instances, the mRNA encodes for a homologue of the protein encoded by the mutant gene in the subject, a protein that has partially redundant function, and/or a protein that functions in a partially redundant pathway as the protein encoded by the mutant gene in the subject.

### Compositions for Treating Genetic Diseases

In one aspect, the present disclosure provides pharmaceutical compositions including a complex formed between a therapeutic mRNA polynucleotide encoding a cardiac muscle polypeptide, as described herein, and a 3E10 antibody or antigen binding fragment thereof, as described herein.

In another aspect, the present disclosure provides pharmaceutical compositions including a complex formed between a therapeutic mRNA polynucleotide encoding a hepatic polypeptide, as described herein, and a 3E10 antibody or antigen binding fragment thereof, as described herein.

In one aspect, the present disclosure provides pharmaceutical compositions including a complex formed between a therapeutic mRNA polynucleotide encoding a lung polypeptide, as described herein, and a 3E10 antibody or antigen binding fragment thereof, as described herein.

In one aspect, the present disclosure provides pharmaceutical compositions including a complex formed between a therapeutic mRNA polynucleotide encoding a neuronal polypeptide, as described herein, and a 3E10 antibody or antigen binding fragment thereof, as described herein.

In some embodiments, a pharmaceutical composition described herein has a molar ratio of 3E10 antibody or antigen binding fragment thereof to therapeutic mRNA of at least 2:1. As reported in Examples 3 and 6, the use of molar ratios of 3E10 antibody or antigen binding fragment thereof to mRNAs molecules in the compositions described herein protects the mRNA molecule from RNA degradation.

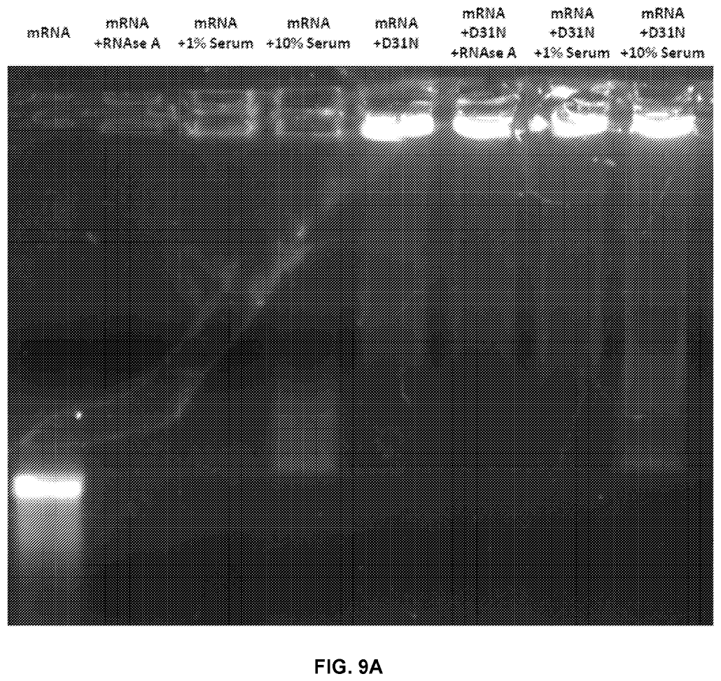

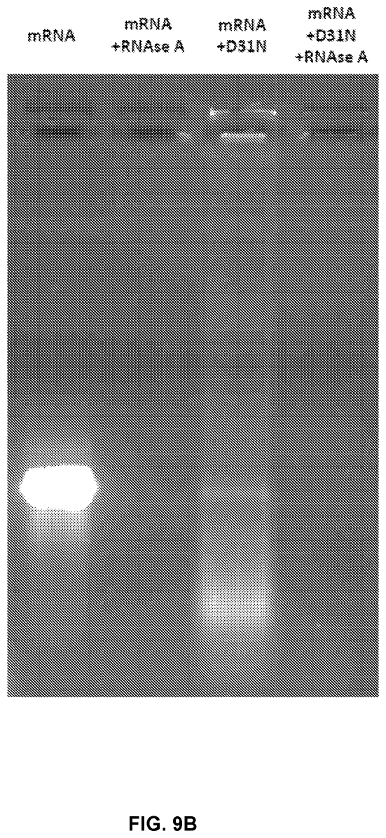

Further, as illustrated in FIGS. 9A and 9B, while parental 3E10 antibodies protected mRNA from RNAse A-mediated RNA degradation at molar ratios of 2:1 and 20:1, the protection afforded by the 20:1 molar ratio exceeded the protection afforded at 2:1. Accordingly, in some embodiments, a pharmaceutical composition described herein has a molar ratio of 3E10 antibody or antigen binding fragment thereof to therapeutic polynucleotide that is at least about 2:1. In some embodiments, a pharmaceutical composition described herein has a molar ratio of 3E10 antibody or antigen binding fragment thereof to therapeutic polynucleotide that is at least about 5:1. In some embodiments, a pharmaceutical composition described herein has a molar ratio of 3E10 antibody or antigen binding fragment thereof to therapeutic polynucleotide that is at least about 7.5:1. In some embodiments, a pharmaceutical composition described herein has a molar ratio of 3E10 antibody or antigen binding fragment thereof to therapeutic polynucleotide that is at least about 10:1. In some embodiments, a pharmaceutical composition described herein has a molar ratio of 3E10 antibody or antigen binding fragment thereof to therapeutic polynucleotide that is at least about 15:1. In some embodiments, a pharmaceutical composition described herein has a molar ratio of 3E10 antibody or antigen binding fragment thereof to therapeutic polynucleotide that is at least about 20:1. In some embodiments, a pharmaceutical composition described herein has a molar ratio of 3E10 antibody or antigen binding fragment thereof to therapeutic polynucleotide that is at least about 25:1. In some embodiments, a pharmaceutical composition described herein has a molar ratio of 3E10 antibody or antigen binding fragment thereof to therapeutic polynucleotide that is at least about 30:1. In some embodiments, a pharmaceutical composition described herein has a molar ratio of 3E10 antibody or antigen binding fragment thereof to therapeutic polynucleotide that is at least about 40:1. In some embodiments, a pharmaceutical composition described herein has a molar ratio of 3E10 antibody or antigen binding fragment thereof to therapeutic polynucleotide that is at least about 50:1.

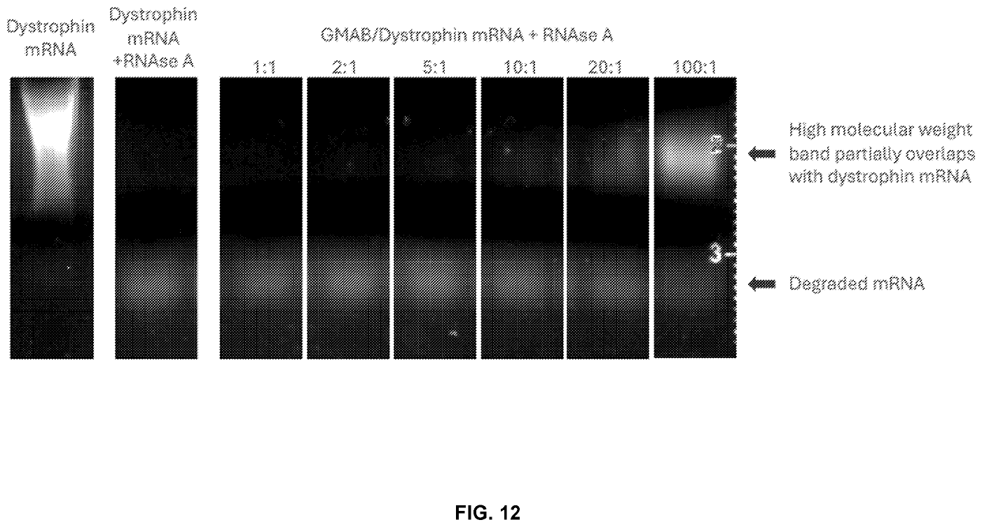

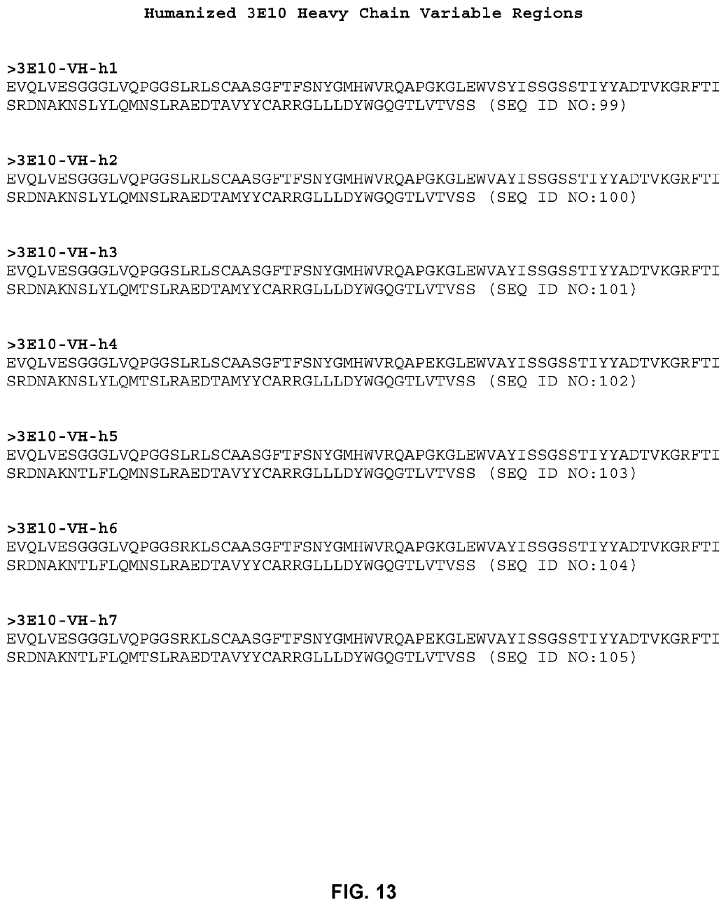

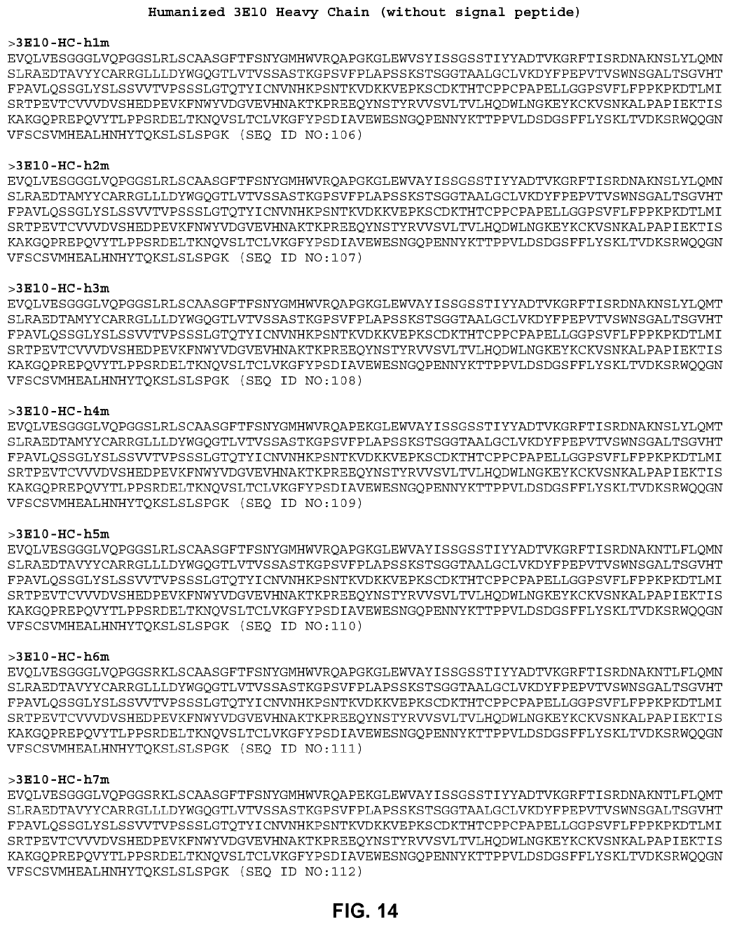

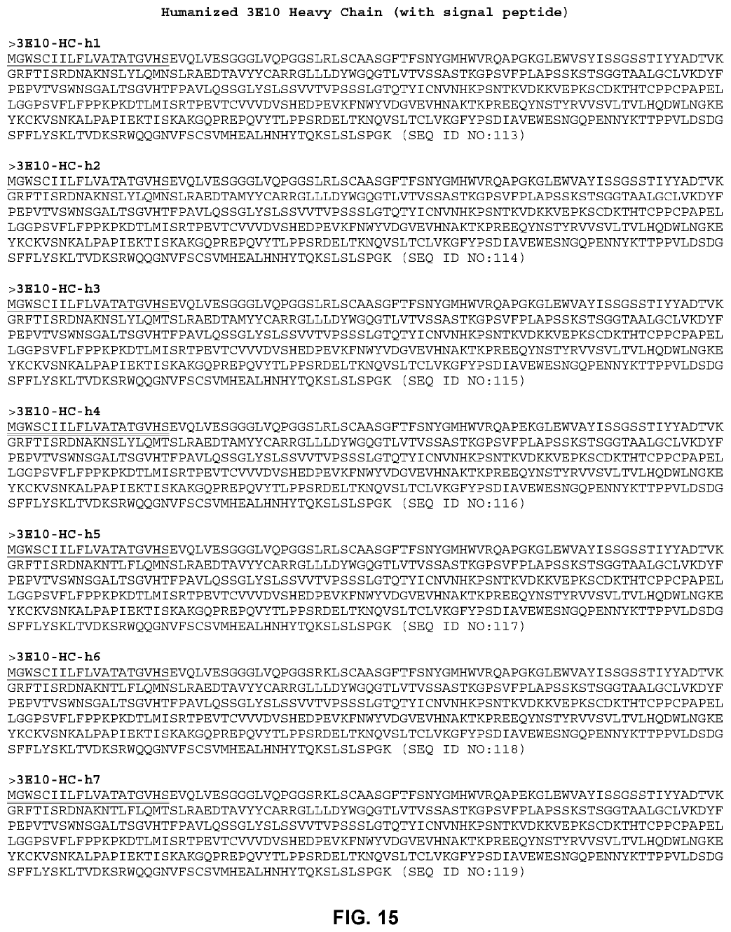

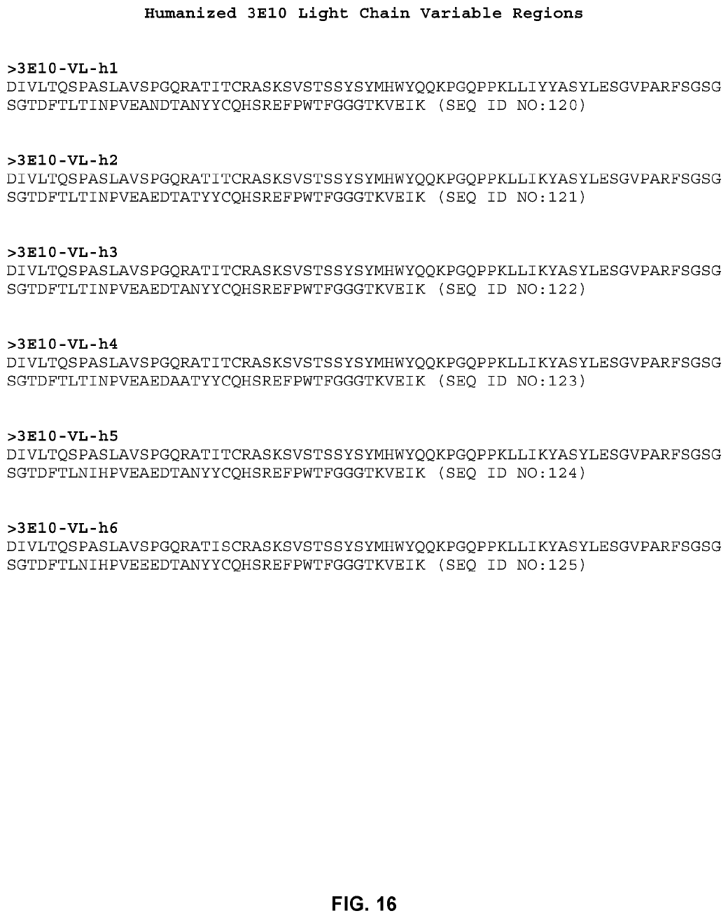

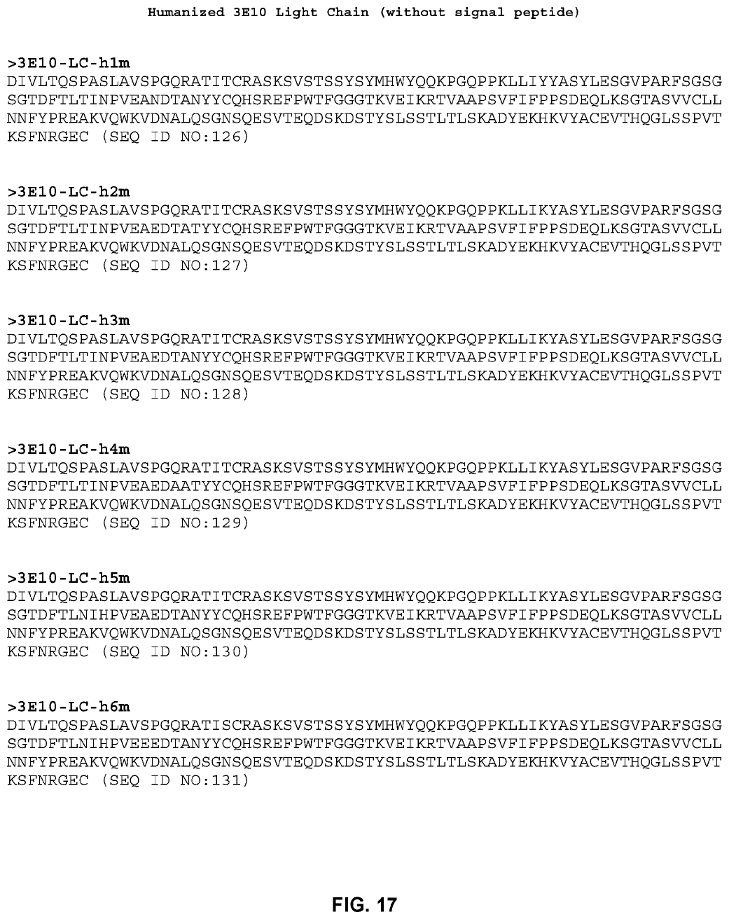

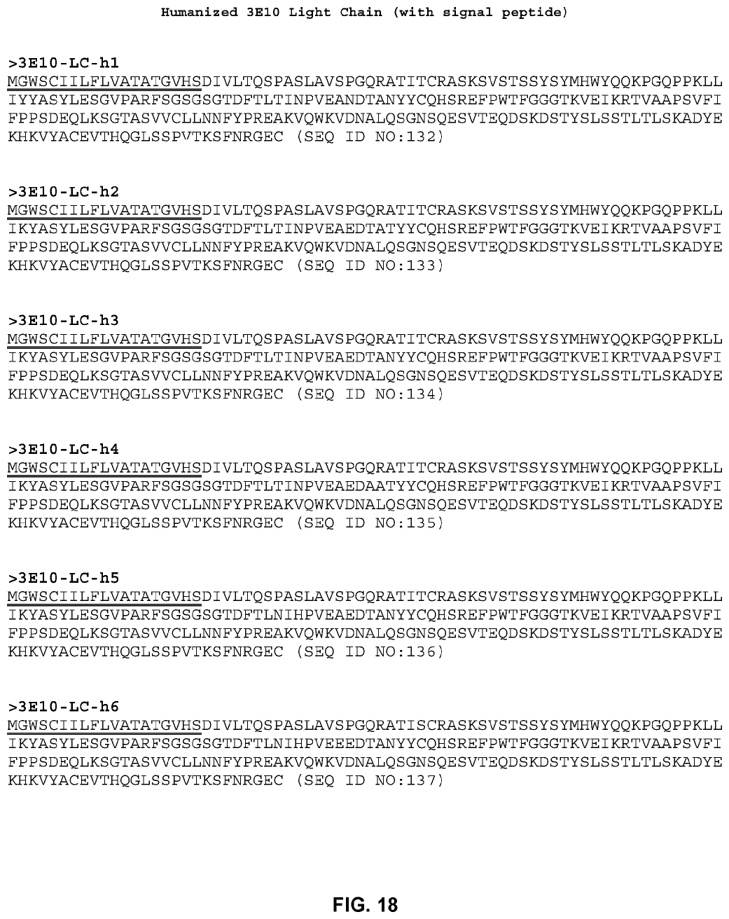

Further, as shown in FIG. 12, the use of higher stoichiometric ratios better protect longer polynucleotides from degradation. Accordingly, in some embodiments, a pharmaceutical composition described herein has a molar ratio of 3E10 antibody or antigen binding fragment thereof to therapeutic polynucleotide that is at least about 50:1. In some embodiments, a pharmaceutical composition described herein has a molar ratio of 3E10 antibody or antigen binding fragment thereof to therapeutic polynucleotide that is at least about 75:1. In some embodiments, a pharmaceutical composition described herein has a molar ratio of 3E10 antibody or antigen binding fragment thereof to therapeutic polynucleotide that is at least about 100:1. In some embodiments, a pharmaceutical composition described herein has a molar ratio of 3E10 antibody or antigen binding fragment thereof to therapeutic polynucleotide that is at least about 125:1. In some embodiments, a pharmaceutical composition described herein has a molar ratio of 3E10 antibody or antigen binding fragment thereof to therapeutic polynucleotide that is at least about 150:1. In some embodiments, a pharmaceutical composition described herein has a molar ratio of 3E10 antibody or antigen binding fragment thereof to therapeutic polynucleotide that is at least about 200:1.

In some embodiments, a longer polynucleotide is at least 1000 nucleotides in length, e.g., 1000 nucleotides for a single-stranded polynucleotide or 1000 base pairs for a double-stranded polynucleotide. In some embodiments, a longer polynucleotide is at least 1500 nucleotides in length. In some embodiments, a longer polynucleotide is at least 2000 nucleotides in length. In some embodiments, a longer polynucleotide is at least 2500 nucleotides in length. In some embodiments, a longer polynucleotide is at least 3000 nucleotides in length. In some embodiments, a longer polynucleotide is at least 4000 nucleotides in length. In some embodiments, a longer polynucleotide is at least 5000 nucleotides in length. In some embodiments, a longer polynucleotide is at least 7500 nucleotides in length. In some embodiments, a longer polynucleotide is at least 10,000 nucleotides in length.

In some embodiments, a pharmaceutical composition described herein has a molar ratio of 3E10 antibody or antigen binding fragment thereof to therapeutic polynucleotide that is at least about 3:1, at least about 4:1, at least about 5:1, at least about 6:1, at least about 7:1, at least about 8:1, at least about 9:1, at least about 10:1, at least about 11:1, at least about 12:1, at least about 13:1, at least about 14:1, at least about 15:1, at least about 16:1, at least about 17:1, at least about 18:1, at least about 19:1, at least about 20:1, at least about 21:1, at least about 22:1, at least about 23:1, at least about 24:1, at least about 25:1, at least about 26:1, at least about 27:1, at least about 28:1, at least about 29:1, at least about 30:1, at least about 31:1, at least about 32:1, at least about 33:1, at least about 34:1, at least about 35:1, at least about 36:1, at least about 37:1, at least about 38:1, at least about 39:1, at least about 40:1, at least about 41:1, at least about 42:1, at least about 43:1, at least about 44:1, at least about 45:1, at least about 50:1, at least about 55:1, at least about 60:1, at least about 70:1, at least about 75:1, at least about 80:1, at least about 85:1, at least about 90:1, at least about 95:1, at least about 100:1, at least about 110:1, at least about 120:1, at least about 125:1, at least about 130:1, at least about 140:1, at least about 150:1, at least about 160:1, at least about 170:1, at least about 175:1, at least about 180:1, at least about 190:1, at least about 200:1, or greater.

In some embodiments, a pharmaceutical composition described herein has a molar ratio of 3E10 antibody or antigen binding fragment thereof to therapeutic polynucleotide that is at least 2:1. In some embodiments, a pharmaceutical composition described herein has a molar ratio of 3E10 antibody or antigen binding fragment thereof to therapeutic polynucleotide that is at least 5:1. In some embodiments, a pharmaceutical composition described herein has a molar ratio of 3E10 antibody or antigen binding fragment thereof to therapeutic polynucleotide that is at least 7.5:1. In some embodiments, a pharmaceutical composition described herein has a molar ratio of 3E10 antibody or antigen binding fragment thereof to therapeutic polynucleotide that is at least 10:1. In some embodiments, a pharmaceutical composition described herein has a molar ratio of 3E10 antibody or antigen binding fragment thereof to therapeutic polynucleotide that is at least 15:1. In some embodiments, a pharmaceutical composition described herein has a molar ratio of 3E10 antibody or antigen binding fragment thereof to therapeutic polynucleotide that is at least 20:1. In some embodiments, a pharmaceutical composition described herein has a molar ratio of 3E10 antibody or antigen binding fragment thereof to therapeutic polynucleotide that is at least 25:1. In some embodiments, a pharmaceutical composition described herein has a molar ratio of 3E10 antibody or antigen binding fragment thereof to therapeutic polynucleotide that is at least 30:1. In some embodiments, a pharmaceutical composition described herein has a molar ratio of 3E10 antibody or antigen binding fragment thereof to therapeutic polynucleotide that is at least 40:1. In some embodiments, a pharmaceutical composition described herein has a molar ratio of 3E10 antibody or antigen binding fragment thereof to therapeutic polynucleotide that is at least 50:1. In some embodiments, a pharmaceutical composition described herein has a molar ratio of 3E10 antibody or antigen binding fragment thereof to therapeutic polynucleotide that is at least 75:1. In some embodiments, a pharmaceutical composition described herein has a molar ratio of 3E10 antibody or antigen binding fragment thereof to therapeutic polynucleotide that is at least 100:1. In some embodiments, a pharmaceutical composition described herein has a molar ratio of 3E10 antibody or antigen binding fragment thereof to therapeutic polynucleotide that is at least 125:1. In some embodiments, a pharmaceutical composition described herein has a molar ratio of 3E10 antibody or antigen binding fragment thereof to therapeutic polynucleotide that is at least 150:1. In some embodiments, a pharmaceutical composition described herein has a molar ratio of 3E10 antibody or antigen binding fragment thereof to therapeutic polynucleotide that is at least 200:1.

In some embodiments, a pharmaceutical composition described herein has a molar ratio of 3E10 antibody or antigen binding fragment thereof to therapeutic polynucleotide that is at least 3:1, at least 4:1, at least 5:1, at least 6:1, at least 7:1, at least 8:1, at least 9:1, at least 10:1, at least 11:1, at least 12:1, at least 13:1, at least 14:1, at least 15:1, at least 16:1, at least 17:1, at least 18:1, at least 19:1, at least 20:1, at least 21:1, at least 22:1, at least 23:1, at least 24:1, at least 25:1, at least 26:1, at least 27:1, at least 28:1, at least 29:1, at least 30:1, at least 31:1, at least 32:1, at least 33:1, at least 34:1, at least 35:1, at least 36:1, at least 37:1, at least 38:1, at least 39:1, at least 40:1, at least 41:1, at least 42:1, at least 43:1, at least 44:1, at least 45:1, at least 50:1, at least 55:1, at least 60:1, at least 70:1, at least 75:1, at least 80:1, at least 85:1, at least 90:1, at least 95:1, at least 100:1, at least 110:1, at least 120:1, at least 125:1, at least 130:1, at least 140:1, at least 150:1, at least 160:1, at least 170:1, at least 175:1, at least 180:1, at least 190:1, at least 200:1, or greater.

In some embodiments, a pharmaceutical composition described herein has a molar ratio of 3E10 antibody or antigen binding fragment thereof to therapeutic polynucleotide that is 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 50:1, 55:1, 60:1, 70:1, 75:1, 80:1, 85:1, 90:1, 95:1, 100:1, 110:1, 120:1, 125:1, 130:1, 140:1, 150:1, 160:1, 170:1, 175:1, 180:1, 190:1, 200:1, or greater.

In some embodiments, a pharmaceutical composition described herein has a molar ratio of 3E10 antibody or antigen binding fragment thereof to therapeutic polynucleotide that is no more than about 200:1. In some embodiments, a pharmaceutical composition described herein has a molar ratio of 3E10 antibody or antigen binding fragment thereof to therapeutic polynucleotide that is no more than about 150:1. In some embodiments, a pharmaceutical composition described herein has a molar ratio of 3E10 antibody or antigen binding fragment thereof to therapeutic polynucleotide that is no more than about 100:1. In some embodiments, a pharmaceutical composition described herein has a molar ratio of 3E10 antibody or antigen binding fragment thereof to therapeutic polynucleotide that is no more than about 50:1. In some embodiments, a pharmaceutical composition described herein has a molar ratio of 3E10 antibody or antigen binding fragment thereof to therapeutic polynucleotide that is no more than about 40:1. In some embodiments, a pharmaceutical composition described herein has a molar ratio of 3E10 antibody or antigen binding fragment thereof to therapeutic polynucleotide that is no more than about 30:1. In some embodiments, a pharmaceutical composition described herein has a molar ratio of 3E10 antibody or antigen binding fragment thereof to therapeutic polynucleotide that is no more than about 25:1. In some embodiments, a pharmaceutical composition described herein has a molar ratio of 3E10 antibody or antigen binding fragment thereof to therapeutic polynucleotide that is no more than about 20:1. In some embodiments, a pharmaceutical composition described herein has a molar ratio of 3E10 antibody or antigen binding fragment thereof to therapeutic polynucleotide that is no more than about 15:1. In some embodiments, a pharmaceutical composition described herein has a molar ratio of 3E10 antibody or antigen binding fragment thereof to therapeutic polynucleotide that is no more than about 10:1.

In some embodiments, a pharmaceutical composition described herein has a molar ratio of 3E10 antibody or antigen binding fragment thereof to therapeutic polynucleotide that is no more than about 200:1, no more than about 175:1, no more than about 150:1, no more than about 125:1, no more than about 100:1, no more than about 75:1, no more than about 50:1, no more than about 45:1, no more than about 40:1, no more than about 35:1, no more than about 30:1, no more than about 35:1, no more than about 30:1, no more than about 25:1, no more than about 20:1, or less.

In some embodiments, a pharmaceutical composition described herein has a molar ratio of 3E10 antibody or antigen binding fragment thereof to therapeutic polynucleotide that is no more than 200:1. In some embodiments, a pharmaceutical composition described herein has a molar ratio of 3E10 antibody or antigen binding fragment thereof to therapeutic polynucleotide that is no more than 150:1. In some embodiments, a pharmaceutical composition described herein has a molar ratio of 3E10 antibody or antigen binding fragment thereof to therapeutic polynucleotide that is no more than 100:1. In some embodiments, a pharmaceutical composition described herein has a molar ratio of 3E10 antibody or antigen binding fragment thereof to therapeutic polynucleotide that is no more than 50:1. In some embodiments, a pharmaceutical composition described herein has a molar ratio of 3E10 antibody or antigen binding fragment thereof to therapeutic polynucleotide that is no more than 40:1. In some embodiments, a pharmaceutical composition described herein has a molar ratio of 3E10 antibody or antigen binding fragment thereof to therapeutic polynucleotide that is no more than 30:1. In some embodiments, a pharmaceutical composition described herein has a molar ratio of 3E10 antibody or antigen binding fragment thereof to therapeutic polynucleotide that is no more than 25:1. In some embodiments, a pharmaceutical composition described herein has a molar ratio of 3E10 antibody or antigen binding fragment thereof to therapeutic polynucleotide that is no more than 20:1. In some embodiments, a pharmaceutical composition described herein has a molar ratio of 3E10 antibody or antigen binding fragment thereof to therapeutic polynucleotide that is no more than 15:1. In some embodiments, a pharmaceutical composition described herein has a molar ratio of 3E10 antibody or antigen binding fragment thereof to therapeutic polynucleotide that is no more than 10:1.

In some embodiments, a pharmaceutical composition described herein has a molar ratio of 3E10 antibody or antigen binding fragment thereof to therapeutic polynucleotide that is no more than 200:1, no more than 175:1, no more than 150:1, no more than 125:1, no more than 100:1, no more than 75:1, no more than 50:1, no more than 45:1, no more than 40:1, no more than 35:1, no more than 30:1, no more than 35:1, no more than 30:1, no more than 25:1, no more than 20:1, or less.

In some embodiments, a pharmaceutical composition described herein has a molar ratio of 3E10 antibody or antigen binding fragment thereof to therapeutic polynucleotide that is of from 2:1 to 200:1. In some embodiments, a pharmaceutical composition described herein has a molar ratio of 3E10 antibody or antigen binding fragment thereof to therapeutic polynucleotide that is of from 2:1 to 175:1. In some embodiments, a pharmaceutical composition described herein has a molar ratio of 3E10 antibody or antigen binding fragment thereof to therapeutic polynucleotide that is of from 2:1 to 150:1. In some embodiments, a pharmaceutical composition described herein has a molar ratio of 3E10 antibody or antigen binding fragment thereof to therapeutic polynucleotide that is of from 2:1 to 125:1. In some embodiments, a pharmaceutical composition described herein has a molar ratio of 3E10 antibody or antigen binding fragment thereof to therapeutic polynucleotide that is of from 2:1 to 100:1. In some embodiments, a pharmaceutical composition described herein has a molar ratio of 3E10 antibody or antigen binding fragment thereof to therapeutic polynucleotide that is of from 2:1 to 75:1. In some embodiments, a pharmaceutical composition described herein has a molar ratio of 3E10 antibody or antigen binding fragment thereof to therapeutic polynucleotide that is of from 2:1 to 50:1. In some embodiments, a pharmaceutical composition described herein has a molar ratio of 3E10 antibody or antigen binding fragment thereof to therapeutic polynucleotide that is of from 2:1 to 40:1. In some embodiments, a pharmaceutical composition described herein has a molar ratio of 3E10 antibody or antigen binding fragment thereof to therapeutic polynucleotide that is of from 2:1 to 30:1. In some embodiments, a pharmaceutical composition described herein has a molar ratio of 3E10 antibody or antigen binding fragment thereof to therapeutic polynucleotide that is of from 2:1 to 25:1. In some embodiments, a pharmaceutical composition described herein has a molar ratio of 3E10 antibody or antigen binding fragment thereof to therapeutic polynucleotide that is of from 2:1 to 20:1. In some embodiments, a pharmaceutical composition described herein has a molar ratio of 3E10 antibody or antigen binding fragment thereof to therapeutic polynucleotide that is of from 2:1 to 15:1. In some embodiments, a pharmaceutical composition described herein has a molar ratio of 3E10 antibody or antigen binding fragment thereof to therapeutic polynucleotide that is of from 2:1 to 10:1. In some embodiments, a pharmaceutical composition described herein has a molar ratio of 3E10 antibody or antigen binding fragment thereof to therapeutic polynucleotide that is of from 2:1 to 7.5:1. In some embodiments, a pharmaceutical composition described herein has a molar ratio of 3E10 antibody or antigen binding fragment thereof to therapeutic polynucleotide that is of from 2:1 to 5:1. In some embodiments, a pharmaceutical composition described herein has a molar ratio of 3E10 antibody or antigen binding fragment thereof to therapeutic polynucleotide that is of from 2:1 to 3:1.

In some embodiments, a pharmaceutical composition described herein has a molar ratio of 3E10 antibody or antigen binding fragment thereof to therapeutic polynucleotide that is of from 3:1 to 200:1. In some embodiments, a pharmaceutical composition described herein has a molar ratio of 3E10 antibody or antigen binding fragment thereof to therapeutic polynucleotide that is of from 3:1 to 175:1. In some embodiments, a pharmaceutical composition described herein has a molar ratio of 3E10 antibody or antigen binding fragment thereof to therapeutic polynucleotide that is of from 3:1 to 150:1. In some embodiments, a pharmaceutical composition described herein has a molar ratio of 3E10 antibody or antigen binding fragment thereof to therapeutic polynucleotide that is of from 3:1 to 125:1. In some embodiments, a pharmaceutical composition described herein has a molar ratio of 3E10 antibody or antigen binding fragment thereof to therapeutic polynucleotide that is of from 3:1 to 100:1. In some embodiments, a pharmaceutical composition described herein has a molar ratio of 3E10 antibody or antigen binding fragment thereof to therapeutic polynucleotide that is of from 3:1 to 75:1. In some embodiments, a pharmaceutical composition described herein has a molar ratio of 3E10 antibody or antigen binding fragment thereof to therapeutic polynucleotide that is of from 3:1 to 50:1. In some embodiments, a pharmaceutical composition described herein has a molar ratio of 3E10 antibody or antigen binding fragment thereof to therapeutic polynucleotide that is of from 3:1 to 40:1. In some embodiments, a pharmaceutical composition described herein has a molar ratio of 3E10 antibody or antigen binding fragment thereof to therapeutic polynucleotide that is of from 3:1 to 30:1. In some embodiments, a pharmaceutical composition described herein has a molar ratio of 3E10 antibody or antigen binding fragment thereof to therapeutic polynucleotide that is of from 3:1 to 25:1. In some embodiments, a pharmaceutical composition described herein has a molar ratio of 3E10 antibody or antigen binding fragment thereof to therapeutic polynucleotide that is of from 3:1 to 20:1. In some embodiments, a pharmaceutical composition described herein has a molar ratio of 3E10 antibody or antigen binding fragment thereof to therapeutic polynucleotide that is of from 3:1 to 15:1. In some embodiments, a pharmaceutical composition described herein has a molar ratio of 3E10 antibody or antigen binding fragment thereof to therapeutic polynucleotide that is of from 3:1 to 10:1. In some embodiments, a pharmaceutical composition described herein has a molar ratio of 3E10 antibody or antigen binding fragment thereof to therapeutic polynucleotide that is of from 3:1 to 7.5:1. In some embodiments, a pharmaceutical composition described herein has a molar ratio of 3E10 antibody or antigen binding fragment thereof to therapeutic polynucleotide that is of from 3:1 to 5:1.

In some embodiments, a pharmaceutical composition described herein has a molar ratio of 3E10 antibody or antigen binding fragment thereof to therapeutic polynucleotide that is of from 5:1 to 200:1. In some embodiments, a pharmaceutical composition described herein has a molar ratio of 3E10 antibody or antigen binding fragment thereof to therapeutic polynucleotide that is of from 5:1 to 175:1. In some embodiments, a pharmaceutical composition described herein has a molar ratio of 3E10 antibody or antigen binding fragment thereof to therapeutic polynucleotide that is of from 5:1 to 150:1. In some embodiments, a pharmaceutical composition described herein has a molar ratio of 3E10 antibody or antigen binding fragment thereof to therapeutic polynucleotide that is of from 5:1 to 125:1. In some embodiments, a pharmaceutical composition described herein has a molar ratio of 3E10 antibody or antigen binding fragment thereof to therapeutic polynucleotide that is of from 5:1 to 100:1. In some embodiments, a pharmaceutical composition described herein has a molar ratio of 3E10 antibody or antigen binding fragment thereof to therapeutic polynucleotide that is of from 5:1 to 75:1. In some embodiments, a pharmaceutical composition described herein has a molar ratio of 3E10 antibody or antigen binding fragment thereof to therapeutic polynucleotide that is of from 5:1 to 50:1. In some embodiments, a pharmaceutical composition described herein has a molar ratio of 3E10 antibody or antigen binding fragment thereof to therapeutic polynucleotide that is of from 5:1 to 40:1. In some embodiments, a pharmaceutical composition described herein has a molar ratio of 3E10 antibody or antigen binding fragment thereof to therapeutic polynucleotide that is of from 5:1 to 30:1. In some embodiments, a pharmaceutical composition described herein has a molar ratio of 3F10 antibody or antigen binding fragment thereof to therapeutic polynucleotide that is of from 5:1 to 25:1. In some embodiments, a pharmaceutical composition described herein has a molar ratio of 3E10 antibody or antigen binding fragment thereof to therapeutic polynucleotide that is of from 5:1 to 20:1. In some embodiments, a pharmaceutical composition described herein has a molar ratio of 3E10 antibody or antigen binding fragment thereof to therapeutic polynucleotide that is of from 5:1 to 15:1. In some embodiments, a pharmaceutical composition described herein has a molar ratio of 3E10 antibody or antigen binding fragment thereof to therapeutic polynucleotide that is of from 5:1 to 10:1. In some embodiments, a pharmaceutical composition described herein has a molar ratio of 3E10 antibody or antigen binding fragment thereof to therapeutic polynucleotide that is of from 5:1 to 7.5:1.

In some embodiments, a pharmaceutical composition described herein has a molar ratio of 3E10 antibody or antigen binding fragment thereof to therapeutic polynucleotide that is of from 7.5:1 to 200:1. In some embodiments, a pharmaceutical composition described herein has a molar ratio of 3E10 antibody or antigen binding fragment thereof to therapeutic polynucleotide that is of from 7.5:1 to 175:1. In some embodiments, a pharmaceutical composition described herein has a molar ratio of 3E10 antibody or antigen binding fragment thereof to therapeutic polynucleotide that is of from 7.5:1 to 150:1. In some embodiments, a pharmaceutical composition described herein has a molar ratio of 3E10 antibody or antigen binding fragment thereof to therapeutic polynucleotide that is of from 7.5:1 to 125:1. In some embodiments, a pharmaceutical composition described herein has a molar ratio of 3E10 antibody or antigen binding fragment thereof to therapeutic polynucleotide that is of from 7.5:1 to 100:1. In some embodiments, a pharmaceutical composition described herein has a molar ratio of 3E10 antibody or antigen binding fragment thereof to therapeutic polynucleotide that is of from 7.5:1 to 75:1. In some embodiments, a pharmaceutical composition described herein has a molar ratio of 3E10 antibody or antigen binding fragment thereof to therapeutic polynucleotide that is of from 7.5:1 to 50:1. In some embodiments, a pharmaceutical composition described herein has a molar ratio of 3E10 antibody or antigen binding fragment thereof to therapeutic polynucleotide that is of from 7.5:1 to 40:1. In some embodiments, a pharmaceutical composition described herein has a molar ratio of 3E10 antibody or antigen binding fragment thereof to therapeutic polynucleotide that is of from 7.5:1 to 30:1. In some embodiments, a pharmaceutical composition described herein has a molar ratio of 3E10 antibody or antigen binding fragment thereof to therapeutic polynucleotide that is of from 7.5:1 to 25:1. In some embodiments, a pharmaceutical composition described herein has a molar ratio of 3E10 antibody or antigen binding fragment thereof to therapeutic polynucleotide that is of from 7.5:1 to 20:1. In some embodiments, a pharmaceutical composition described herein has a molar ratio of 3E10 antibody or antigen binding fragment thereof to therapeutic polynucleotide that is of from 7.5:1 to 15:1. In some embodiments, a pharmaceutical composition described herein has a molar ratio of 3E10 antibody or antigen binding fragment thereof to therapeutic polynucleotide that is of from 7.5:1 to 10:1.

In some embodiments, a pharmaceutical composition described herein has a molar ratio of 3E10 antibody or antigen binding fragment thereof to therapeutic polynucleotide that is of from 10:1 to 200:1. In some embodiments, a pharmaceutical composition described herein has a molar ratio of 3E10 antibody or antigen binding fragment thereof to therapeutic polynucleotide that is of from 10:1 to 175:1. In some embodiments, a pharmaceutical composition described herein has a molar ratio of 3E10 antibody or antigen binding fragment thereof to therapeutic polynucleotide that is of from 10:1 to 150:1. In some embodiments, a pharmaceutical composition described herein has a molar ratio of 3E10 antibody or antigen binding fragment thereof to therapeutic polynucleotide that is of from 10:1 to 125:1. In some embodiments, a pharmaceutical composition described herein has a molar ratio of 3E10 antibody or antigen binding fragment thereof to therapeutic polynucleotide that is of from 10:1 to 100:1. In some embodiments, a pharmaceutical composition described herein has a molar ratio of 3E10 antibody or antigen binding fragment thereof to therapeutic polynucleotide that is of from 10:1 to 75:1. In some embodiments, a pharmaceutical composition described herein has a molar ratio of 3E10 antibody or antigen binding fragment thereof to therapeutic polynucleotide that is of from 10:1 to 50:1. In some embodiments, a pharmaceutical composition described herein has a molar ratio of 3E10 antibody or antigen binding fragment thereof to therapeutic polynucleotide that is of from 10:1 to 40:1. In some embodiments, a pharmaceutical composition described herein has a molar ratio of 3E10 antibody or antigen binding fragment thereof to therapeutic polynucleotide that is of from 10:1 to 30:1. In some embodiments, a pharmaceutical composition described herein has a molar ratio of 3F10 antibody or antigen binding fragment thereof to therapeutic polynucleotide that is of from 10:1 to 25:1. In some embodiments, a pharmaceutical composition described herein has a molar ratio of 3E10 antibody or antigen binding fragment thereof to therapeutic polynucleotide that is of from 10:1 to 20:1. In some embodiments, a pharmaceutical composition described herein has a molar ratio of 3E10 antibody or antigen binding fragment thereof to therapeutic polynucleotide that is of from 10:1 to 15:1.

In some embodiments, a pharmaceutical composition described herein has a molar ratio of 3E10 antibody or antigen binding fragment thereof to therapeutic polynucleotide that is of from 15:1 to 200:1. In some embodiments, a pharmaceutical composition described herein has a molar ratio of 3E10 antibody or antigen binding fragment thereof to therapeutic polynucleotide that is of from 15:1 to 175:1. In some embodiments, a pharmaceutical composition described herein has a molar ratio of 3E10 antibody or antigen binding fragment thereof to therapeutic polynucleotide that is of from 15:1 to 150:1. In some embodiments, a pharmaceutical composition described herein has a molar ratio of 3E10 antibody or antigen binding fragment thereof to therapeutic polynucleotide that is of from 15:1 to 125:1. In some embodiments, a pharmaceutical composition described herein has a molar ratio of 3E10 antibody or antigen binding fragment thereof to therapeutic polynucleotide that is of from 15:1 to 100:1. In some embodiments, a pharmaceutical composition described herein has a molar ratio of 3E10 antibody or antigen binding fragment thereof to therapeutic polynucleotide that is of from 15:1 to 75:1. In some embodiments, a pharmaceutical composition described herein has a molar ratio of 3E10 antibody or antigen binding fragment thereof to therapeutic polynucleotide that is of from 15:1 to 50:1. In some embodiments, a pharmaceutical composition described herein has a molar ratio of 3E10 antibody or antigen binding fragment thereof to therapeutic polynucleotide that is of from 15:1 to 40:1. In some embodiments, a pharmaceutical composition described herein has a molar ratio of 3E10 antibody or antigen binding fragment thereof to therapeutic polynucleotide that is of from 15:1 to 30:1. In some embodiments, a pharmaceutical composition described herein has a molar ratio of 3E10 antibody or antigen binding fragment thereof to therapeutic polynucleotide that is of from 15:1 to 25:1. In some embodiments, a pharmaceutical composition described herein has a molar ratio of 3E10 antibody or antigen binding fragment thereof to therapeutic polynucleotide that is of from 15:1 to 20:1.

In some embodiments, a pharmaceutical composition described herein has a molar ratio of 3E10 antibody or antigen binding fragment thereof to therapeutic polynucleotide that is of from 20:1 to 200:1. In some embodiments, a pharmaceutical composition described herein has a molar ratio of 3E10 antibody or antigen binding fragment thereof to therapeutic polynucleotide that is of from 20:1 to 175:1. In some embodiments, a pharmaceutical composition described herein has a molar ratio of 3E10 antibody or antigen binding fragment thereof to therapeutic polynucleotide that is of from 20:1 to 150:1. In some embodiments, a pharmaceutical composition described herein has a molar ratio of 3E10 antibody or antigen binding fragment thereof to therapeutic polynucleotide that is of from 20:1 to 125:1. In some embodiments, a pharmaceutical composition described herein has a molar ratio of 3E10 antibody or antigen binding fragment thereof to therapeutic polynucleotide that is of from 20:1 to 100:1. In some embodiments, a pharmaceutical composition described herein has a molar ratio of 3E10 antibody or antigen binding fragment thereof to therapeutic polynucleotide that is of from 20:1 to 75:1. In some embodiments, a pharmaceutical composition described herein has a molar ratio of 3E10 antibody or antigen binding fragment thereof to therapeutic polynucleotide that is of from 20:1 to 50:1. In some embodiments, a pharmaceutical composition described herein has a molar ratio of 3E10 antibody or antigen binding fragment thereof to therapeutic polynucleotide that is of from 20:1 to 40:1. In some embodiments, a pharmaceutical composition described herein has a molar ratio of 3E10 antibody or antigen binding fragment thereof to therapeutic polynucleotide that is of from 20:1 to 30:1. In some embodiments, a pharmaceutical composition described herein has a molar ratio of 3E10 antibody or antigen binding fragment thereof to therapeutic polynucleotide that is of from 20:1 to 25:1.

In some embodiments, a pharmaceutical composition described herein has a molar ratio of 3E10 antibody or antigen binding fragment thereof to therapeutic polynucleotide that is of from 25:1 to 200:1. In some embodiments, a pharmaceutical composition described herein has a molar ratio of 3E10 antibody or antigen binding fragment thereof to therapeutic polynucleotide that is of from 25:1 to 175:1. In some embodiments, a pharmaceutical composition described herein has a molar ratio of 3E10 antibody or antigen binding fragment thereof to therapeutic polynucleotide that is of from 25:1 to 150:1. In some embodiments, a pharmaceutical composition described herein has a molar ratio of 3E10 antibody or antigen binding fragment thereof to therapeutic polynucleotide that is of from 25:1 to 125:1. In some embodiments, a pharmaceutical composition described herein has a molar ratio of 3E10 antibody or antigen binding fragment thereof to therapeutic polynucleotide that is of from 25:1 to 100:1. In some embodiments, a pharmaceutical composition described herein has a molar ratio of 3E10 antibody or antigen binding fragment thereof to therapeutic polynucleotide that is of from 25:1 to 75:1. In some embodiments, a pharmaceutical composition described herein has a molar ratio of 3E10 antibody or antigen binding fragment thereof to therapeutic polynucleotide that is of from 25:1 to 50:1. In some embodiments, a pharmaceutical composition described herein has a molar ratio of 3E10 antibody or antigen binding fragment thereof to therapeutic polynucleotide that is of from 25:1 to 40:1. In some embodiments, a pharmaceutical composition described herein has a molar ratio of 3E10 antibody or antigen binding fragment thereof to therapeutic polynucleotide that is of from 25:1 to 30:1.

In yet other embodiments, other ranges falling with the range of about 2:1 to about 200:1 are contemplated.

In some embodiments, a pharmaceutical composition described herein has a molar ratio of 3E10 antibody or antigen binding fragment thereof to therapeutic polynucleotide that is of from about 1:1 to about 200:1. In some embodiments, a pharmaceutical composition described herein has a molar ratio of 3E10 antibody or antigen binding fragment thereof to therapeutic polynucleotide that is of from about 1:1 to about 175:1. In some embodiments, a pharmaceutical composition described herein has a molar ratio of 3E10 antibody or antigen binding fragment thereof to therapeutic polynucleotide that is of from about 1:1 to about 150:1. In some embodiments, a pharmaceutical composition described herein has a molar ratio of 3E10 antibody or antigen binding fragment thereof to therapeutic polynucleotide that is of from about 1:1 to about 125:1. In some embodiments, a pharmaceutical composition described herein has a molar ratio of 3E10 antibody or antigen binding fragment thereof to therapeutic polynucleotide that is of from about 1:1 to about 100:1. In some embodiments, a pharmaceutical composition described herein has a molar ratio of 3E10 antibody or antigen binding fragment thereof to therapeutic polynucleotide that is of from about 1:1 to about 75:1. In some embodiments, a pharmaceutical composition described herein has a molar ratio of 3E10 antibody or antigen binding fragment thereof to therapeutic polynucleotide that is of from about 1:1 to about 50:1. In some embodiments, a pharmaceutical composition described herein has a molar ratio of 3E10 antibody or antigen binding fragment thereof to therapeutic polynucleotide that is of from about 1:1 to about 30:1. In some embodiments, a pharmaceutical composition described herein has a molar ratio of 3E10 antibody or antigen binding fragment thereof to therapeutic polynucleotide that is of from about 1:1 to about 20:1. In some embodiments, a pharmaceutical composition described herein has a molar ratio of 3E10 antibody or variant thereof, or antigen-binding fragment thereof to therapeutic polynucleotide that is of from about 1:1 to about 10:1. In some embodiments, a pharmaceutical composition described herein has a molar ratio of 3E10 antibody or variant thereof, or antigen-binding fragment thereof to therapeutic polynucleotide that is of from about 1:1 to about 5:1.

In some embodiments, a pharmaceutical composition described herein has a molar ratio of 3E10 antibody or antigen binding fragment thereof to therapeutic polynucleotide that is at least about 1:1, at least about 1:2, at least about 1:3, at least about 1:4, at least about 1:5, at least about 1:6, at least about 1:7, at least about 1:8, at least about 1:9, at least about 1:10, at least about 1:11, at least about 1:12, at least about 1:13, at least about 1:14, at least about 1:15, at least about 1:16, at least about 1:17, at least about 1:18, at least about 1:19, at least about 1:20, at least about 1:21, at least about 1:22, at least about 1:23, at least about 1:24, at least about 1:25, at least about 1:26, at least about 1:27, at least about 1:28, at least about 1:29, at least about 1:30, or greater.

In some embodiments, a pharmaceutical composition described herein has a molar ratio of 3E10 antibody or antigen binding fragment thereof to therapeutic polynucleotide that is at least 1:1. In some embodiments, a pharmaceutical composition described herein has a molar ratio of 3E10 antibody or antigen binding fragment thereof to therapeutic polynucleotide that is at least 1:2. In some embodiments, a pharmaceutical composition described herein has a molar ratio of 3F10 antibody or antigen binding fragment thereof to therapeutic polynucleotide that is at least 1:3. In some embodiments, a pharmaceutical composition described herein has a molar ratio of 3E10 antibody or antigen binding fragment thereof to therapeutic polynucleotide that is at least 1:4. In some embodiments, a pharmaceutical composition described herein has a molar ratio of 3E10 antibody or antigen binding fragment thereof to therapeutic polynucleotide that is at least 1:5. In some embodiments, a pharmaceutical composition described herein has a molar ratio of 3E10 antibody or antigen binding fragment thereof to therapeutic polynucleotide that is at least 1:10. In some embodiments, a pharmaceutical composition described herein has a molar ratio of 3E10 antibody or antigen binding fragment thereof to therapeutic polynucleotide that is at least 1:15. In some embodiments, a pharmaceutical composition described herein has a molar ratio of 3E10 antibody or antigen binding fragment thereof to therapeutic polynucleotide that is at least 1:20. In some embodiments, a pharmaceutical composition described herein has a molar ratio of 3E10 antibody or antigen binding fragment thereof to therapeutic polynucleotide that is at least 1:25. In some embodiments, a pharmaceutical composition described herein has a molar ratio of 3E10 antibody or antigen binding fragment thereof to therapeutic polynucleotide that is at least 1:30.

In some embodiments, a pharmaceutical composition described herein has a molar ratio of 3E10 antibody or antigen binding fragment thereof to therapeutic polynucleotide that is at least 1:1, at least 1:2, at least 1:3, at least 1:4, at least 1:5, at least 1:6, at least 1:7, at least 1:8, at least 1:9, at least 1:10, at least 1:11, at least 1:12, at least 1:13, at least 1:14, at least 1:15, at least 1:16, at least 1:17, at least 1:18, at least 1:19, at least 1:20, at least 1:21, at least 1:22, at least 1:23, at least 1:24, at least 1:25, at least 1:26, at least 1:27, at least 1:28, at least 1:29, at least 1:30, or greater.

In some embodiments, a pharmaceutical composition described herein has a molar ratio of 3E10 antibody or antigen binding fragment thereof to therapeutic polynucleotide that is 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, or greater

The 3E10 antibodies or variants thereof, or antigen-binding fragments thereof as disclosed herein, can localize to cardiac muscle tissue in vivo following systemic administration. Accordingly, the compositions described herein are well suited for the delivery of therapeutic mRNAs encoding proteins useful for treating disorders of cardiac muscle tissue. Accordingly, in some embodiments, the therapeutic mRNA polynucleotide encodes a cardiac muscle polypeptide.

The 3E10 antibodies or variants thereof, or antigen-binding fragments thereof as disclosed herein, can localize to hepatic tissue in vivo following systemic administration. Accordingly, the compositions described herein are well suited for the delivery of therapeutic mRNAs encoding proteins useful for treating disorders of hepatic tissue. Accordingly, in some embodiments, the therapeutic mRNA polynucleotide encodes a hepatic polypeptide.

The 3E10 antibodies or variants thereof, or antigen-binding fragments thereof as disclosed herein, can localize to lung tissue (e.g., epithelium) in vivo following systemic administration. Accordingly, the compositions described herein are well suited for the delivery of therapeutic mRNAs encoding proteins useful for treating disorders of lung tissue. Accordingly, in some embodiments, the therapeutic mRNA polynucleotide encodes a lung tissue polypeptide.

The 3E10 antibodies or variants thereof, or antigen-binding fragments thereof as disclosed herein, can localize to neuronal tissue in vivo following systemic administration. Accordingly, the compositions described herein are well suited for the delivery of therapeutic mRNAs encoding proteins useful for treating disorders of neuronal tissue. Accordingly, in some embodiments, the therapeutic mRNA polynucleotide encodes a neuronal polypeptide.

Examples of proteins, and their associated genes, are disclosed herein. Generally, any one of these proteins, and variants thereof retaining a function of the full-length protein, can be encoded by the therapeutic mRNAs disclosed herein.

Moreover, because 3E10 antibodies or variants thereof, or antigen-binding fragments thereof localize to cardiac muscle tissue in vivo following systemic administration, the compositions of the present disclosure can be formulated for, and subsequently administered by, one of many common administrative routes. In some embodiments, the pharmaceutical composition is formulated for parenteral administration. In some embodiments, the parenteral administration is intramuscular administration, intravenous administration, or subcutaneous administration.

Moreover, because 3E10 antibodies or variants thereof, or antigen-binding fragments thereof localize to hepatic tissue in vivo following systemic administration, the compositions of the present disclosure can be formulated for, and subsequently administered by, one of many common administrative routes. In some embodiments, the pharmaceutical composition is formulated for parenteral administration. In some embodiments, the parenteral administration is intramuscular administration, intravenous administration, or subcutaneous administration.

Moreover, because 3E10 antibodies or variants thereof, or antigen-binding fragments thereof localize to lung tissue in vivo following systemic administration, the compositions of the present disclosure can be formulated for, and subsequently administered by, one of many common administrative routes. In some embodiments, the pharmaceutical composition is formulated for parenteral administration. In some embodiments, the parenteral administration is intramuscular administration, intravenous administration, or subcutaneous administration.

Moreover, because 3E10 antibodies or variants thereof, or antigen-binding fragments thereof localize to neuronal tissue in vivo following systemic administration, the compositions of the present disclosure can be formulated for, and subsequently administered by, one of many common administrative routes. In some embodiments, the pharmaceutical composition is formulated for parenteral administration. In some embodiments, the parenteral administration is intramuscular administration, intravenous administration, or subcutaneous administration.

In some embodiments, the mRNA of the compositions described herein are codon-optimized, e.g., to improve half-life or increase translation in cardiac muscle tissue.

In some embodiments, the mRNA of the compositions described herein are codon-optimized, e.g., to improve half-life or increase translation in hepatic tissue.

In some embodiments, the mRNA of the compositions described herein are codon-optimized, e.g., to improve half-life or increase translation in lung tissue.

In some embodiments, the mRNA of the compositions described herein are codon-optimized, e.g., to improve half-life r increase translation in neuronal tissue.

Codon-optimized refers to a polynucleotide sequence encoding a polypeptide (e.g., a cardiac muscle polypeptide, neuronal polypeptide), where at least one codon of the native polynucleotide encoding the polypeptide has been changed to improve a property of the polynucleotide sequence. In some embodiments, the improved property promotes increased transcription of mRNA coding for the polypeptide, increased stability of the mRNA (e.g., improved mRNA half-life), increased translation of the polypeptide, and/or increased packaging of the polynucleotide within the vector. Non-limiting examples of alterations that can be used to achieve the improved properties include changing the usage and/or distribution of codons for particular amino acids, adjusting global and/or local GC content, removing AT-rich sequences, removing repeated sequence elements, adjusting global and/or local CpG dinucleotide content, removing cryptic regulatory elements (e.g., TATA box and CCAAT box elements), removing of intron/exon splice sites, improving regulatory sequences (e.g., introduction of a Kozak consensus sequence), and removing sequence elements capable of forming secondary structure (e.g., stem-loops) in the transcribed mRNA.

Similarly, in some embodiments, the mRNA of the compositions described herein include one or more non-canonical nucleotides, e.g., to improve the stability and/or half-life of the mRNA in vivo. Examples of non-canonical nucleotides suitable for inclusion in the mRNA molecules described herein are described in U.S. Pat. No. 9,181,319, the content of which is incorporated herein by reference.

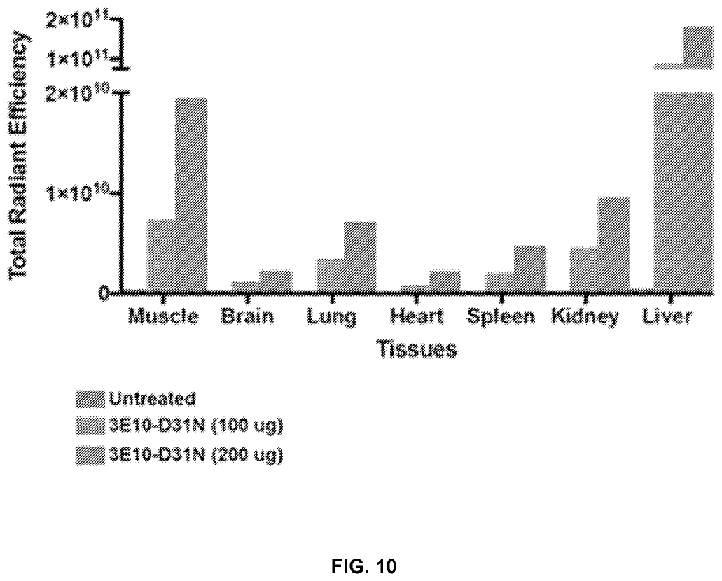

It was observed that tissue localization following intravenous administration of 3E10 in mice was dose dependent. Specifically, as shown in FIG. 10, a roughly two-fold increase in 3E10 accumulation occurred in several tissues when dosing of the composition doubled. Further, 3E10 accumulated in different tissues at different levels. For instance, as shown in FIG. 10, 3E10 accumulated in liver tissue at 1 to 2 orders of magnitude more than in other tissues. Similarly, 3E10 accumulated in muscle tissue at about an order of magnitude more than in tissues such as brain, lung, heart, spleen, and kidney. Similarly, 3E10 appeared to accumulate in lung and kidney tissue at higher levels than in brain and heart tissue.

Accordingly, in some embodiments, dosing of the 3E10-mRNA compositions described herein will be dependent upon the tissue being targeted. For example, in some embodiments, dosing will be higher when delivering an mRNA to brain or heart tissue than when delivering to muscle, lung, spleen, kidney, or liver tissue. Similarly, in some embodiments, dosing will be higher when delivering an mRNA to lung, spleen, or kidney tissue than when delivering to muscle or liver tissue. Similarly, in some embodiments, dosing will be higher when delivering an mRNA to muscle tissue than when delivering to liver tissue. Other factors, for example, the translational efficiency for a particular mRNA construct can also be considered when determining dosing for a particular composition.

In some aspects, the disclosure encompasses kits comprising one or more containers and comprising one or more doses of a complex formed between a therapeutic mRNA molecule and a 3E10 antibody or antigen binding fragment thereof disclosed herein. In certain embodiments, a unit dosage is provided wherein the unit dosage contains a predetermined amount of a composition comprising, for example, a complex formed between a therapeutic mRNA molecule and a 3E10 antibody or antigen binding fragment thereof disclosed herein, with or without one or more additional agents. For other embodiments, such a unit dosage is supplied in single-use prefilled syringe for injection. In still other embodiments, the composition contained in the unit dosage may comprise saline, sucrose, or the like; a buffer, such as phosphate, or the like; and/or be formulated within a stable and effective pH range. Alternatively, in certain embodiments, the composition may be provided as a lyophilized powder that may be reconstituted upon addition of an appropriate liquid, for example, sterile water. In certain preferred embodiments, the composition comprises one or more substances that inhibit protein aggregation, including, but not limited to, sucrose and arginine. Any label on, or associated with, the container(s) indicates that the enclosed composition is used for diagnosis or treatment.

The present invention also provides kits for producing single-dose or multi-dose administration units of a complex formed between a therapeutic mRNA molecule and a 3E10 antibody or antigen binding fragment thereof disclosed herein and, optionally, one or more other diagnostic or therapeutic agents. The kit comprises a container and a label or package insert on or associated with the container. Suitable containers include, for example, bottles, vials, syringes, etc. The containers may be formed from a variety of materials such as glass or plastic. The container holds a composition that is effective for treating the condition and may have a sterile access port (for example the container may be an intravenous solution bag or a vial having a stopper pierceable by a hypodermic injection needle). Such kits will generally contain a pharmaceutically acceptable formulation of a complex formed between a therapeutic mRNA molecule and a 3E10 antibody or antigen binding fragment thereof and, optionally, one or more other therapeutic agents in the same or different suitable containers. The kits may also contain other pharmaceutically acceptable formulations, for combination therapy.

More specifically the kits may have a single container that contains the complex formed between a therapeutic mRNA molecule and a 3E10 antibody or antigen binding fragment thereof), with or without additional components, or they may have distinct containers for each desired agent. Alternatively, the complex formed between a therapeutic mRNA molecule and a 3E10 antibody or antigen binding fragment thereof and any optional therapeutic agent of the kit may be maintained separately within distinct containers prior to administration to a patient. The kits may also comprise a second/third container means for containing a sterile, pharmaceutically acceptable buffer or other diluent such as bacteriostatic water for injection (BWFI), phosphate-buffered saline (PBS), Ringer's solution and dextrose solution.

When the components of the kit are provided in one or more liquid solutions, the liquid solution is preferably an aqueous solution, with a sterile aqueous solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

As indicated briefly above the kits may also contain a means by which to administer the complex formed between a therapeutic mRNA molecule and a 3E10 antibody or antigen binding fragment thereof and any optional components to the patient, e.g., one or more needles or syringes, from which the formulation may be injected or introduced into the patient.

### EXAMPLES

With respect to the experiments below, standard 3E10 sequence was used except wherein noted to be the D3IN variant (e.g., Example 2). Both standard 3E10 and the D3IN variant were used as full length antibodies.

### Example 1—Carrier DNA Enhances mRNA to Non-Tumor Tissue

2 ug of fluorescently labeled mRNA was mixed with 20 ug of 3E10-D3IN with or without carrier DNA (5 ug) for 15 minutes at room temperature. mRNA complexed to 3E10 was injected into C57BL/6 murine fetuses at E15.5. At 24-48 hours after treatment, C57BL/6 murine fetuses were harvested and analyzed for mRNA delivery using IVIS imaging.

Without carrier DNA, the 3E10-D3IN complexed to mRNA was rapidly cleared from C57BL/6 murine fetuses at 24 hours. The addition of carrier DNA, however, resulted in detectable mRNA signal in multiple tissues (not harvested) of the C57BL/6 murine fetus at 48 hours (FIG. 19).

### Example 2—Molecular Modeling of 3E10 and Engineered Variants Thereof

WT HEAVY CHAIN scFv SEQUENCE
(SEQ ID NO: XX)
E VQLVESGGGL VKPGGSRKLS CAASGFTFSD YGMHWVRQAP
EKGLEWVAYI SSGSSTIYYA DTVKGRFTIS RDNAKNTLFL
QMTSLRSEDT AMYYCARRGL LLDYWGQGTT LTVS
LIGHT CHAIN scFv SEQUENCE
(SEQ ID NO: XX)
D IVLTQSPASL AVSLGQRATI SCRASKSVST SSYSYMHWYQ
QKPGQPPKLL IKYASYLESG VPARFSGSGS GTDFTLNIHP
VEEEDAATYY CQHSREFPWT FGGGTKLEIK RADAAPGGGG
SGGGGSGGGGS

Molecular modeling of 3E10 (Pymol) revealed a putative Antigen binding pocket (NAB1) (FIGS. 8A-8B). Mutation of aspartic acid at residue 31 of CDR1 to asparagine increased the cationic charge of this residue and enhanced antigen binding and delivery in vivo (3E10-D31N).

Mutation of aspartic acid at residue 31 of CDR1 to arginine (3E10-D31R), further expanded the cationic charge while mutation to lysine (3E10-D31K) changed charge orientation (FIG. 8A).

NAB1 amino acids predicted from molecular modeling have been underlined in the heavy and light chain sequences above. FIG. 8B is an illustration showing molecular modeling of 3E10-scFv (Pymol) with NAB1 amino acid residues illustrated with punctate dots.

### Example 3—3E10 (D31N) Protects mRNA Against RNA Degradation

It was next investigated whether complexing mRNA with 3E10 (D31N) would protect the mRNA from degradation. Briefly, complexes of 3E10 (D31N) and mRNA encoding green fluorescent protein, a luciferase, having the sequence GFP_mRNA shown below as (SEQ ID NO: 138), were formed by mixing 3E10 (D31N) and mRNA at a 20:1 molar ratio. The free mRNA and the 3E10-mRNA complex were then incubated with 1% serum, 10% serum, or 16 μg/mL RNAse A for 10 minutes at 37° C. Gel electrophoresis analysis of the reactions was performed (FIG. 9A). As shown in FIG. 9A, free mRNA was degraded by incubation with each of 1% serum, 10% serum, and RNAse A. However, no apparent RNA degradation was observed when the complexed mRNA was incubated with any of 1% serum, 10% serum, or RNAse A, suggesting that 3E10 (D31N) protects mRNA from degradation.

GFP_mRNA-
(SEQ ID NO: 138)
AUGGUGAGCAAGGGCGAGGAGCUGUUCACCGGGGUGGUGCCCAUCCUGG
UCGAGCUGGACGGCGACGUAAACGGCCACAAGUUCAGCGUGUCCGGCGA
GGGCGAGGGCGAUGCCACCUACGGCAAGCUGACCCUGAAGUUCAUCUGC
ACCACCGGCAAGCUGCCCGUGCCCUGGCCCACCCUCGUGACCACCCUGA
CCUACGGCGUGCAGUGCUUCAGCCGCUACCCCGACCACAUGAAGCAGCA
CGACUUCUUCAAGUCCGCCAUGCCCGAAGGCUACGUCCAGGAGCGCACC
AUCUUCUUCAAGGACGACGGCAACUACAAGACCCGCGCCGAGGUGAAGU
UCGAGGGCGACACCCUGGUGAACCGCAUCGAGCUGAAGGGCAUCGACUU
CAAGGAGGACGGCAACAUCCUGGGGCACAAGCUGGAGUACAACUACAAC
AGCCACAACGUCUAUAUCAUGGCCGACAAGCAGAAGAACGGCAUCAAGG
UGAACUUCAAGAUCCGCCACAACAUCGAGGACGGCAGCGUGCAGCUCGC
CGACCACUACCAGCAGAACACCCCCAUCGGCGACGGCCCCGUGCUGCUG
CCCGACAACCACUACCUGAGCACCCAGUCCGCCCUGAGCAAAGACCCCA
ACGAGAAGCGCGAUCACAUGGUCCUGCUGGAGUUCGUGACCGCCGCCGG
GAUCACUCUCGGCAUGGACGAGCUGUACAAGUAA.

Next, it was investigated whether mRNA complexed at lower molar ratios were also protected against RNA degradation. Briefly, complexes of 3E10 (D3IN) and mRNA encoding green fluorescent protein (GFP_mRNA; SEQ ID NO:138) were formed by mixing 3E10 (D31N) and mRNA at a 2:1 molar ratio. The free mRNA and the 3E10-mRNA complex were then incubated with RNAse A under the conditions described above. Gel electrophoresis analysis of the reactions was performed (FIG. 9B). As shown in FIG. 9B, free mRNA was completely degraded by incubation with RNAse A. However, complexing the mRNA with 3E10 (D31N) at a 2:1 molar ratio resulted in some protection of the mRNA against degradation, as indicated by the presence of an RNA signal in the well, indicating the presence of intact 3E10 (D31N)-mRNA complex. The protection provided at a 2:1 molar ratio appears to be less than the protection afforded the mRNA when complexed at a 20:1 molar ratio.

### Example 4—Distribution of IV Injected 3E10 In Vivo

Dose-dependent biodistribution of 3E10-D31N to tissues was investigated. Mice were injected intravenously with 100 μg or 200 μg of 3E10-D31N labeled with VivoTag680 (Perkin Elmer). Twenty-four hours after injection, tissues were harvested and imaged by IVIS (Perkin Elmer). Specifically, as shown in FIG. 10, a roughly two-fold increase in 3E10 occurred in several tissues when dosing of the composition doubled. Furthermore, 3E10 accumulated in liver tissue at 1 to 2 orders of magnitude more than in other tissues.

### Example 5—3E10-D3IN is Internalized and Associates with gDNA In Vivo

Internalization and cellular location experiments for 3E10-D31N were investigated. Isotype control, 3E10-WT (GMAB^{WT}), and 3E10-D31N (GMAB^{D31N}) antibodies were labeled with ⁸⁹Zr and administered to cells in vivo. After an amount of time, cellular components (cytosol, membrane, nuclear protein, and gDNA) were fractionated and assayed for ⁸⁹Zr signal (Counts per Minute, CPM). As shown in FIG. 11, the majority of the internalized GMAB^{WT} and GMAB^{D31N} antibodies localized to the nucleus and were found associated with both the nuclear protein fraction of the nucleus and the gDNA fraction. However, a larger portion of GMAB^{D31N} associated with the gDNA fraction than did GMAB^{WT}, suggesting that GMAB^{D31N} localizes more readily to chromatin than does GMAB^{WT}.

### Example 6—3E10 (D31N) Protects Dystrophin mRNA Against RNA Degradation

It was investigated whether 3E10-D31N would protect mRNA encoding dystrophin from enzymatic degradation when complexed, and whether larger stochiometric amounts of 3E10-D31N were necessary. Briefly, complexes of 3E10-D31N and a 14 kb mRNA encoding full-length human dystrophin, were formed by mixing 3E10-D31N and mRNA at 1:1, 2:1, 5:1, 10:1, 20:1, and 100:1 molar ratios (3E10: mRNA). The free mRNA and the 3E10-mRNA complexes were then incubated with 6 μg/mL RNAse A for 10 minutes at 37° C. with the addition proteinase K to facilitate protein degradation. FIG. 12 shows agarose gel electrophoresis analysis of the protection assays. As shown in FIG. 12, free dystrophin mRNA, as well as dystrophin mRNA complexed at 1:1, 2:1, 5:1, and 10:1 molar ratios (3E10: mRNA) was completely degraded by incubation with RNAse A. However, as shown in FIG. 12, complexing the dystrophin mRNA with 3E10 at a molar ratio of 20:1 and 100:1 afforded increasing protection of the mRNA from degradation by RNAse A, as indicated by bands migrating at a similar distance as undegraded mRNA on the gel. These results, coupled with those of Example 3, suggest that 3E10 protects polynucleotides in a size-dependent manner.

### Example 7-3E10 (D31N) can Efficiently Deliver Labeled Oligos to Skeletal Muscle in MDX Mice

It was investigated whether 3E10-D31N can efficiently deliver labeled oligos to skeletal muscle in MDX mice. Briefly, a mouse model of muscular dystrophy (MDX) was used to study the biodistribution of a fluorescent oligo. MDX mice were treated with vehicle CTL, a labeled oligo only, or a labeled oligo complexed with 3E10-D3IN. The MDX mice were given a 2-dose or 3-dose treatment regimen (FIGS. 20A and 20B). Twenty four hours after treatment, muscle tissue was harvested and fluorescence was analyzed using an in vivo imaging system measuring Total Radiant Efficiency [p/s][μW/cm²]. These results, as shown in FIG. 20A (2-dose regimen) and FIG. 20B (3-dose regimen), suggest that repeat dosing improve distribution of labeled oligo complexed with 3E10-D31N. This is further illustrated in FIG. 20C, which shows high levels of labeled oligo density in hind leg mouse muscle tissue.

### Example 8—Repeat Dosing of 3E10 (D31N) (V66) Contributed to Increased Antibody Accumulation in Tumors without Commensurate Fluorescence Signal in the Liver

It was investigated whether repeat dosing of a 3E10-D31N “V66” antibody, where the 3E10-VH comprises an amino acid sequence of 3E10-VH-h6 (SEQ ID NO:104) and the 3E10-VL comprises an amino acid sequence of 3E10-VL-h6 (SEQ ID NO:125) in tumor bearing mice will result in increased accumulation of antibody in tumors. Briefly, mice bearing HCT116 tumors were treated 1 or 3 times with PBS (control) or labeled 3E10-D3IN (V66) antibody. Twenty-four hours after the last treated, tumor and liver tissue were harvested and fluorescence was analyzed using an in vivo imaging system measuring Total Radiant Efficiency [p/s][μW/cm²]. The results in FIG. 21A and FIG. 21B, show that repeat dosing increases distribution of labeled 3E10-D31N (V66) in HCT116 tumors. These results, however, are not commensurate with an increased fluorescence signal in the liver. This result is due to IgG clearance in the liver.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

1. A method for treating a hepatic disease in a subject in need thereof, the method comprising:
parenterally administering a therapeutically effective amount of a composition comprising a complex formed between (i) a 3E10 antibody or antigen binding fragment thereof, and (ii) a therapeutic mRNA polynucleotide encoding a hepatic polypeptide.

2. The method of claim 1, wherein the therapeutic mRNA polynucleotide encodes a hepatic polypeptide for which the subject has a loss-of-function mutation.

3. The method of claim 1 or 2, wherein the genetic disease is selected from those diseases listed in Table 2 and the hepatic polypeptide corresponds to the mutant gene for the selected disease.

4. A method for treating a cardiac muscle disease in a subject in need thereof, the method comprising:
parenterally administering a therapeutically effective amount of a composition comprising a complex formed between (i) a 3E10 antibody or antigen binding fragment thereof, and (ii) a therapeutic mRNA polynucleotide encoding a cardiac muscle polypeptide.

5. The method of claim 4, wherein the therapeutic mRNA polynucleotide encodes a cardiac muscle polypeptide for which the subject has a loss-of-function mutation.

6. The method of claim 4 or 5, wherein the genetic disease is selected from those diseases listed in Table 3 and the cardiac muscle polypeptide corresponds to the mutant gene for the selected disease.

7. A method for treating a smooth muscle disease in a subject in need thereof, the method comprising:
parenterally administering a therapeutically effective amount of a composition comprising a complex formed between (i) a 3E10 antibody or antigen binding fragment thereof, and (ii) a therapeutic mRNA polynucleotide encoding a smooth muscle polypeptide.

8. The method of claim 7, wherein the therapeutic mRNA polynucleotide encodes a smooth muscle polypeptide for which the subject has a loss-of-function mutation.

9. The method of claim 7 or 8, wherein the genetic disease is selected from those diseases listed in Table 4 and the smooth muscle polypeptide corresponds to the mutant gene for the selected disease.

10. A method for treating a pulmonary disease in a subject in need thereof, the method comprising:
parenterally administering a therapeutically effective amount of a composition comprising a complex formed between (i) a 3E10 antibody or antigen binding fragment thereof, and (ii) a therapeutic mRNA polynucleotide encoding a pulmonary polypeptide.

11. The method of claim 10, wherein the therapeutic mRNA polynucleotide encodes a pulmonary polypeptide for which the subject has a loss-of-function mutation.

12. The method of claim 10 or 11, wherein the genetic disease is selected from those diseases listed in Table 5 and the pulmonary polypeptide corresponds to the mutant gene for the selected disease.

13. A method for treating a CNS disease in a subject in need thereof, the method comprising:
parenterally administering a therapeutically effective amount of a composition comprising a complex formed between (i) a 3E10 antibody or antigen binding fragment thereof, and (ii) a therapeutic mRNA polynucleotide encoding a CNS polypeptide.

14. The method of claim 13, wherein the therapeutic mRNA polynucleotide encodes a CNS polypeptide for which the subject has a loss-of-function mutation.

15. The method of claim 13 or 14, wherein the genetic disease is selected from those diseases listed in Table 6 and the CNS polypeptide corresponds to the mutant gene for the selected disease.

16. The method according to any one of claims 1-15, wherein the 3E10 antibody or antigen binding fragment thereof comprises:
(a) a light chain variable region (VL) complementarity determining region (CDR) 1 comprising an amino acid sequence having no more than two amino acid substitutions relative to 3E10-VL-CDR1 (SEQ ID NO:9),
(b) a VL CDR2 comprising an amino acid sequence having no more than two amino acid substitutions relative to 3E10-VL-CDR2 (SEQ ID NO:10),
(c) a VL CDR3 comprising an amino acid sequence having no more than two amino acid substitutions relative to 3E10-VL-CDR3 (SEQ ID NO:11),
(d) a heavy chain variable region (VH) CDR1 comprising an amino acid sequence having no more than two amino acid substitutions relative to 3E10-VH-CDR1a (SEQ ID NO:16),
(e) a VH CDR2 comprising an amino acid sequence having no more than two amino acid substitutions relative to 3E10-VH-CDR2 (SEQ ID NO:4), and
(f) a VH CDR3 comprising an amino acid sequence having no more than two amino acid substitutions relative to 3E10-VH-CDR3 (SEQ ID NO:5).

17. The method of claim 16, wherein the VL CDR1 has an amino acid sequence of 3E10-VL-CDR1m (SEQ ID NO:61).

18. The method of claim 16, wherein the VL CDR1 has the amino acid sequence of 3E10-VL-CDR1 (SEQ ID NO:9).

19. The method of any one of claims 16-18, wherein the VL CDR2 has an amino acid sequence of 3E10-VL-CDR2m (SEQ ID NO:62).

20. The method of any one of claims 16-18, wherein the VL CDR2 has the amino acid sequence of 3E10-VL-CDR2 (SEQ ID NO:10).

21. The method of any one of claims 16-20, wherein the VL CDR3 has an amino acid sequence of 3E10-VL-CDR3m (SEQ ID NO:63).

22. The method of any one of claims 16-20, wherein the VL CDR3 has the amino acid sequence of 3E10-VL-CDR3 (SEQ ID NO:11).

23. The method of any one of claims 16-22, wherein the VH CDR1 has an amino acid sequence of 3E10-VH-CDR1m (SEQ ID NO:58).

24. The method of any one of claims 16-22, wherein the VH CDR1 has the amino acid sequence of 3E10-VH-CDR1a (SEQ ID NO:16).

25. The method of any one of claims 16-24, wherein the VH CDR2 has an amino acid sequence selected of 3E10-VH-CDR2m (SEQ ID NO:59).

26. The method of any one of claims 16-24, wherein the VH CDR2 has the amino acid sequence of 3E10-VH-CDR2 (SEQ ID NO:4).

27. The method of any one of claims 16-26, wherein the VH CDR3 has an amino acid sequence of 3E10-VH-CDR3m (SEQ ID NO:60).

28. The method of any one of claims 16-26, wherein the VH CDR3 has the amino acid sequence of 3E10-VH-CDR3 (SEQ ID NO:5).

29. The method according to any one of claims 1-15, wherein the 3E10 antibody or antigen binding fragment thereof comprises:
(a) a light chain variable region (VL) complementarity determining region (CDR) 1 comprising the amino acid sequence of 3E10-VL-CDR1 (SEQ ID NO:9),
(b) a VL CDR2 comprising the amino acid sequence of 3E10-VL-CDR2 (SEQ ID NO: 10),
(c) a VL CDR3 comprising the amino acid sequence of 3E10-VL-CDR3 (SEQ ID NO: 11),
(d) a heavy chain variable region (VH) CDR1 comprising the amino acid sequence of 3E10-VH-CDR1a (SEQ ID NO:16),
(e) a VH CDR2 comprising the amino acid sequence of 3E10-VH-CDR2 (SEQ ID NO: 4), and
(f) a VH CDR3 comprising the amino acid sequence of 3E10-VH-CDR3 (SEQ ID NO:5).

30. The method of any one of claims 1-29, wherein the 3E10 antibody or antigen binding fragment thereof is a humanized 3E10 antibody or antigen-binding fragment thereof.

31. The method of claim 30, wherein the humanized 3E10 antibody or antigen-binding fragment thereof comprises a light chain variable domain (3E10-VL) and a heavy chain variable domain (3E10-VH), wherein:
the 3E10-VL comprises an amino acid sequence that is at least 97% identical to an amino acid sequence selected from the group consisting of 3E10-VL-h1 (SEQ ID NO:120), 3E10-VL-h2 (SEQ ID NO:121), 3E10-VL-h3 (SEQ ID NO:122), 3E10-VL-h4 (SEQ ID NO:123), 3E10-VL-h5 (SEQ ID NO:124), and 3E10-VL-h6 (SEQ ID NO:125), and
the 3E10-VH comprises an amino acid sequence that is at least 95% identical to an amino acid sequence selected from the group consisting of 3E10-VH-h1 (SEQ ID NO:99), 3E10-VH-h2 (SEQ ID NO:100), 3E10-VH-h3 (SEQ ID NO:101), 3E10-VH-h4 (SEQ ID NO:102), 3E10-VH-h5 (SEQ ID NO:103), 3E10-VH-h6 (SEQ ID NO:104), and 3E10-VH-h7 (SEQ ID NO:105).

32. The method of claim 31, wherein the 3E10-VH comprises an amino acid sequence that is at least 95%, 96%, 97%, 98%, 99%, or 100% identical to 3E10-VH-h6 (SEQ ID NO:104) and the 3E10-VL comprises an amino acid sequence that is at least 97%, 98%, 99%, or 100% identical to 3E10-VL-h6 (SEQ ID NO:125).

33. The method of claim 31, wherein the 3E10-VH comprises an amino acid sequence of 3E10-VH-h6 (SEQ ID NO:104) and the 3E10-VL comprises an amino acid sequence of 3E10-VL-h6 (SEQ ID NO:125).

34. The method of claim 30, wherein the humanized 3E10 antibody or antigen-binding fragment thereof comprises a light chain (3E10-LC) and a heavy chain (3E10-HC), wherein:
the 3E10-LC comprises an amino acid sequence that is at least 97% identical to an amino acid sequence selected from the group consisting of 3E10-LC-h1m (SEQ ID NO:126), 3E10-LC-h2m (SEQ ID NO:127), 3E10-LC-h3m (SEQ ID NO:128), 3E10-LC-h4m (SEQ ID NO:129), 3E10-LC-h5m (SEQ ID NO:130), and 3E10-LC-h6m (SEQ ID NO:131), and
the 3E10-HC comprises an amino acid sequence that is at least 95% identical to an amino acid sequence selected from the group consisting of 3E10-HC-h1m (SEQ ID NO:106), 3E10-HC-h2m (SEQ ID NO: 107), 3E10-HC-h3m (SEQ ID NO:108), 3E10-HC-h4m (SEQ ID NO: 109), 3E10-HC-h5m (SEQ ID NO: 110), 3E10-HC-h6m (SEQ ID NO:111), and 3E10-HC-h7m (SEQ ID NO:112).

35. The method of claim 34, wherein the 3E10-HC comprises the amino acid sequence of 3E10-HC-h6m (SEQ ID NO:111) and the 3E10-LC comprises the amino acid sequence of 3E10-LC-h6m (SEQ ID NO:131).

36. The method of claim 34, wherein the 3E10-HC comprises the amino acid sequence of 3E10-HC-h6 (SEQ ID NO:118) and the 3E10-LC comprises the amino acid sequence of 3E10-LC-h6 (SEQ ID NO:137).

37. The method of any one of claims 1-36, wherein the parenteral administration is intramuscular administration, intravenous administration, or subcutaneous administration.

38. The method of any one of claims 1-37, wherein the composition comprises a covalent complex of (i) a 3E10 antibody or antigen binding fragment thereof, and (ii) a therapeutic mRNA polynucleotide.

39. The method of claim 38, wherein the composition comprises a molar ratio of (i) 3E10 antibody or antigen binding fragment thereof to (ii) therapeutic mRNA polynucleotide of at least 1:1.

40. The method of claim 38, wherein the composition comprises a molar ratio of (i) 3E10 antibody or antigen binding fragment thereof to (ii) therapeutic mRNA polynucleotide of at least 1:2.

41. The method of claim 38, wherein the composition comprises a molar ratio of (i) 3E10 antibody or antigen binding fragment thereof to (ii) therapeutic mRNA polynucleotide of at least 1:3.

42. The method of claim 38, wherein the composition comprises a molar ratio of (i) 3E10 antibody or antigen binding fragment thereof to (ii) therapeutic mRNA polynucleotide of at least 1:4.

43. The method of claim 38, wherein the composition comprises a molar ratio of (i) 3E10 antibody or antigen binding fragment thereof to (ii) therapeutic mRNA polynucleotide of at least 1:8.

44. The method of claim 38, wherein the composition comprises a molar ratio of (i) 3E10 antibody or antigen binding fragment thereof to (ii) therapeutic mRNA polynucleotide of at least 1:12.

45. The method of any one of claims 1-44, wherein the composition comprises a non-covalent complex of (i) a 3E10 antibody or antigen binding fragment thereof, and (ii) a therapeutic mRNA polynucleotide.

46. The method of claim 45, wherein the composition comprises a molar ratio of (i) 3E10 antibody or antigen binding fragment thereof to (ii) therapeutic mRNA polynucleotide of at least 2:1.

47. The method of claim 45, wherein the composition comprises a molar ratio of (i) 3E10 antibody or antigen binding fragment thereof to (ii) therapeutic mRNA polynucleotide of at least 5:1, at least 20:1, at least 50:1, or at least 100:1.

48. The method of any one of claims 45-47, wherein the composition comprises a molar ratio of (i) 3E10 antibody or antigen binding fragment thereof to (ii) therapeutic polynucleotide of no more than 200:1, no more than 100:1, or no more than 50:1.

49. The method of claim 45, wherein the composition comprises a molar ratio of (i) 3E10 antibody or antigen binding fragment thereof to (ii) therapeutic polynucleotide of from 2:1 to 200:1 or from 5:1 to 200:1.

50. The method of claim 45, wherein the composition comprises a molar ratio of (i) 3E10 antibody or antigen binding fragment thereof to (ii) therapeutic polynucleotide of from 2:1 to 50:1, wherein the therapeutic polynucleotide is no more than 2000 nucleotides in length.

51. The method of claim 45, wherein the composition comprises a molar ratio of (i) 3E10 antibody or antigen binding fragment thereof to (ii) therapeutic polynucleotide of from 2:1 to 30:1, wherein the therapeutic polynucleotide is no more than 1000 nucleotides in length.

52. The method of claim 45, wherein the composition comprises a molar ratio of (i) 3E10 antibody or antigen binding fragment thereof to (ii) therapeutic polynucleotide of from 20:1 to 200:1, wherein the therapeutic polynucleotide is at least 2000 nucleotides in length.

53. The method of any one of claims 1-52, wherein the antibody or fragment thereof comprises:
a heavy chain comprising, from N- to C-terminal, VH-CH1-hinge-CH2-CH3, and
a light chain comprising, from N- to C-terminal, VL-CL.

54. The method of claim 53, wherein the hinge-CH2-CH3 is an Fc domain selected from the group consisting of the Fc domain from human IgG1, IgG2, IgG3 and IgG4.

55. A pharmaceutical composition comprising a complex formed between (i) a 3E10 antibody or antigen binding fragment thereof, and (ii) a therapeutic mRNA polynucleotide, wherein the pharmaceutical composition comprises a molar ratio of (i) 3E10 antibody or antigen binding fragment thereof to (ii) therapeutic polynucleotide of at least 2:1.

56. The pharmaceutical composition of claim 55, wherein the therapeutic mRNA polynucleotide encodes a hepatic polypeptide.

57. The pharmaceutical composition of claim 56, wherein the hepatic polypeptide is selected from those listed in Table 2.

58. The pharmaceutical composition of claim 55, wherein the therapeutic mRNA polynucleotide encodes a cardiac muscle polypeptide.

59. The pharmaceutical composition of claim 58, wherein the cardiac muscle polypeptide is selected from those listed in Table 3.

60. The pharmaceutical composition of claim 55, wherein the therapeutic mRNA polynucleotide encodes a smooth muscle polypeptide.

61. The pharmaceutical composition of claim 60, wherein the smooth muscle polypeptide is selected from those listed in Table 4.

62. The pharmaceutical composition of claim 55, wherein the therapeutic mRNA polynucleotide encodes a pulmonary polypeptide.

63. The pharmaceutical composition of claim 62, wherein the pulmonary polypeptide is selected from those listed in Table 5.

64. The pharmaceutical composition of claim 55, wherein the therapeutic mRNA polynucleotide encodes a CNS polypeptide.

65. The pharmaceutical composition of claim 64, wherein the CNS polypeptide is selected from those listed in Table 6.

66. The pharmaceutical composition according to any one of claims 55-65, wherein the 3E10 antibody or antigen binding fragment thereof comprises:
(a) a light chain variable region (VL) complementarity determining region (CDR) 1 comprising an amino acid sequence having no more than two amino acid substitutions relative to 3E10-VL-CDR1 (SEQ ID NO:9),
(b) a VL CDR2 comprising an amino acid sequence having no more than two amino acid substitutions relative to 3E10-VL-CDR2 (SEQ ID NO:10),
(c) a VL CDR3 comprising an amino acid sequence having no more than two amino acid substitutions relative to 3E10-VL-CDR3 (SEQ ID NO:11),
(d) a heavy chain variable region (VH) CDR1 comprising an amino acid sequence having no more than two amino acid substitutions relative to 3E10-VH-CDR1a (SEQ ID NO:16),
(e) a VH CDR2 comprising an amino acid sequence having no more than two amino acid substitutions relative to 3E10-VH-CDR2 (SEQ ID NO:4), and
(f) a VH CDR3 comprising an amino acid sequence having no more than two amino acid substitutions relative to 3E10-VH-CDR3 (SEQ ID NO:5).

67. The pharmaceutical composition of claim 66, wherein the VL CDR1 has an amino acid sequence of 3E10-VL-CDR1m (SEQ ID NO:61).

68. The pharmaceutical composition of claim 66, wherein the VL CDR1 has the amino acid sequence of 3E10-VL-CDR1 (SEQ ID NO:9).

69. The pharmaceutical composition of any one of claims 66-68, wherein the VL CDR2 has an amino acid sequence of 3E10-VL-CDR2m (SEQ ID NO:62).

70. The pharmaceutical composition of any one of claims 66-68, wherein the VL CDR2 has the amino acid sequence of 3E10-VL-CDR2 (SEQ ID NO:10).

71. The pharmaceutical composition of any one of claims 66-70, wherein the VL CDR3 has an amino acid sequence of 3E10-VL-CDR3m (SEQ ID NO:63).

72. The pharmaceutical composition of any one of claims 66-70, wherein the VL CDR3 has the amino acid sequence of 3E10-VL-CDR3 (SEQ ID NO:11).

73. The pharmaceutical composition of any one of claims 66-72, wherein the VH CDR1 has an amino acid sequence of 3E10-VH-CDR1m (SEQ ID NO:58).

74. The pharmaceutical composition of any one of claims 66-72, wherein the VH CDR1 has the amino acid sequence of 3E10-VH-CDR1a (SEQ ID NO:16).

75. The pharmaceutical composition of any one of claims 66-74, wherein the VH CDR2 has an amino acid sequence selected of 3E10-VH-CDR2m (SEQ ID NO:59).

76. The pharmaceutical composition of any one of claims 66-74, wherein the VH CDR2 has the amino acid sequence of 3E10-VH-CDR2 (SEQ ID NO:4).

77. The pharmaceutical composition of any one of claims 66-76, wherein the VH CDR3 has an amino acid sequence of 3E10-VH-CDR3m (SEQ ID NO:60).

78. The pharmaceutical composition of any one of claims 66-76, wherein the VH CDR3 has the amino acid sequence of 3E10-VH-CDR3 (SEQ ID NO:5).

79. The pharmaceutical composition according to any one of claims 55-65, wherein the 3E10 antibody or antigen binding fragment thereof comprises:
(a) a light chain variable region (VL) complementarity determining region (CDR) 1 comprising the amino acid sequence of 3E10-VL-CDR1 (SEQ ID NO:9),
(b) a VL CDR2 comprising the amino acid sequence of 3E10-VL-CDR2 (SEQ ID NO: 10),
(c) a VL CDR3 comprising the amino acid sequence of 3E10-VL-CDR3 (SEQ ID NO: 11),
(d) a heavy chain variable region (VH) CDR1 comprising the amino acid sequence of 3E10-VH-CDR1a (SEQ ID NO:16),
(e) a VH CDR2 comprising the amino acid sequence of 3E10-VH-CDR2 (SEQ ID NO: 4), and
(f) a VH CDR3 comprising the amino acid sequence of 3E10-VH-CDR3 (SEQ ID NO:5).

80. The pharmaceutical composition of any one of claims 55-79, wherein the 3E10 antibody or antigen binding fragment thereof is a humanized 3E10 antibody or antigen-binding fragment thereof.

81. The pharmaceutical composition of claim 80, wherein the humanized 3E10 antibody or antigen-binding fragment thereof comprises a light chain variable domain (3E10-VL) and a heavy chain variable domain (3E10-VH), wherein:
the 3E10-VL comprises an amino acid sequence that is at least 97% identical to an amino acid sequence selected from the group consisting of 3E10-VL-h1 (SEQ ID NO:120), 3E10-VL-h2 (SEQ ID NO:121), 3E10-VL-h3 (SEQ ID NO:122), 3E10-VL-h4 (SEQ ID NO:123), 3E10-VL-h5 (SEQ ID NO:124), and 3E10-VL-h6 (SEQ ID NO:125), and
the 3E10-VH comprises an amino acid sequence that is at least 95% identical to an amino acid sequence selected from the group consisting of 3E10-VH-h1 (SEQ ID NO:99), 3E10-VH-h2 (SEQ ID NO:100), 3E10-VH-h3 (SEQ ID NO:101), 3E10-VH-h4 (SEQ ID NO:102), 3E10-VH-h5 (SEQ ID NO:103), 3E10-VH-h6 (SEQ ID NO:104), and 3E10-VH-h7 (SEQ ID NO:105).

82. The pharmaceutical composition of claim 81, wherein the 3E10-VH comprises an amino acid sequence that is at least 95%, 96%, 97%, 98%, 99%, or 100% identical to 3E10-VH-h6 (SEQ ID NO:104) and the 3E10-VL comprises an amino acid sequence that is at least 97%, 98%, 99%, or 100% identical to 3E10-VL-h6 (SEQ ID NO:125).

83. The pharmaceutical composition of claim 81, wherein the 3E10-VH comprises an amino acid sequence of 3E10-VH-h6 (SEQ ID NO: 104) and the 3E10-VL comprises an amino acid sequence of 3E10-VL-h6 (SEQ ID NO:125).

84. The pharmaceutical composition of claim 80, wherein the humanized 3E10 antibody or antigen-binding fragment thereof comprises a light chain (3E10-LC) and a heavy chain (3E10-HC), wherein:
the 3E10-LC comprises an amino acid sequence that is at least 97% identical to an amino acid sequence selected from the group consisting of 3E10-LC-h1m (SEQ ID NO:126), 3E10-LC-h2m (SEQ ID NO:127), 3E10-LC-h3m (SEQ ID NO:128), 3E10-LC-h4m (SEQ ID NO:129), 3E10-LC-h5m (SEQ ID NO:130), and 3E10-LC-h6m (SEQ ID NO:131), and
the 3E10-HC comprises an amino acid sequence that is at least 95% identical to an amino acid sequence selected from the group consisting of 3E10-HC-h1m (SEQ ID NO:106), 3E10-HC-h2m (SEQ ID NO:107), 3E10-HC-h3m (SEQ ID NO:108), 3E10-HC-h4m (SEQ ID NO: 109), 3E10-HC-h5m (SEQ ID NO:110), 3E10-HC-h6m (SEQ ID NO:111), and 3E10-HC-h7m (SEQ ID NO:112).

85. The pharmaceutical composition of claim 80, wherein the 3E10-HC comprises the amino acid sequence of 3E10-HC-h6m (SEQ ID NO:111) and the 3E10-LC comprises the amino acid sequence of 3E10-LC-h6m (SEQ ID NO:131).

86. The pharmaceutical composition of claim 80, wherein the 3E10-HC comprises the amino acid sequence of 3E10-HC-h6 (SEQ ID NO:118) and the 3E10-LC comprises the amino acid sequence of 3E10-LC-h6 (SEQ ID NO:137).

87. The pharmaceutical composition of any one of claims 55-86, wherein the parenteral administration is intramuscular administration, intravenous administration, or subcutaneous administration.

88. The pharmaceutical composition of any one of claims 55-87, wherein the composition comprises a covalent complex of (i) a 3E10 antibody or antigen binding fragment thereof, and (ii) a therapeutic mRNA polynucleotide.

89. The pharmaceutical composition of claim 88, wherein the composition comprises a molar ratio of (i) 3E10 antibody or antigen binding fragment thereof to (ii) therapeutic mRNA polynucleotide of at least 1:1.

90. The pharmaceutical composition of claim 88, wherein the composition comprises a molar ratio of (i) 3E10 antibody or antigen binding fragment thereof to (ii) therapeutic mRNA polynucleotide of at least 1:2.

91. The pharmaceutical composition of claim 88, wherein the composition comprises a molar ratio of (i) 3E10 antibody or antigen binding fragment thereof to (ii) therapeutic mRNA polynucleotide of at least 1:3.

92. The pharmaceutical composition of claim 88, wherein the composition comprises a molar ratio of (i) 3E10 antibody or antigen binding fragment thereof to (ii) therapeutic mRNA polynucleotide of at least 1:4.

93. The pharmaceutical composition of claim 88, wherein the composition comprises a molar ratio of (i) 3E10 antibody or antigen binding fragment thereof to (ii) therapeutic mRNA polynucleotide of at least 1:8.

94. The pharmaceutical composition of claim 88, wherein the composition comprises a molar ratio of (i) 3E10 antibody or antigen binding fragment thereof to (ii) therapeutic mRNA polynucleotide of at least 1:12.

95. The pharmaceutical composition of any one of claims 55-87, wherein the composition comprises a non-covalent complex of (i) a 3E10 antibody or antigen binding fragment thereof, and (ii) a therapeutic mRNA polynucleotide.

96. The pharmaceutical composition of claim 95, wherein the composition comprises a molar ratio of (i) 3E10 antibody or antigen binding fragment thereof to (ii) therapeutic mRNA polynucleotide of at least 2:1.

97. The pharmaceutical composition of claim 95, wherein the composition comprises a molar ratio of (i) 3E10 antibody or antigen binding fragment thereof to (ii) therapeutic mRNA polynucleotide of at least 5:1, at least 20:1, at least 50:1, or at least 100:1.

98. The pharmaceutical composition of any one of claims 95-97, wherein the composition comprises a molar ratio of (i) 3E10 antibody or antigen binding fragment thereof to (ii) therapeutic polynucleotide of no more than 200:1, no more than 100:1, or no more than 50:1.

99. The pharmaceutical composition of claim 95, wherein the composition comprises a molar ratio of (i) 3E10 antibody or antigen binding fragment thereof to (ii) therapeutic polynucleotide of from 2:1 to 200:1 or from 5:1 to 200:1.

100. The pharmaceutical composition of claim 95, wherein the composition comprises a molar ratio of (i) 3E10 antibody or antigen binding fragment thereof to (ii) therapeutic polynucleotide of from 2:1 to 50:1, wherein the therapeutic polynucleotide is no more than 2000 nucleotides in length.

101. The pharmaceutical composition of claim 95, wherein the composition comprises a molar ratio of (i) 3E10 antibody or antigen binding fragment thereof to (ii) therapeutic polynucleotide of from 2:1 to 30:1, wherein the therapeutic polynucleotide is no more than 1000 nucleotides in length.

102. The pharmaceutical composition of claim 95, wherein the composition comprises a molar ratio of (i) 3E10 antibody or antigen binding fragment thereof to (ii) therapeutic polynucleotide of from 20:1 to 200:1, wherein the therapeutic polynucleotide is at least 2000 nucleotides in length.

103. The pharmaceutical composition of any one of claims 55-102, wherein the antibody or fragment thereof comprises:
a heavy chain comprising, from N- to C-terminal, VH-CH1-hinge-CH2-CH3, and
a light chain comprising, from N- to C-terminal, VL-CL.

104. The pharmaceutical composition of claim 103, wherein the hinge-CH2-CH3 is an Fc domain selected from the group consisting of the Fc domain from human IgG1, IgG2, IgG3 and IgG4.
